(19) United States
(12) Patent Application Publication
(10) Pub. No.: US 2020/0277405 A1
(43) Pub. Date: Sep. 3, 2020

(54) COMPOSITION FOR ANTIBODY-DRUG CONJUGATE DIRECTED AGAINST TUMOR-CELL ASSOCIATED POLYSIALIC ACID

(71) Applicant: Cornell University, Ithaca, NY (US)

(72) Inventors: Matthew P. DELISA, Ithaca, NY (US); Emily C. COX, Hector, NY (US); Christopher A. ALABI, Ithaca, NY (US); Dana N. THORNLOW, Sayville, NY (US)

(21) Appl. No.: 16/791,509

(22) Filed: Feb. 14, 2020

Related U.S. Application Data
(60) Provisional application No. 62/805,752, filed on Feb. 14, 2019.

(51) Int. Cl. C07K 16/44; A61K 47/68

(52) U.S. Cl. CPC C07K 16/44; A61K 45/06; A61K 47/6803

(57) ABSTRACT

The present application discloses an immunoconjugate therapeutic comprising a polysialic acid targeting portion and an anti-cancer therapeutic coupled to the polysialic acid targeting portion. The present application also discloses methods of treating subjects with cancer and methods of targeting intracellular delivery of an anti-cancer therapeutic to a target cell population with the immunoconjugate therapeutic.

This application claims the benefit of U.S. Provisional Patent Application Ser. No. 62/805,752, filed Feb. 14, 2019, which is hereby incorporated by reference in its entirety.

This invention was made with government support under CBET-1605242 awarded by the National Science Foundation and GRANT11631647 awarded by the Defense Threat Reduction Agency. The government has certain rights in the invention.

### FIELD

The present application relates to immunoconjugate therapeutics, methods of treating subjects with cancer, and methods of targeting intracellular delivery of an anti-cancer therapeutic.

### BACKGROUND

The specific targeting of glycans that differentially occur in malignant cells has emerged as an attractive anti-cancer strategy. One such target is the oncodevelopmental antigen polysialic acid (polySia), a polymer of α2,8-linked sialic acid residues that is largely absent during postnatal development but is re-expressed during progression of several malignant human tumors including small cell and non-small cell lung carcinomas, glioma, neuroblastoma, and pancreatic carcinoma. In these cancers, the expression of polySia is correlated with tumor progression and poor prognosis, and also appears to modulate cancer cell adhesion, invasiveness, and metastasis. To evaluate the potential of PolySia as a target for anti-cancer therapy, a chimeric human polySia-specific monoclonal antibody (mAb) was developed that retained low nanomolar (nM) target affinity and exhibited exquisite selectivity for polySia structures. Using flow cytometry and confocal microscopy, it was confirmed that the engineered chimeric mAb recognized several polySia-positive tumor cell lines in vitro and induced rapid endocytosis of polySia antigens. To determine if this internalization could be exploited for delivery of conjugated cytotoxic drugs, an antibody-drug conjugate (ADC) was generated by covalently linking the chimeric human mAb to the tubulin-binding maytansinoid DM1 using a bioorthogonal chemical reaction scheme. The resulting polySia-directed ADC demonstrated potent target-dependent cytotoxicity against polySia-positive tumor cells in vitro. Collectively, these results establish polySia as a valid cell-surface, cancer-specific target for glycan-directed ADC and contribute to a growing body of evidence that the tumor glycocalyx is a promising target for synthetic immunotherapies.

Glycosylation is the site-specific attachment of sugar assemblies known as glycans to a functional group of another molecule, most commonly proteins or lipids, resulting in the formation of a glycoconjugate. It is a tightly controlled cell- and microenvironment-specific mechanism that involves the coordinated expression and activity of numerous enzymes such as glycosyltransferases and glycosidases. Cellular glycosylation and its products are fundamental to a diverse range of biological processes involved in cancer progression including cell growth and proliferation, cell signaling and communication, cell-cell and cell-extracellular matrix (ECM) interactions, and immune recognition/response (Fuster et al., “The Sweet and Sour of Cancer: Glycans as Novel Therapeutic Targets,” Nature Reviews Cancer 5:526-42 (2005); Marth et al., “Mammalian Glycosylation in Immunity,” Nat. Rev. Immunol. 8:874-87 (2008); Stowell et al., “Protein Glycosylation in Cancer,” Annu. Rev. Pathol. 10:473-510 (2015); and Ohtsubo et al., “Glycosylation in Cellular Mechanisms of Health and Disease,” Cell 126:855-67 (2006)). Thus, it is not surprising that nearly all types of human cancers exhibit changes in glycosylation, a phenomenon that was first reported more than six decades ago (Hakomori et al., “Glycolipids of Hamster Fibroblasts and Derived Malignant-Transformed Cell Lines,” Proc. Natl. Acad. Sci. U.S.A. 59:254-61 (1968) and Ladenson et al., “Incidence of the Blood Groups and the Secretor Factor in Patients with Pernicious Anemia and Stomach Carcinoma,” Am. J. Med. Sci. 217:194-7 (1949)). The glycosylation changes associated with oncogenic transformation typically involve either incomplete synthesis or neo-synthesis processes, both of which may arise from under- or overexpression of glycosyltransferases and glycosidases leading to the exposure of aberrant cell-surface glycans. The most common cancer-associated structural changes include N- and O-glycan branching, O-glycan truncation, increased sialylation, and increased “core” fucosylation, with these motifs occurring on all classes of glycoconjugates including glycoproteins, glycosphingolipids, and proteoglycans (Pinho et al., “Glycosylation in Cancer: Mechanisms and Clinical Implications,” Nature Reviews Cancer 15:540-55 (2015) and Dube et al., “Glycans in Cancer and Inflammation—Potential for Therapeutics and Diagnostics,” Nat. Rev. Drug Discov. 4:477-88 (2005)).

Many of these abnormal glycan epitopes are differentially expressed on malignant cells, thereby providing novel diagnostic and even therapeutic targets that are motivating the development of affinity reagents that recognize these distinct features. However, whereas a rich and diverse collection of antibodies and antibody-derived molecules have been developed for protein antigens, reliable binders that specifically recognize carbohydrates are much less common. Indeed, the paucity of glycan-specific binding reagents was noted by the National Academy of Sciences as a key barrier for advancing glycobiology (National Research Council (US) Committee on Assessing the Importance and Impact of Glycomics and Glycosciences, Transforming Glycoscience: A Roadmap for the Future, Washington (DC), National Academies Press (US) (2012)). This shortage was also highlighted in the recently assembled Database for Anti-Glycan Reagents (DAGR), which indicates that while there are ˜100 entries for antibodies against N- and O-linked carbohydrates, collectively these target an extremely small set of unique epitopes (Sterner et al., “Perspectives on Anti-Glycan Antibodies Gleaned from Development of a Community Resource Database,” ACS Chem. Biol. 11:1773-83 (2016)). Specifically, 55 of the 77 total antibodies to O-linked glycans target Tn, sialyl Tn, or TF antigens while 15 of the 25 total antibodies to N-linked glycans are derived from HIV patients. There is clearly a technological deficit when one considers that glycoproteins and glycolipids are estimated to contain approximately 3,000 glycan determinants (Cummings R D., “The Repertoire of Glycan Determinants in the Human Glycome,” Mol. Biosyst. 5:1087-104 (2009)).

Even when anti-glycan antibodies are available, information about their specificity is often limited and, in a surprising number of cases, antibodies reported to be specific for a designated antigen were found to cross-react with other glycans (Manimala et al., “High-Throughput Carbohydrate Microarray Profiling of 27 Antibodies Demonstrates Widespread Specificity Problems,” Glycobiology 17:17C-23C (2007)). Moreover, for many of the glycans that differentially occur in malignant cells, it remains to be determined whether they are druggable using “synthetic” immunotherapies (Majzner et al., “Harnessing the Immunotherapy Revolution for the Treatment of Childhood Cancers,” Cancer Cell 31:476-85 (2017)) such as monoclonal antibodies (mAbs), antibody-drug conjugates (ADCs), bispecific antibodies (BsAbs), and chimeric antigen receptors (CARs), which all have the potential to initiate new immune or immune-like responses directed toward their tumor-expressed targets. One notable example along these lines is the synthetic immunotherapy dinutuximab, a first-in-class monoclonal antibody (mAb) that recognizes the disialoganglioside GD2 found on the surface of neuroblastic tumor cells (Barker et al., “Effect of a Chimeric Anti-Ganglioside GD2 Antibody on Cell-Mediated Lysis of Human Neuroblastoma Cells,” Cancer Res. 51:144-9 (1991)) and is administered as part of a multi-agent, multimodality therapy to pediatric patients with high-risk neuroblastoma (Yu et al., “Anti-GD2 Antibody with GM-CSF, Interleukin-2, and Isotretinoin for Neuroblastoma,” N. Engl. J. Med. 363:1324-34 (2010)). There is similar potential to develop other glycan-directed antibodies and antibody derivatives; however, this will require overcoming a number of key obstacles related to (i) the current lack of antibodies against structurally diverse glycan antigens beyond the small subset discussed above and (ii) the incomplete knowledgebase surrounding known antibodies in terms of their performance characteristics such as target specificity and therapeutic function (e.g., cytotoxicity).

Here, this latter gap is addressed by systematically characterizing the well-known mouse-derived mAb 735 (mo735) (Frosch et al., “NZB Mouse System for Production of Monoclonal Antibodies to Weak Bacterial Antigens: Isolation of an IgG Antibody to the Polysaccharide Capsules of Escherichia coli K1 and Group B Meningococci,” Proc. Natl. Acad. Sci. U.S.A. 82:1194-8 (1985)) and a newly created chimerized human derivative (ch735), both of which target the oncodevelopmental carbohydrate antigen polysialic acid (polySia). PolySia is a unique glycan homopolymer of α2,8-linked N-acetyl neuraminic acid (NeuNAc) that occurs as a terminating structure on the N-linked glycan associated with the neural cell adhesion molecule (NCAM) and also as a capsular polysaccharide (CPS) on the surface of bacterial pathogens causing meningitis (Colley et al., “Polysialic Acid: Biosynthesis, Novel Functions and Applications,” Critical Reviews in Biochemistry and Molecular Biology 49:498-532 (2014)). In vertebrates, the expression of polySia is abundant during early stages of development of the brain, heart, kidney, liver, pancreas, respiratory and digestive tracts, but becomes significantly reduced in adults with expression largely restricted to certain regions of the brain (Colley et al., “Polysialic Acid: Biosynthesis, Novel Functions and Applications,” Critical Reviews in Biochemistry and Molecular Biology 49:498-532 (2014) and Galuska et al., “Is Polysialylated NCAM not Only a Regulator During Brain Development but also During the Formation of Other Organs?,” Biology (Basel) 6(2):27 (2017)). Importantly, it is aberrantly re-expressed in many cancers, appearing as part of the tumor glycocalyx in small cell lung cancer (SCLC) (Kibbelaar et al., “Expression of the Embryonal Neural Cell Adhesion Molecule N-CAM in Lung Carcinoma: Diagnostic Usefulness of Monoclonal Antibody 735 for the Distinction Between Small Cell Lung Cancer and Non-Small Cell Lung Cancer,” J. Pathol. 159:23-8 (1989)), non-small cell lung cancer (NSCLC) (Tanaka et al., “Expression of Polysialic Acid and STX, a Human Polysialyltransferase, is Correlated with Tumor Progression in Non-Small Cell Lung Cancer,” Cancer Res. 60:3072-80 (2000)), pancreatic cancer (Kameda et al., “Expression of Highly Polysialylated Neural Cell Adhesion Molecule in Pancreatic Cancer Neural Invasive Lesion,” Cancer Lett. 137:201-7 (1999)), Wilm's tumor (Roth et al., “Reexpression of Poly(Sialic Acid) Units of the Neural Cell Adhesion Molecule in Wilms Tumor,” Proc. Natl. Acad. Sci. U.S.A. 85:2999-3003 (1988)), neuroblastoma (Livingston et al., “Extended Polysialic Acid Chains (N Greater than 55) in Glycoproteins from Human Neuroblastoma Cells,” J. Biol. Chem. 263:9443-8 (1988)), and glioma (Suzuki et al., “Polysialic Acid Facilitates Tumor Invasion by Glioma Cells,” Glycobiology 15:887-94 (2005)) among others. PolySia expression, which is catalyzed by two polysialyltransferases, ST8SiaIV (PST) and particularly ST8SiaII (STX) in cancer cells (Tanaka et al., “Expression of Polysialic Acid and STX, a Human Polysialyltransferase, is Correlated with Tumor Progression in Non-Small Cell Lung Cancer,” Cancer Res. 60:3072-80 (2000)), is known to promote cancer cell adhesion, migration and invasion (Suzuki et al., “Polysialic Acid Facilitates Tumor Invasion by Glioma Cells,” Glycobiology 15:887-94 (2005); Daniel et al., “A Nude Mice Model of Human Rhabdomyosarcoma Lung Metastases for Evaluating the Role of Polysialic Acids in the Metastatic Process,” Oncogene 20:997-1004 (2001); Scheidegger et al., “In vitro and in vivo Growth of Clonal Sublines of Human Small Cell Lung Carcinoma is Modulated by Polysialic Acid of the Neural Cell Adhesion Molecule,” Laboratory Investigation; A Journal of Technical Methods and Pathology 70:95-106 (1994); and Hromatka et al., “Polysialic Acid Enhances the Migration and Invasion of Human Cytotrophoblasts,” Glycobiology 23:593-602 (2013)) and is strongly correlated with aggressive and metastatic disease as well as poor prognosis in the clinic (Falconer et al., “Polysialyltransferase: A New Target in Metastatic Cancer,” Curr. Cancer Drug Targets 12:925-39 (2012)). For many of the aforementioned reasons, polySia was ranked as the second highest priority glycan antigen (after GD2) in a National Cancer Institute pilot project (Cheever et al., “The Prioritization of Cancer Antigens: A National Cancer Institute Pilot Project for the Acceleration of Translational Research,” Clin. Cancer Res. 15:5323-37 (2009)).

### SUMMARY

One aspect of the present application is an immunoconjugate therapeutic comprising a polysialic acid targeting portion and an anti-cancer therapeutic coupled to the polysialic acid targeting portion.

Another aspect of the present application is a method of treating subjects with cancer, said method comprising selecting a subject with cancer characterized by polysialic acid (polySia)-positive tumor cells and administering an immunoconjugate therapeutic of the present application to the selected subject.

Another aspect of the present application is a method of targeted intracellular delivery of an anti-cancer therapeutic to a target cell population, said method comprising selecting a population of target cells, wherein the population of target cells is positive for polysialic acid (polySia) and administering an immunoconjugate therapeutic of the present application to the selected target cell population.

Collectively, the results described here validate polySia as a therapeutically tractable target for ADC and pave the way for achieving selective cytotoxic effects against tumors that aberrantly express this unique oncodevelopmental antigen. The choice of polySia as a therapeutic target is supported by the fact that polySia is expressed throughout the fetus and during embryonic development, but in adults polySia expression is highly restricted (Colley et al., “Polysialic Acid: Biosynthesis, Novel Functions and Applications,” Critical Reviews in Biochemistry and Molecular Biology 49:498-532 (2014); Galuska et al., “Is Polysialylated NCAM not Only a Regulator During Brain Development but also During the Formation of Other Organs?,” Biology (Basel) 6(2):27 (2017); and Zhang et al., “Selection of Tumor Antigens as Targets for Immune Attack using Immunohistochemistry: i. Focus on Gangliosides,” International Journal of Cancer 73:42-9 (1997), which are hereby incorporated by reference in their entirety).

Specifically, according to previously published IHC results, the mo735 mAb reacted with only a limited number of cells and tissues including gray matter of brain, bronchial epithelia and pneumocytes, and capillary endothelial cells and ganglion neurons in the colon (Zhang et al., “Selection of Tumor Antigens as Targets for Immune Attack using Immunohistochemistry: i. Focus on Gangliosides,” International Journal of Cancer 73:42-9 (1997), which is hereby incorporated by reference in its entirety). Importantly, polySia is re-expressed in many types of cancer including SCLC (Kibbelaar et al., “Expression of the Embryonal Neural Cell Adhesion Molecule N-CAM in Lung Carcinoma: Diagnostic Usefulness of Monoclonal Antibody 735 for the Distinction Between Small Cell Lung Cancer and Non-Small Cell Lung Cancer,” J. Pathol. 159:23-8 (1989), which is hereby incorporated by reference in its entirety), NSCLC (Tanaka et al., “Expression of Polysialic Acid and STX, a Human Polysialyltransferase, is Correlated with Tumor Progression in Non-Small Cell Lung Cancer,” Cancer Res. 60:3072-80 (2000), which is hereby incorporated by reference in its entirety), pancreatic cancer (Kameda et al., “Expression of Highly Polysialylated Neural Cell Adhesion Molecule in Pancreatic Cancer Neural Invasive Lesion,” Cancer Lett. 137:201-7 (1999), which is hereby incorporated by reference in its entirety), Wilm's tumor (Roth et al., “Reexpression of Poly(Sialic Acid) Units of the Neural Cell Adhesion Molecule in Wilms Tumor,” Proc. Natl. Acad. Sci. U.S.A. 85:2999-3003 (1988), which is hereby incorporated by reference in its entirety), neuroblastoma (Livingston et al., “Extended Polysialic Acid Chains (N Greater than 55) in Glycoproteins from Human Neuroblastoma Cells,” J. Biol. Chem. 263:9443-8 (1988), which is hereby incorporated by reference in its entirety), and glioma (Suzuki et al., “Polysialic Acid Facilitates Tumor Invasion by Glioma Cells,” Glycobiology 15:887-94 (2005), which is hereby incorporated by reference in its entirety), and its increased expression typically correlates with later stages and increased invasive and metastatic potential (Falconer et al., “Polysialyltransferase: A New Target in Metastatic Cancer,” Curr. Cancer Drug Targets 12:925-39 (2012), which is hereby incorporated by reference in its entirety).

While recent reports indicate that polySia is also expressed on certain human immune cells (Drake et al., “Polysialic Acid, A Glycan with Highly Restricted Expression, Is Found on Human and Murine Leukocytes and Modulates Immune Responses,” The Journal of Immunology 181:6850-8 (2008) and Curreli et al., ‘Polysialylated Neuropilin-2 is Expressed on the Surface of Human Dendritic Cells and Modulates Dendritic Cell-T Lymphocyte Interactions,” J. Biol. Chem. 282:30346-56 (2007), which are hereby incorporated by reference in their entirety), this expression appears to be quite heterogenous and is progressively down-regulated in wild-type monocytes and monocyte-derived cells during migration from bone marrow (BM) through peripheral blood (PB) to pulmonary and peritoneal sites of inflammation, with levels in PB and inflammation sites reported to be extremely low or absent relative to the levels detected in BM (Stamatos et al., “Changes in Polysialic Acid Expression on Myeloid Cells During Differentiation and Recruitment to Sites of Inflammation: Role in Phagocytosis,” Glycobiology 24:864-79 (2014), which is hereby incorporated by reference in its entirety). Regarding polySia's occurrence on healthy cells, it should be pointed out that Schneerson and colleagues conducted a thorough review of published data looking for evidence that anti-polySia IgG antibodies caused immunopathology in humans. From their study, they found no evidence of increased autoimmunity and urged that the use of anti-polySia immunotherapies be considered (Stein et al., “Are Antibodies to the Capsular Polysaccharide of Neisseria Meningitidis Group B and Escherichia Coli K1 Associated with Immunopathology?,” Vaccine 24:221-8 (2006), which is hereby incorporated by reference in its entirety). Further studies on the therapeutic targeting of polySia and the safety of such an approach are highly warranted, especially in light of the results presented here.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole. In addition, preferences and options for a given aspect, feature, embodiment, or parameter of the invention should, unless the context indicates otherwise, be regarded as having been disclosed in combination with any and all preferences and options for all other aspects, features, embodiments, and parameters of the present application.

### BRIEF DESCRIPTION OF THE DRAWINGS

FIGS. 1A-1C depict expression and purification of ch735. FIG. 1A shows antibody expression yields determined following purification from a HEK 293-F cell culture that was transfected with pVITRO1-735-IgG1/κ and subsequently selected with hygromycin B for two weeks to generate a stable line expressing ch735. Protein quantification was performed by measuring absorbance at 280 nm (Abs₂₈₀). FIG. 1B shows recombinant purified ch735 analyzed by Western blotting and Coomassie blue staining under non-reducing and reducing conditions as indicated. Blots were probed with anti-human IgG Fc antibody to detect fully assembled antibody and the reduced heavy chain and anti-human kappa light chain antibody to detect the light chain. Molecular weight (MW) markers are indicated at left. Results are representative of at least three biological replicates. FIG. 1C shows representative size exclusion chromatography (SEC) analysis of Protein A/G purified ch735. Antibody was analyzed on a 4.6 mm ID×30 cm TSKgel SuperSW3000 SEC column with 4 μm particles. Pure antibody eluted with 0.1 M Na₂SO₄+0.1 M PBS pH 6.7 at 8.66 min.

FIGS. 2A-2D depict binding specificity and affinity of the ch735 antibody. FIG. 2A shows antigen binding activity for recombinant purified ch735 determined by ELISA with either NCAM or endoN-treated NCAM immobilized as antigens. ELISA signals (Abs₃₇₀) were obtained with anti-human IgG secondary antibodies. FIG. 2B shows glycoprotein-binding specificity of ch735 probed using an array of ˜50 glycoproteins. Antibodies were assayed at 1 μg/mL and detected with anti-human IgG secondary antibodies. FIG. 2C shows glycan-binding specificity of ch735 measured against CFG glycan microarray (version 5.4) that contained ˜585 natural and synthetic mammalian glycans (http://www.functionalglycomics.org). Antibodies were assayed at 10 μg/mL and detected with anti-mouse IgG secondary antibodies. All data are the average of three replicate experiments and error bars are the standard deviation of the mean. FIG. 2D shows the glycan-binding specificity as in FIG. 2C, but only showing data for glycan structures containing α2,8-linked sialic acid (see Table 1 for a list of the corresponding structures).

FIGS. 3A-3D depict binding specificity and affinity of mo735 antibody. FIG. 3A shows antigen binding activity for commercial mo735 determined by ELISA with either NCAM or endoN-treated NCAM immobilized as antigen. ELISA signals (Abs₃₇₀) were obtained with anti-mouse IgG secondary antibodies. FIG. 3B shows glycoprotein-binding specificity of mo735 probed using an array of ˜50 glycoproteins. Antibodies were assayed at 1 μg/mL and detected with anti-mouse IgG secondary antibodies. FIG. 3C shows glycan-binding specificity of mo735 measured against CFG glycan microarray version 5.3 that contained ˜600 natural and synthetic mammalian glycans. Antibodies were assayed at 10 μg/mL and detected with anti-mouse IgG secondary antibodies. All data are the average of three replicate experiments and error bars are the standard deviation of the mean. FIG. 3D shows glycan-binding specificity as in FIG. 3D, but only showing data for glycan structures containing α2,8-linked sialic acid (see Table 1 for a list of the corresponding structures).

FIGS. 4A-4B depict confirmation of NCAM and endoN-treated NCAM coating of ELISA plates. FIG. 4A shows antigen binding activity for commercial antibody ab5032 specific for NCAM determined by ELISA with either NCAM or endoN-treated NCAM as immobilized antigen. FIG. 4B shows antigen binding activity as in FIG. 4A, but with commercial antibody ch735 instead of NCAM-specific ab5032 antibody. ELISA signals (Abs₄₅₀) were obtained with anti-mouse IgG secondary antibodies.

FIGS. 5A-5D depict binding affinity analysis of mo735 and ch735 antibodies. Binding kinetics for the mo735 and ch735 antibodies were monitored using Biacore. Commercial mo735 or recombinant purified ch735 was immobilized at low concentrations on a Biacore Protein A sensor chip and the response to different concentrations of NCAM (ranging from 0.25-250 nM) was compared with an empty flow cell. FIGS. 5A-5B depicts representative sensorgram data for mo735 (FIG. 5A) and ch735 (FIG. 5B). FIG. 5C shows the data evaluated by plotting maximum binding signal against NCAM concentration (•) with binding curve calculated using the Hill slope non-linear regression analysis in Prism (−). The calculated K_{d} values from the Hill slope analysis are given in FIG. 5D.

FIGS. 6A-6D depict immunostaining of antibody ch735 to polySia-expressing cancer cells. FIG. 6A shows external levels of polySia on a panel of cancer cell lines with or without endoN treatment measured by flow cytometry using ch735 and fluorescent anti-human secondary. Data are the geometric mean fluorescence intensity (MFI), with the values reported as the average of three replicates and the error represented as the standard deviation of the mean. FIG. 6B shows confocal microscopic images of endoN-treated and non-treated polySia expressing cancer cell lines to assess ch735 binding on the cell surface. Cells were stained with ch735, wheat germ agglutinin (WGA) to stain the cell membrane, and Hoescht to stain nuclei. Scale bars, 10 μm. FIG. 6C shows formalin-fixed, paraffin-embedded (FFPE) human tissue sections of SCLC and FIG. 6D shows adjacent normal tissue stained for polySia with mo735. Scale bars, 200 μm.

FIGS. 7A-7B depict binding of antibody ch735 and mo735 to polySia-expressing cancer cells. Representative fluorescence histograms for ch735 (FIG. 7A) and mo735 (FIG. 7B) binding to a panel of cancer cell lines measured by flow cytometry are shown. Histograms represent antibody binding to untreated cells, antibody binding to endoN-treated cells, and anti-human or anti-mouse secondary only control, respectively.

FIGS. 8A-8C depict binding of polySia-specific mAb to ST8SiaII and ST8SiaIV knockout cell lines. FIG. 8A shows external levels of polySia on wild-type (wt) SW2 cells and ST8SiaII and ST8SiaIV CRISPR/Cas9 knockout (KO) cell lines measured by flow cytometry using mo735 and fluorescent anti-mouse secondary. Data are the geometric mean fluorescence intensity (MFI), with the values reported as the average of three replicates and the error represented as the standard deviation of the mean. FIG. 8B shows histograms of representative samples of antibody binding to wild-type SW2 cells, ST8SiaII KO SW2 cells, and anti-mouse secondary only control. In the ST8SiaII KO SW2 cells, 73% of the population (FL1-H-) binds mo735 below the level of the wild-type SW2 cells. FIG. 8C shows histograms of representative samples of antibody binding to wild-type SW2 cells, ST8SiaIV KO SW2 cells, and anti-mouse secondary only control. In the ST8SiaIV KO SW2 cells, 68% of the population (FL1-H-) binds mo735 below the level of the wild-type SW2 cells.

FIGS. 9A-9B depict binding of antibody mo735 to polySia-expressing cancer cells. FIG. 9A shows external levels of polySia on a panel of cancer cell lines with or without endoN treatment measured by flow cytometry using mo735 and fluorescent anti-mouse secondary. Data are the geometric mean fluorescence intensity (MFI), with the values reported as the average of three replicates and the error represented as the standard deviation of the mean. FIG. 9B shows confocal microscopic images of endoNtreated and non-treated polySia expressing cancer cell lines to assess mo735 binding on the cell surface. Cells were stained with ch735, wheat germ agglutinin (WGA), and nuclei were stained with Hoescht. Scale bars, 10 μm.

FIGS. 10A-10E depict internalization of ch735 into polySia-positive cancer cells. FIG. 10A shows internalization of ch735 in polySia-positive cell lines SH-SY5Y, SW2, H69, and H82, and in polySia-negative MCF7 cells after 1 h. Data are reported as the mean percent internalization and error bars are the standard deviation of the mean (n=3). FIG. 10B shows a time course of antibody internalization in polySia-positive cell line SH-SY5Y treated with ch735 or isotype control. Data reported as the mean percent internalization and error bars are the standard deviation of the mean (n=3). FIG. 10C shows confocal microscopy images of SH-SY5Y cells incubated for 1 h with ch735 labeled with AF488 and transferrin labeled with AF647. Nuclei were stained by Hoescht (blue). Scale bar, 10 Fluorescence intensity was measured across the dotted line and normalized to the maximum value in each channel. Arrows indicate regions of colocalization. The inset shows only the ch735 and DNA channels of the boxed region. FIG. 10D shows confocal microscopy images of SH-SY5Y cells incubated for 1 h with ch735 labeled with AF488 and anti-LAMP-3 labeled with AF647. Nuclei were stained by Hoescht. Scale bar, 10 Fluorescence intensity was measured across the dotted line and normalized to the maximum value in each channel. Arrows indicate regions of colocalization. The inset show only the ch735 and DNA channels of the boxed region. FIG. 10E shows confocal microscopy images of SH-SY5Y cells incubated for 120 min with ch735. Lysosomes were stained with anti-LAMP-1 and A647-labeled anti-rabbit antibody, ch735 was stained with AF488-labeled anti-human antibody, and nuclei were stained by Hoescht. Scale bar, 10 Fluorescence intensity was measured across the dotted white line and normalized to the maximum value in each channel. Arrows indicate regions of colocalization. The top right inset shows only the ch735 and DNA channels of the boxed region.

FIGS. 11A-11D depict internalization of mo735 by polySia-expressing cancer cells. FIG. 11A shows intracellular fluorescence of SW2 cells following incubation for 180 min with various concentrations of mo735 labeled with AF488 measured by flow cytometry. Trypan blue was used to quench extracellular fluorescence. FIG. 11B shows confocal microscopic images of SW2 cells incubated with mo735 labeled with pHrodo Green for t=0 and t=40 min. Cell membranes were labeled with wheat germ agglutinin (WGA,) and nuclei were stained with Hoescht. Scale bar, 10 μm. FIG. 11C shows confocal microscopic images of SW2 cells with or without endoN treatment incubated with mo735 or isotype labeled with AF488 for 1 h. Cell membranes were labeled with wheat germ agglutinin (WGA) and nuclei were stained with Hoescht. Scale bar, 10 μm. FIG. 11D shows confocal microscopic images of SW2 cells incubated with mo735 for t=0, 1, and 4 h. Images include external mo735 detected with anti-mouse AF488, internal mo735 detected with anti-mouse AF647, nuclei stained with Hoescht, and a merged image. Scale bar, 10 μm.

FIGS. 12A-12C depict colocalization of mo735 with markers that traffic to endolysosomal compartments. FIG. 12A shows confocal microscopic images taken of SH-SY5Y cells incubated with ch735 (top panels) or isotype antibody (bottom panels) labeled with AF488 and transferrin labeled with AF647 for 1 h (left) and anti-LAMP-3 labeled with AF647 for 1 h (middle). For LAMP-1 (right), confocal microscopic images were taken after labeling SH-SY5Y cells with ch735 or isotype antibody for 2 h. Following fixation and permeabilization, LAMP-1 antibody was applied and detected with anti-rabbit AF647 secondary. Anti-human IgG AF488 was used to detect ch735 or isotype antibody. FIG. 12B shows confocal microscopic images taken of SW2 cells incubated for 30 min with mo735 labeled with AF488 and transferrin labeled with AF647 (red). FIG. 12C shows confocal microscopic images taken of SW2 cells incubated with mo735 labeled with AF488 and anti-LAMP-3 antibody labeled with AF647 for 30 min. Cross-sectional fluorescence profiles are shown at right with normalized pixel intensity for selected cells (marked with box in left confocal panels) versus distance across the cell for representative cross sections (marked with bar in right confocal panels).

FIGS. 13A-13D depict target-mediated in vitro cytotoxicity of glycan-directed ADC. FIG. 13A shows an overview of the two-step ADC synthesis strategy used to generate ch735-Py-DM1. The first step involved conjugation of NHS-PEG4-tetrazine (NHS-Tz) to free lysines and the second step involved the reaction of the trans-cyclooctene (TCO) group on the TCO-maleimide-DM1 drug linker (TCO-mal-DM1) with the Tz on the antibody. FIG. 13B shows the chemical structure of the non-cleavable drug linker with DM1. FIG. 13C shows the viability of SH-SY5Y (polySia+) and MCF7 (polySia-) cells following treatment with different concentrations of ch735-Py-DM1 or isotype-Py-DM1. Percent viability is calculated based on the signal relative to untreated control cells. Representative data depicts mean percent viability and error bars are the standard deviation of the mean (n=3). FIG. 13D shows viability of SKOV3 (HER2+) and SH-SY5Y (HER2-) cells following treatment with different concentrations of T-Py-DM1 and isotype-Py-DM1. Representative data depicts mean percent viability and error bars are the standard deviation of the mean (n=3).

FIGS. 14A-14C depict synthesis and characterization of TCO-maleimide-DM1 non-cleavable drug linker. FIG. 14A shows the chemical structure and chemical synthetic route for TCO-maleimide-DM1 non-cleavable drug linker: (i) 3:1 DMSO:PBS (pH 7.4) at 37° C. overnight. FIG. 14B shows absorbance profile of purified TCO-maleimide-DM1 linker at 260 nm and FIG. 14C shows LC/MS characterization of TCO-maleimide-DM1 linker. Expected mass=1260.56; observed M+H=1261.500; observed M+Na=1283.400.

FIG. 15 depicts cellular internalization of a polySia-specific single-chain Fv (scFv735) constructed by genetically fusing together the DNA encoding the variable heavy (V_{H}) and variable light (V_{L}) genes derived from mo735 with a flexible GlySer linker. Akin to the results with the mAb, scFv735 was observed to internalize in SW2 cells that express polySia on their surface but not MCF7 cells that do not display the polySia antigen on their surface. Internalization was observed to increase with increasing concentration of scFv735 and was blocked when the temperature was reduced to 4° C., indicating an endocytic mechanism.

FIGS. 16A-16G depict the nucleic acid sequence (SEQ ID NO: 15) and features of the plasmid pVITRO-735-IgG1/k, which encodes the ch735 antibody.

FIGS. 17A-17G depict the nucleic acid sequence (SEQ ID NO: 16) and features of the plasmid pVITRO1-Trastuzumab-IgG1/k, which encodes the trastuzumab antibody.

### DETAILED DESCRIPTION

Unless otherwise indicated, the definitions and embodiments described in this and other sections are intended to be applicable to all embodiments and aspects of the present application herein described for which they are suitable as would be understood by a person skilled in the art.

As used in this application, the singular forms “a”, “an” and “the” include plural references unless the content clearly dictates otherwise.

The term “and/or” as used herein means that the listed items are present, or used, individually or in combination. In effect, this term means that “at least one of” or “one or more” of the listed items is used or present.

Where a range of values is provided, it is intended that each intervening value between the upper and lower limit of that range and any other stated or intervening value in that stated range is encompassed within the disclosure. For example, if a range of 1 to 10 minutes is stated, it is intended that 2 minutes, 3 minutes, 4 minutes, 5 minutes, 6 minutes, 7 minutes, 8 minutes, and 9 minutes are also explicitly disclosed, as well as the range of values greater than or equal to 1 minute and the range of values less than or equal to 10 minutes.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

One aspect of the present application is an immunoconjugate therapeutic comprising a polysialic acid targeting portion and an anti-cancer therapeutic coupled to the polysialic acid targeting portion.

As used herein, the term “polysialic acid,” (also referred to herein as “polySia” and “PSA”) refers to the glycan homopolymer of α2,8-linked N-acetyl neuraminic acid (NeuNAc).

As used herein, the term “targeting portion” refers to a component that is able to bind to or otherwise associate with a molecular target, for example, a membrane component, a cell surface receptor, polysialic acid, or the like. The targeting portion may become localized or converge at a particular targeted site, for instance, a tumor, a disease site, a tissue, an organ, a type of cell, etc. As such, the targeting portion may be “target-specific” and can be said to target, for example, a particular type of cell, such a polysialic acid positive tumor cell.

For example, a targeting portion may include a nucleic acid, peptide, polypeptide, protein, glycoprotein, carbohydrate, or lipid. A targeting portion may be a naturally occurring or synthetic ligand for a cell surface receptor, e.g., a growth factor, hormone, LDL, transferrin, etc. A targeting component can be an antibody, which term is intended to include antibody fragments, characteristic portions of antibodies, single chain targeting moieties which can be identified, for example, using procedures such as phage display. Targeting components may also be a targeting peptide, targeting peptidomimetic, or a small molecule, whether naturally-occurring or artificially created (e.g., via chemical synthesis).

In one embodiment, the targeting portion is a polysialic acid targeting portion. In a further embodiment, the specificity of the polysialic acid targeting portion is for α2,8-linked sialic acid with a degree of polymerization (DP) of three or greater.

In another embodiment, the polysialic acid targeting portion is a mammalian polysialic acid targeting portion; i.e., the polysialic acid targeting portion targets mammalian-expressed polysialic acid. In a further embodiment, the polysialic acid targeting portion is a human polysialic acid targeting portion; that is, the targeting portion targets human-expressed polysialic acid.

In one embodiment, the polysialic acid targeting portion is an antibody.

Antibodies of the embodiments of the present application may exist in a variety of forms including, for example, polyclonal antibodies, monoclonal antibodies, intracellular antibodies (“intrabodies”), antibody fragments (e.g. Fv, Fab and F(ab)2), half-antibodies, hybrid derivatives, as well as single chain antibodies (scFv), chimeric antibodies and humanized antibodies (Ed Harlow and David Lane, USING ANTIBODIES: A LABORATORY MANUAL (Cold Spring Harbor Laboratory Press, 1999); Houston et al., “Protein Engineering of Antibody Binding Sites: Recovery of Specific Activity in an Anti-Digoxin Single-Chain Fv Analogue Produced in Escherichia coli,” Proc. Natl. Acad. Sci. USA 85:5879-5883 (1988); and Bird et al, “Single-Chain Antigen-Binding Proteins,” Science 242:423-426 (1988), which are hereby incorporated by reference in their entirety).

Antibodies of the embodiments of the present application may also be synthetic antibodies. A synthetic antibody is an antibody which is generated using recombinant DNA technology, such as, for example, an antibody expressed by a bacteriophage. Alternatively, the synthetic antibody is generated by the synthesis of a DNA molecule encoding and expressing the antibody of the present application or the synthesis of an amino acid sequence specifying the antibody, where the DNA or amino acid sequence has been obtained using synthetic DNA or amino acid sequence technology which is available and well known in the art.

Methods for monoclonal antibody production may be carried out using the techniques described herein or are well-known in the art (MONOCLONAL ANTIBODIES—PRODUCTION, ENGINEERING AND CLINICAL APPLICATIONS (Mary A. Ritter and Heather M. Ladyman eds., 1995), which is hereby incorporated by reference in its entirety). Generally, the process involves obtaining immune cells (lymphocytes) from the spleen of a mammal which has been previously immunized with the antigen of interest either in vivo or in vitro.

Alternatively, monoclonal antibodies can be made using recombinant DNA methods as described in U.S. Pat. No. 4,816,567 to Cabilly et al, which is hereby incorporated by reference in its entirety. The polynucleotides encoding a monoclonal antibody are isolated from mature B-cells or hybridoma cells, for example, by RT-PCR using oligonucleotide primers that specifically amplify the genes encoding the heavy and light chains of the antibody. The isolated polynucleotides encoding the heavy and light chains are then cloned into suitable expression vectors, which when transfected into host cells such as E. coli cells, simian COS cells, Chinese hamster ovary (CHO) cells, or myeloma cells that do not otherwise produce immunoglobulin protein, monoclonal antibodies are generated by the host cells. Also, recombinant monoclonal antibodies or fragments thereof of the desired species can be isolated from phage display libraries (McCafferty et al., “Phage Antibodies: Filamentous Phage Displaying Antibody Variable Domains,” Nature 348:552-554 (1990); Clackson et al., “Making Antibody Fragments using Phage Display Libraries,” Nature 352:624-628 (1991); and Marks et al., “By-Passing Immunization. Human Antibodies from V-Gene Libraries Displayed on Phage,” J. Mol. Biol. 222:581-597 (1991), which are hereby incorporated by reference in their entirety).

The polynucleotide(s) encoding a monoclonal antibody can further be modified using recombinant DNA technology to generate alternative antibodies. For example, the constant domains of the light and heavy chains of a mouse monoclonal antibody can be substituted for those regions of a human antibody to generate a chimeric antibody. Alternatively, the constant domains of the light and heavy chains of a mouse monoclonal antibody can be substituted for a non-immunoglobulin polypeptide to generate a fusion antibody. In other embodiments, the constant regions are truncated or removed to generate the desired antibody fragment of a monoclonal antibody. Furthermore, site-directed or high-density mutagenesis of the variable region can be used to optimize specificity and affinity of a monoclonal antibody.

The monoclonal antibody of the embodiments of the present application can be a humanized antibody. Humanized antibodies are antibodies that contain minimal sequences from non-human (e.g., murine) antibodies within the variable regions. Such antibodies are used therapeutically to reduce antigenicity and human anti-mouse antibody responses when administered to a human subject. In practice, humanized antibodies are typically human antibodies with minimal to no non-human sequences. A human antibody is an antibody produced by a human or an antibody having an amino acid sequence corresponding to an antibody produced by a human.

In addition to whole antibodies, the embodiments of the present application encompasses binding portions of such antibodies. Such binding portions include the monovalent Fab fragments, Fv fragments (e.g., single-chain antibody, scFv), and single variable V_{H} and V_{L} domains, and the bivalent F(ab′)2 fragments, Bis-scFv, diabodies, triabodies, minibodies, etc. These antibody fragments can be made by conventional procedures, such as proteolytic fragmentation procedures, as described in James Goding, MONOCLONAL ANTIBODIES: PRINCIPLES AND PRACTICE 98-118 (Academic Press, 1983) and Ed Harlow and David Lane, ANTIBODIES: A LABORATORY MANUAL (Cold Spring Harbor Laboratory, 1988), which are hereby incorporated by reference in their entirety, or other methods known in the art.

It may further be desirable, especially in the case of antibody fragments, to modify the antibody in order to increase its serum half-life. This can be achieved, for example, by incorporation of a salvage receptor binding epitope into the antibody fragment by mutation of the appropriate region in the antibody fragment or by incorporating the epitope into a peptide tag that is then fused to the antibody fragment at either end or in the middle (e.g., by DNA or peptide synthesis).

Antibody mimics are also suitable for use in accordance with the present application. A number of antibody mimics are known in the art including, without limitation, those known as monobodies, which are derived from the tenth human fibronectin type III domain (¹⁰Fn3) (Koide et al., “The Fibronectin Type III Domain as a Scaffold for Novel Binding Proteins,” J. Mol. Biol. 284:1141-1151 (1998); Koide et al., “Probing Protein Conformational Changes in Living Cells by Using Designer Binding Proteins: Application to the Estrogen Receptor,” Proc. Natl. Acad. Sci. USA 99:1253-1258 (2002), each of which is hereby incorporated by reference in its entirety); and those known as affibodies, which are derived from the stable alpha-helical bacterial receptor domain Z of staphylococcal protein A (Nord et al., “Binding Proteins Selected from Combinatorial Libraries of an alpha-helical Bacterial Receptor Domain,” Nature Biotechnol. 15(8):772-777 (1997), which is hereby incorporated by reference in its entirety).

In an embodiment, the targeting portion is a full length immunoglobulin; in other embodiments, the targeting portion is a binding portion thereof. In an embodiment, the full length immunoglobulin, or portion thereof, is selected from a single chain variable fragment (scFv), a single chain antibody fragment (scab), a single domain antibody (dAb), a fragment antigen binding (Fab) fragment, a Fab′ fragment, F(ab′)2 fragment, a single-chain Fv fused to Fc domain (scFv-Fc), a single domain antibody fused to Fc domain (dAb-Fc), a free light chain (free LC), a half antibody, wherein the targeting portion binds to the target of interest, such as polysialic acid.

In one embodiment, the targeting portion is a monoclonal antibody. In a further embodiment, the targeting portion is a polyclonal antibody.

In another embodiment, the targeting portion is a mouse antibody, a human antibody, a chimeric antibody, or a humanized antibody.

In an additional embodiment, the targeting portion is monoclonal antibody mo735.

In another embodiment, the targeting portion is a derivative of monoclonal antibody mo735, which binds to polysialic acid.

In a further embodiment, the targeting portion is monoclonal antibody ch735.

In another embodiment, the targeting portion is a derivative of monoclonal antibody ch735, which binds to polysialic acid.

In one embodiment, the polysialic acid targeting portion comprises a light chain variable region and a heavy chain variable region.

In another embodiment, the polysialic acid targeting portion is an antibody comprising a light chain variable region and a heavy chain variable region, wherein the light chain variable region has an amino acid sequence comprising SEQ ID NO: 1, as follows:

MTQTPLSLPVSLGDQASISCRSSQSLVHSNGNTYLYWYLQKPGQSPKPLI
YRVSNRFSGVPDRFSGSGSGTDFTLKISRVEAEDLGVYFCFQGTHVPYTF
GGGTRLEIK,

and the heavy chain region has an amino acid sequence comprising SEQ ID NO: 2, as follows:

QIQLQQSGPELVRPGASVKISCKASGYTFTDYYIHWVKQRPGEGLEWIGW
IYPGSGNTKYNEKFKGKATLTVDTSSSTAYMQLSSLTSEDSAVYFCARGG
KFAMDYWGQGTSVTVSS.

In an additional embodiment, the polysialic acid targeting portion is an antibody comprising a light chain variable region and a heavy chain variable region, wherein the light chain variable region is encoded by a nucleic acid sequence comprising SEQ ID NO: 3, as follows:

GATGTAGTCATGACGCAGACGCCACTTAGCTTACCGGTCAGTTTAGGGGA
TCAGGCGAGCATTAGCTGTCGCTCCTCACAGAGCTTGGTTCACAGCAATG
GGAACACGTACCTGTACTGGTATCTGCAGAAACCGGGCCAATCGCCGAAA
CCGCTCATCTATCGGGTATCGAATCGCTTTAGTGGGGTTCCCGATCGCTT
TTCTGGTTCTGGATCGGGGACAGACTTCACTCTGAAGATTAGCCGCGTTG
AAGCCGAAGATCTGGGCGTGTACTTCTGCTTTCAAGGGACGCATGTGCCG
TATACCTTTGGCGGTGGGACTCGCCTGGAAATCAAA,

and the heavy chain region his encoded by a nucleic acid sequence comprising SEQ ID NO: 4, as follows:

CAGATTCAGCTGCAGCAATCTGGTCCAGAGCTTGTTCGTCCTGGCGCATC
AGTGAAAATCTCGTGCAAAGCATCCGGTTACACCTTTACGGACTATTACA
TCCATTGGGTGAAACAACGTCCTGGTGAAGGTTTGGAATGGATTGGTTGG
ATTTATCCGGGCAGCGGCAACACCAAGTATAACGAGAAGTTCAAAGGCAA
AGCCACTCTCACCGTGGATACATCGTCCAGCACCGCTTACATGCAGCTGA
GTTCTCTGACCTCTGAAGATTCCGCGGTCTATTTCTGTGCTCGTGGTGGC
AAATTTGCGATGGACTATTGGGGCCAAGGCACCAGCGTAACCGTGTCATC
C.

In one embodiment, the polysialic acid targeting portion comprises multiple binding sites to its molecular target. In another embodiment, the polysialic acid targeting portion is biotinylated and cross-linked to additional polysialic acid targeting portions.

As used herein, the term “cancer” includes all types of cancerous growths or oncogenic processes, metastatic tissues or malignantly transformed cells, tissues, or organs, irrespective of histopathologic type or stage of invasiveness.

As used herein, the term “anti-cancer therapeutic” refers to an effector molecule that provides an anti-cancer effect. Anti-cancer therapeutics have various mechanisms of action and may include molecules with cytostatic effects as well as molecules with cytotoxic effects.

In one embodiment, the anti-cancer therapeutic may be a pharmacological agent, a radionucleotide, or a catalytic toxin.

In another embodiment, the anti-cancer therapeutic may be a microtubule disrupting agent, a DNA modifying agent, a RNA modifying agent, a DNA damaging agent, or a RNA damaging agent.

In a further embodiment, the anti-cancer therapeutic may be a maytansinoid, an auristatin, a tubulysin, a duocarymycin, a calicheamicin, a pyrrolobenzodiazepine, a radionuclide, an amatoxin, a camptothecin, a doxorubicin, a 5-fluorouracil, or a methotrexate.

In another embodiment, the anti-cancer therapeutic is a mayatansinoid selected from emtansine (DM1) and ravtansine (DM4). In an additional embodiment, the mayatansinoid is emtansine (DM1).

In one embodiment, the immunoconjugate therapeutic comprises more than one anti-cancer therapeutic coupled to the polysialic targeting portion.

In some cases, the anti-cancer therapeutic may exert its anti-cancer effect without the need for release from the targeting portion. In other cases, the anti-cancer therapeutic may be released from the targeting portion and allowed to interact locally with the particular targeting site.

As used herein, the term “coupled” refers to attachment by covalent bonds or by strong non-covalent interactions. Any method normally used by those skilled in the art for the coupling of biologically active materials can be used.

In an embodiment, the targeting portion is coupled to the anti-cancer therapeutic through a linker element.

As used herein, the term “linker element” refers to a linking moiety that connects two groups. A linker element can be a molecule or sequence, such as an amino acid sequence, that attaches, as in a bridge, one molecule or sequence to another molecule or sequence. “Linked,” “conjugated,” or “coupled” means attached or bound by covalent bonds, or non-covalent bonds, or other bonds, such as van der Waals forces.

In an embodiment, the linker is either a cleavable linker or a non-cleavable linker.

As used herein, the term “cleavable linker” refers to a linker that can be selectively cleaved to produce two products. Application of suitable cleavage conditions to a molecule containing a cleavable linker that is cleaved by the cleavage conditions will produce two cleavage products. A cleavable linker of the present application is stable, e.g. to physiological conditions, until it is contacted with a reagent capable of cleaving the cleavable linker.

As used herein, the term “non-cleavable linker” refers to inkers that rely on lysosomal degradation to release the anti-cancer therapeutic from the targeting portion of the immunoconjugate therapeutic.

Various linker elements and conjugation chemistries suitable for use in the present application are known and described in the art, e.g., McCombs et al., “Antibody Drug Conjugates: Design and Selection of Linker, Payload and Conjugation Chemistry,” AAPS J. 17(2):339-351 (2015); and Tsuchikama et al., “Antibody-Drug Conjugates: Recent Advances in Conjugation and Linker Chemistries,” Protein Cell 9(1):33-46 (2018), which are hereby incorporated by reference in their entirety.

In one embodiment, the linker element is capable of being synthesized via a bioorthogonal conjugation reaction.

In a further embodiment, the linker element comprises a conjugation moiety element capable of synthesis via a bioorthogonal conjugation reaction.

In another embodiment, the linker element comprises a pyradizine-containing conjugation moiety. In an additional embodiment, the linker element comprises a 1,4-dihydropyridazine (Py) containing conjugation moiety, where 1,4-dihydropyradizine is represented by the formula:

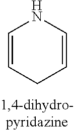

As used herein, the term “bioorthogonal conjugation reaction” refers to a reaction that does not interfere with native biochemical processes inside living systems. Bioorthogonal conjugation reactions include the Staudinger ligation, the azide-cyclooctyne cycloaddition, and the inverse-electron-demand Diels-Alder reaction.

In one embodiment, the linker element comprises a conjugation moiety capable of being synthesized via a bioorthogonal conjugation reaction between a tetrazine and a transcyclooctene (TCO). A bioorthogonal conjugation reaction between a tetrazine and a TCO is represented by the formula:

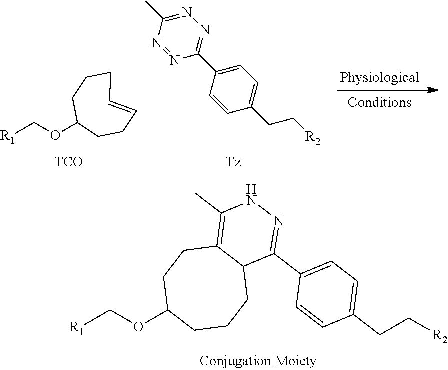

where R1 and R2 each independently comprises a reaction group capable of binding to an element such as the target portion or the anti-cancer therapeutic, and may also comprise additional linker components, such as, for example, spacers.

In an embodiment, the linker element comprises a hydrophilic group or groups. In an embodiment, the hydrophilic group is a polyethylene glycol (PEG) chain.

Hydrophilic linkers are described, for example, in Walker et al., “Hydrophilic Sequence-Defined Cross-Linkers for Antibody-Drug Conjugates,” Bioconjugate Chemistry 30:2982-2988 (2019), which is hereby incorporated by reference in its entirety.

In one embodiment, the linker element comprises one or more spacer arms. In one embodiment, the spacer arm is a polyethylene glycol (PEG) spacer arm. In an additional embodiment, the linker element comprises two PEG spacer arms. In another embodiment, the linker element comprises a conjugation moiety capable of being synthesized via a bioorthogonal conjugation reaction, and further comprises a PEG spacer arm between the targeting portion and the conjugation moiety. In a further embodiment, the linker element comprises a conjugation moiety capable of being synthesized via a bioorthogonal conjugation reaction, and further comprises a PEG spacer arm between the anti-cancer therapeutic and the conjugation moiety. In yet another embodiment, the linker element comprises a conjugation moiety capable of being synthesized via a bioorthogonal conjugation reaction, and further comprises a first PEG spacer arm between the targeting portion and the conjugation moiety and a second PEG spacer arm between the anti-cancer therapeutic and the conjugation moiety.

PEG spacer arms may comprise multiple PEG units (i.e. multiple O—CH₂—CH₂ units). In embodiments, the PEG spacer arm comprises from about 1 to 10, 1 to 9, 1 to 8, 1 to 7, 1 to 6, 1 to 5, 1 to 4, 1 to 3, 1 to 2, 2 to 10, 2 to 9 m, 2 to 8, 2 to 7, 2 to 6, 2 to 5, 2 to 4, 2 to 3, 3 to 10, 3 to 9, 3 to 8, 3 to 7, 3 to 6, 3 to 5, 3 to 4, 4 to 10, 5 to 10, 5 to 9, 5 to 8, 5 to 7, 5 to 6, 6 to 10, 6 to 9, 6 to 8, 6 to 7, 7 to 10, 7 to 9, 7 to 8, 8 to 10, 8 to 9, or 9 to 10 PEG units. In embodiments in which the linker element contains multiple PEG elements, each PEG element has a number of PEG units independent of the other PEG element(s) such that the number of PEG units of any one PEG element in a linker element may be the same as any of the other PEG element(s) in a linker element, or may different from any of the other PEG element(s) in a linker element.

In an embodiment, the linker element comprises a conjugation moiety capable of being synthesized via a bioorthogonal conjugation reaction, and further comprises a first PEG spacer arm comprising four PEG units, wherein the first PEG spacer arm is situated between the targeting portion and the conjugation moiety, and a second PEG spacer arm comprising three PEG units, wherein the second PEG spacer arm is situated between the anti-cancer therapeutic and the conjugation moiety.

In one embodiment, the linker element comprises:

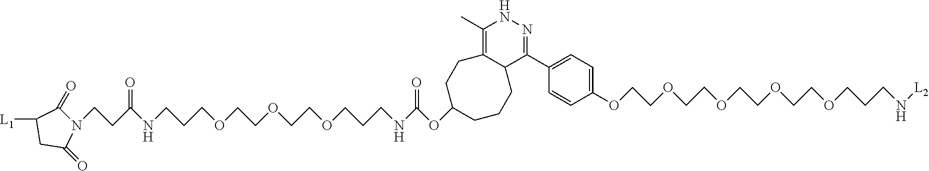

where L1 and L2 each independently comprises an element such as the target portion or the anti-cancer therapeutic, and may also comprise additional linker elements, such as, for example, spacer elements.

In one embodiment, the immunoconjugate therapeutic has the formula:

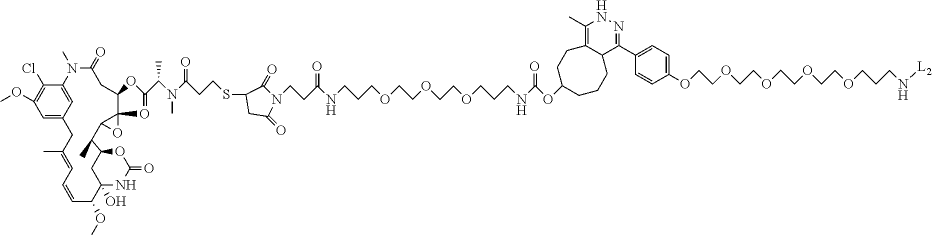

where L2 is a polysialic acid targeting portion, such as ch735 or a derivative thereof.

Another aspect of the present application is an immunoconjugate therapeutic with the formula:

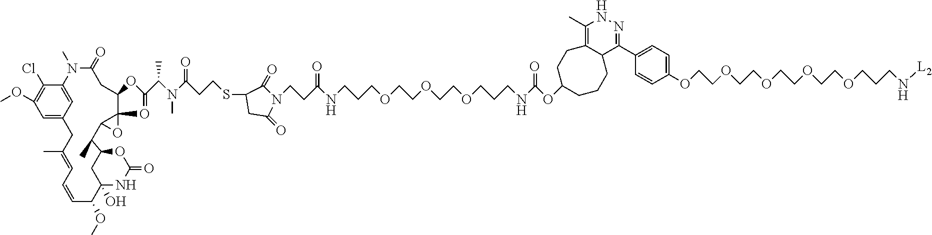

where L2 is a HER-2 targeting portion, such as trastuzumab or a derivative thereof.

In one embodiment, the immunoconjugate therapeutic is internalized into a targeted cell or cell population. In one embodiment, the immunoconjugate therapeutic is internalized into the endosomal compartment. In another embodiment, the immunoconjugate therapeutic is internalized into the lysosomal compartment. Internalization into a particular cellular compartment can also be characterized by percent internalization, as a function of time.

In one embodiment, percent internalization is in a range of from about 10% to 100%, 10% to 80%, 10% to 60%, 10% to 50%, 10% to 40%, 10% to 30%, 10% to 20%, 20% to 80%, 20% to 60%, 20% to 50%, 20% to 40%, 20% to 30%, 30% to 80%, 30% to 60%, 30% to 50%, 30% to 40%, 40% to 80%, 40% to 60%, 40% to 50%, 50% to 80%, 50% to 60%, or 60% to 80%. In an additional embodiment, the percent internalization is achieved at an amount of time after administering the immunoconjugate therapeutic in a range in minutes from about 1 to 120, 1 to 90, 1 to 60, 1 to 45, 1 to 30, 1 to 15, 1 to 10, 1 to 5, 1 to 4, 1 to 3, 1 to 2, 5 to 30, 5 to 15, 5 to 10, 10 to 30, 10 to 15, or 15 to 30.

The immunoconjugate therapeutics of the present application can be characterized by the average number of drug modules (i.e., cytotoxic agents) of each antibody in the molecule, i.e. the Drug-to-Antibody Ratio (DAR). The DAR values affect the efficacy of the drug (Sun et al., “Effects of Drug-Antibody Ratio on Pharmacokinetics, Biodistribution, Efficacy, and Tolerability of Antibody-Maytansinoid Conjugates,” Bioconjugate Chem. 28(5):1371-1381 (2017), which is hereby incorporated by reference in its entirety). The DAR can be verified by conventional means, such as mass spectrometry, ELISA assay, and HPLC.

In one embodiment, the immunoconjugate therapeutic has a DAR in a range of from about 2 to 8, 2 to 7, 2 to 6, 2 to 5, 2 to 4, 2 to 3, 3 to 8, 3 to 7, 3 to 6, 3 to 5, 3 to 4, 4 to 8, 4 to 7, 4 to 6, 4 to 5, 5 to 8, 5 to 7, 5 to 6, 6 to 8, 6 to 7, or 7 to 8.

The immunoconjugate therapeutics of the present application can be characterized by potency, which can be measured as EC50, which is defined as the concentration which induces a response halfway between the baseline and maximum after a specified exposure time. Methods of measuring EC50 are known in the art and described, for example, in Sebaugh, J L., “Guidelines for Accurate EC50/IC50 Estimation,” Pharmaceutical Statistics 10(2):128-134 (2011), which is hereby incorporated by reference in its entirety.

In one embodiment, the immunoconjugate therapeutic has an EC50 (in nM) in a range from about 0.00001 to 10,000, 0.00001 to 9,000, 0.00001 to 8,000, 0.00001 to 7,000, 0.00001 to 6,000, 0.00001 to 5,000, 0.00001 to 4,000, 0.00001 to 3,000, 0.00001 to 2,000, 0.00001 to 1,000, 0.00001 to 900, 0.00001 to 800, 0.00001 to 700, 0.00001 to 600, 0.00001 to 500, 0.00001 to 400, 0.00001 to 300, 0.00001 to 200, 0.00001 to 100, 0.00001 to 90, 0.00001 to 80, 0.00001 to 70, 0.00001 to 60, 0.00001 to 50, 0.00001 to 40, 0.00001 to 30, 0.00001 to 25, 0.00001 to 20, 0.00001 to 15, 0.00001 to 10, 0.00001 to 5, 0.00001 to 4, 0.00001 to 3, 0.00001 to 2, 0.00001 to 1, 0.00001 to 0.1, 0.00001 to 0.01, 0.00001 to 0.001, 0.00001 to 0.0001, 0.00005 to 10,000, 0.00005 to 9,000, 0.00005 to 8,000, 0.00005 to 7,000, 0.00005 to 6,000, 0.00005 to 5,000, 0.00005 to 4,000, 0.00005 to 3,000, 0.00005 to 2,000, 0.00005 to 1,000, 0.00005 to 900, 0.00005 to 800, 0.00005 to 700, 0.00005 to 600, 0.00005 to 500, 0.00005 to 400, 0.00005 to 300, 0.00005 to 200, 0.00005 to 100, 0.00005 to 90, 0.00005 to 80, 0.00005 to 70, 0.00005 to 60, 0.00005 to 50, 0.00005 to 40, 0.00005 to 30, 0.00005 to 25, 0.00005 to 20, 0.00005 to 15, 0.00005 to 10, 0.00005 to 5, 0.00005 to 4, 0.00005 to 3, 0.00005 to 2, 0.00005 to 1, 0.00005 to 0.1, 0.00005 to 0.01, 0.00005 to 0.001, 0.00005 to 0.0001, 0.0001 to 10,000, 0.0001 to 1,000, 0.0001 to 100, 0.0001 to 90, 0.0001 to 80, 0.0001 to 70, 0.0001 to 60, 0.0001 to 50, 0.0001 to 40, 0.0001 to 30, 0.0001 to 25, 0.0001 to 20, 0.0001 to 15, 0.0001 to 10, 0.0001 to 5, 0.0001 to 4, 0.0001 to 3, 0.0001 to 2, 0.0001 to 1, 0.0001 to 0.1, 0.0001 to 0.01, 0.0001 to 0.001, 0.001 to 10,000, 0.001 to 1,000, 0.001 to 100, 0.001 to 90, 0.001 to 80, 0.001 to 70, 0.001 to 60, 0.001 to 50, 0.001 to 40, 0.001 to 30, 0.001 to 25, 0.001 to 20, 0.001 to 15, 0.001 to 10, 0.001 to 5, 0.001 to 4, 0.001 to 3, 0.001 to 2, 0.001 to 1, 0.001 to 0.1, 0.001 to 0.01, 0.01 to 10,000, 0.01 to 1,000, 0.01 to 100, 0.01 to 90, 0.01 to 80, 0.01 to 70, 0.01 to 60, 0.01 to 50, 0.01 to 40, 0.01 to 30, 0.01 to 25, 0.01 to 20, 0.01 to 15, 0.01 to 10, 0.01 to 5, 0.01 to 4, 0.01 to 3, 0.01 to 2, 0.01 to 1, 0.01 to 0.1, 0.1 to 10,000, 0.1 to 1,000, 0.1 to 100, 0.1 to 90, 0.1 to 80, 0.1 to 70, 0.1 to 60, 0.1 to 50, 0.1 to 40, 0.1 to 30, 0.1 to 25, 0.1 to 20, 0.1 to 15, 0.1 to 10, 0.1 to 5, 0.1 to 4, 0.1 to 3, 0.1 to 2, 0.1 to 1, 1 to 10,000, 1 to 1,000, 1 to 100, 1 to 90, 1 to 80, 1 to 70, 1 to 60, 1 to 50, 1 to 40, 1 to 30, 1 to 25, 1 to 20, 1 to 15, 1 to 10, 1 to 5, 1 to 4, 1 to 3, 1 to 2, 5 to 10,000, 5 to 1,000, 5 to 100, 5 to 90, 5 to 80, 5 to 70, 5 to 60, 5 to 50, 5 to 40, 5 to 30, 5 to 25, 5 to 20, 5 to 15, 5 to 10, 10 to 10,000, 10 to 1,000, 10 to 100, 10 to 90, 10 to 80, 10 to 70, 10 to 60, 10 to 50, 10 to 40, 10 to 30, 10 to 25, 10 to 20, 10 to 15, 15 to 10,000, 15 to 1,000, 15 to 100, 15 to 90, 15 to 80, 15 to 70, 15 to 60, 15 to 50, 15 to 40, 15 to 30, 15 to 25, 15 to 20, 20 to 10,000, 20 to 1,000, 20 to 100, 20 to 90, 20 to 80, 20 to 70, 20 to 60, 20 to 50, 20 to 40, 20 to 30, 20 to 25, 25 to 10,000, 25 to 1,000, 25 to 100, 25 to 90, 25 to 80, 25 to 70, 25 to 60, 25 to 50, 25 to 40, 25 to 30, 30 to 10,000, 30 to 1,000, 30 to 100, 30 to 90, 30 to 80, 30 to 70, 30 to 60, 30 to 50, 30 to 40, 40 to 10,000, 40 to 1,000, 40 to 100, 40 to 90, 40 to 80, 40 to 70, 40 to 60, 40 to 50, 50 to 10,000, 50 to 1,000, 50 to 100, 50 to 90, 50 to 80, 50 to 70, 50 to 60, 60 to 10,000, 60 to 1,000, 60 to 100, 60 to 90, 60 to 80, 60 to 70, 70 to 10,000, 70 to 1,000, 70 to 100, 70 to 90, 70 to 80, 80 to 10,000, 80 to 1,000, 80 to 100, 80 to 90, 90 to 10,000, 90 to 1,000, 90 to 100, 100 to 10,000, 100 to 1,000, or 1,000 to 10,000.

Another aspect of the present application is a method of treating subjects with cancer, said method comprising selecting a subject with cancer characterized by polysialic acid (polySia)-positive tumor cells and administering an immunoconjugate therapeutic of an embodiment of the present application to the selected subject.

As used herein, the term “treat” refers to the application or administration of the immunoconjugate therapeutic of the present application to a subject, e.g., a patient. The treatment can be to cure, heal, alleviate, relieve, alter, remedy, ameliorate, palliate, improve or affect the cancer, the symptoms of the cancer or the predisposition toward the cancer.

As used herein, the term “subject” is intended to include human and non-human animals. Non-human animals include all vertebrates, e.g., mammals and non-mammals, such as non-human primates, sheep, dog, cow, chickens, amphibians, reptiles, etc.

In one embodiment, the cancer is selected from small cell and non-small cell lung cancer, rhabdomyosarcoma, breast cancer, pancreatic cancer, Wilm's tumor, multiple myeloma, neuroblastoma, and glioma.

In one embodiment, the subject is a mammalian subject. In another embodiment, the subject is a human subject.

Another aspect of the present application is a method of targeted intracellular delivery of an anti-cancer therapeutic to a target cell population, said method comprising selecting a population of target cells, wherein the population of target cells is positive for polysialic acid (polySia) and administering an immunoconjugate therapeutic of the present application to the selected target cell population.

In one embodiment, administering an immunoconjugate of the present application to a target cell population is carried out in vitro. In another embodiment, administering an immunoconjugate of the present application to a targeted cell population is carried out ex vivo. In a further embodiment, administering an immunoconjugate of the present application to a targeted cell population is carried out in vivo.

In one embodiment, the target cell population is a population of mammalian cells. In an further embodiment, the target cell population is a population of human cells.

The immunoconjugate therapeutics of the present application can be administered in combination with other therapeutics and/or adjuvants. In one embodiment, the methods of the present application comprise administering, for example, chemotherapeutic agents, epigenetic agents, or ionizing radiation. In a further embodiment, the method of treating subjects with cancer further comprises administering to the subject a chemotherapeutic agent, an epigenetic agent, or ionizing radiation.

As used herein, the terms “maximum tolerated dose (MTD)” refers to the dose of any therapeutic drug—including targeted drugs—above which unacceptable toxicity occurs. This is true whether the drugs are targeted to a particular cell type or a particular molecule. Because of the MTD and the limit of tolerability of a drug (targeted or otherwise), maximal anti-cancer efficacy is generally not attainable. The MTD of a drug is impacted significantly by its biodistribution and its pharmacokinetics.

As used herein, the term “biodistribution” refers to the organs and tissues to which a drug distributes in the body.

As used herein, the term “pharmacokinetics” refers to how long a drug stays in the body.

In one embodiment, the immunoconjugate therapeutic is administered in an amount effective to treat a subject.

In one embodiment, the immunoconjugate therapeutic is administered as part of a composition.

Effective doses of the immunoconjugate therapeutics and compositions of the present application, for the treatment of cancer or the targeted intracellular delivery of an anti-cancer therapeutic vary depending upon many different factors, including the physiological environment in which the immunoconjugate therapeutic is administered (e.g. in vitro, ex vivo, in vivo), type and stage of cancer, means of administration, target site, physiological state of the patient, other medications or therapies administered, and physical state of the patient relative to other medical complications. Treatment dosages need to be titrated to optimize safety and efficacy.

It will be appreciated that when treating a subject, the exact dosage of the immunoconjugate therapeutics of the present application is chosen by the individual physician in view of the subject to be treated. In general, dosage and administration are adjusted to provide an effective amount of the immunoconjugate therapeutic to the subject being treated. As used herein, the “effective amount” of an immunoconjugate therapeutic refers to the amount necessary to elicit the desired biological response. As will be appreciated by those of ordinary skill in this art, the effective amount of immunoconjugate therapeutic of the present application may vary depending on such factors as the desired biological endpoint, the drug to be delivered, the target, the route of administration, etc. For example, the effective amount of immunoconjugate therapeutic comprising an anti-cancer therapeutic might be the amount that results in a reduction in tumor size by a desired amount over a desired period of time. Additional factors which may be taken into account include the severity of the disease state; age, weight and gender of the patient being treated; diet, time and frequency of administration; drug combinations; reaction sensitivities; and tolerance/response to therapy.

In general, doses can range from about 25% to about 100% of the MTD of the immunoconjugate therapeutic. Based upon the composition of the immunoconjugate therapeutic, the dose can be delivered once, continuously, such as by continuous pump, or at periodic intervals. Dosage may be adjusted appropriately to achieve desired drug levels, locally, or systemically. In the event that the response in a subject is insufficient at such doses, even higher doses (or effective higher doses by a different, more localized delivery route) may be employed to the extent that patient tolerance permits. Continuous IV dosing over, for example, 24 hours or multiple doses per day also are contemplated to achieve appropriate systemic levels of compounds

In one embodiment of the method of the present application, the administering of the immunoconjugate therapeutic results in cytotoxicity of the targeted cell(s). In another embodiment, administering of the immunoconjugate therapeutic results in increased percent cytotoxicity of the targeted cells compared to percent cytotoxicity of untreated targeted cells. In a further embodiment, administering of the immunoconjugate therapeutic results in increased cytotoxicity of the targeted cells compared to percent toxicity of target cells treated with a control treatment. In an additional embodiment, percent viability of the targeted cell(s) is in a range from about 0% to 70%, 0% to 60%, 0% to 50%, 0% to 40%, 0% to 30%, 0% to 20%, 0% to 10%, 0% to 5%, 10% to 70%, 10% to 60%, 10% to 50%, 10% to 40%, 10% to 30%, 10% to 20%, 20% to 70%, 20% to 60%, 20% to 50%, 20% to 40%, 20% to 30%, 30% to 70%, 30% to 60%, 30% to 50%, 30% to 40%, 40% to 70%, 40% to 60%, 40% to 50%, 50% to 70%, 50% to 60%, or 60% to 70%.

In one embodiment, the immunoconjugate therapeutic is administered at a concentration (in nM) of about 0.1 to 100, 0.1 to 90, 0.1 to 80, 0.1 to 70, 0.1 to 60, 0.1 to 50, 0.1 to 40, 0.1 to 30, 0.1 to 20, 0.1 to 10, 0.1 to 1, 1 to 100, 1 to 90, 1 to 80, 1 to 80, 1 to 70, 1 to 60, 1 to 50, 1 to 40, 1 to 30, 1 to 20, 1 to 10, 10 to 100, 10 to 90, 10 to 80, 10 to 70, 10 to 60, 10 to 50, 10 to 40, 10 to 30, 10 to 20, 20 to 100, 20 to 90, 20 to 80, 20 to 70, 20 to 60, 20 to 50, 20 to 40, 20 to 30, 30 to 100, 30 to 90, 30 to 80, 30 to 70, 40 to 60, 40 to 50, 50 to 100, 50 to 90, 50 to 80, 50 to 70, 50 to 60, 60 to 100, 60 to 90, 60 to 80, 60 to 70, 70 to 100, 70 to 90, 70 to 80, 80 to 100, 80 to 90, or 90 to 100.

In methods of the present application, the administering step is carried out to treat cancer in a subject. In one embodiment, a subject having cancer characterized by polysialic acid (polySia)-positive tumor cells is selected prior to the administering step. Such administration can be carried out systemically or via direct or local administration to the tumor site. By way of example, suitable modes of systemic administration include, without limitation orally, topically, transdermally, parenterally, intradermally, intramuscularly, intraperitoneally, intravenously, subcutaneously, or by intranasal instillation, by intracavitary or intravesical instillation, intraocularly, intraarterialy, intralesionally, or by application to mucous membranes. Suitable modes of local administration include, without limitation, catheterization, implantation, direct injection, dermal/transdermal application, or portal vein administration to relevant tissues, or by any other local administration technique, method or procedure generally known in the art. The mode of affecting delivery of immunoconjugate therapeutic will vary depending on the type of therapeutic agent (e.g., an antibody or an inhibitory nucleic acid molecule) and the disease to be treated.

The immunoconjugate therapeutics of the present application may be orally administered, for example, with an inert diluent, or with an assimilable edible carrier, or it may be enclosed in hard or soft shell capsules, or it may be compressed into tablets, or they may be incorporated directly with the food of the diet. Immunoconjugate therapeutics of the present application may also be administered in a time release manner incorporated within such devices as time-release capsules or nanotubes. Such devices afford flexibility relative to time and dosage. For oral therapeutic administration, the immunoconjugate therapeutics of the present application may be incorporated with excipients and used in the form of tablets, capsules, elixirs, suspensions, syrups, and the like. Such compositions and preparations should contain at least 0.1% of the immunoconjugate therapeutic, although lower concentrations may be effective and indeed optimal. The percentage of the immunoconjugate therapeutic in these compositions may, of course, be varied and may conveniently be between about 2% to about 60% of the weight of the unit. The amount of an immunoconjugate therapeutic of the present application in such therapeutically useful compositions is such that a suitable dosage will be obtained.

When the immunoconjugate therapeutics of the present application are administered parenterally, solutions or suspensions of the agent can be prepared in water suitably mixed with a surfactant such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions. Under ordinary conditions of storage and use, these preparations contain a preservative to prevent the growth of microorganisms.

Pharmaceutical formulations suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

When it is desirable to deliver the immunoconjugate therapeutics of the present application systemically, they may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Intraperitoneal or intrathecal administration of the immunoconjugate therapeutics of the present application can also be achieved using infusion pump devices. Such devices allow continuous infusion of desired compounds avoiding multiple injections and multiple manipulations.

In addition to the formulations described previously, the immunoconjugate therapeutics may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

Another aspect of the present application relates to a pharmaceutical composition comprising an immunoconjugate therapeutic of the present application. In an embodiment, the composition further comprises a carrier. That pharmaceutical composition can be formulated as described above.

### EXAMPLES

The examples below are intended to exemplify the practice of embodiments of the disclosure but are by no means intended to limit the scope thereof.

### Example 1—Introduction

To investigate polySia targeting and its clinical potential, the mo735 and ch735 antibodies were subjected to a spectrum of biochemical and cell biological assays to characterize their polySia binding properties. Importantly, both antibodies were observed to bind polySia with high affinity and exquisite selectivity. It was also confirmed that both antibodies recognized several polySia-positive tumor cell lines in vitro and induced rapid internalization of polySia into endosomal and lysosomal compartments. In light of these findings, it was hypothesized that the antibody-induced endocytosis of polySia-receptors could be efficiently harnessed as part of an antitumor therapeutic strategy. To test this notion, an ADC was engineered using a bioorthogonal reaction scheme for stably linking the chimeric human ch735 mAb to the microtubule-inhibitory agent maytansinoid DM1, which has previously been developed as the cytotoxic payload in trastuzumab emtansine (T-DM1) for HER2-positive breast cancer (Verma et al., “Trastuzumab Emtansine for HER2-Positive Advanced Breast Cancer,” N. Engl. J. Med. 367:1783-91 (2012), which is hereby incorporated by reference in its entirety). The resulting conjugate was found to exert potent target-dependent cytotoxicity against polySia-positive tumor cells in vitro, providing compelling proof-of-concept for the use of polySia-receptor internalization as a carrier for delivery of cytotoxic payloads to cancer cells. Taken together, these findings add to the growing body of literature implicating aberrant glycans in the tumor glycocalyx as an attractive collection of targets for the development of glycan-directed synthetic immunotherapies.

### Example 2—Construction and Characterization of a Chimeric Human IgG Targeting polySia

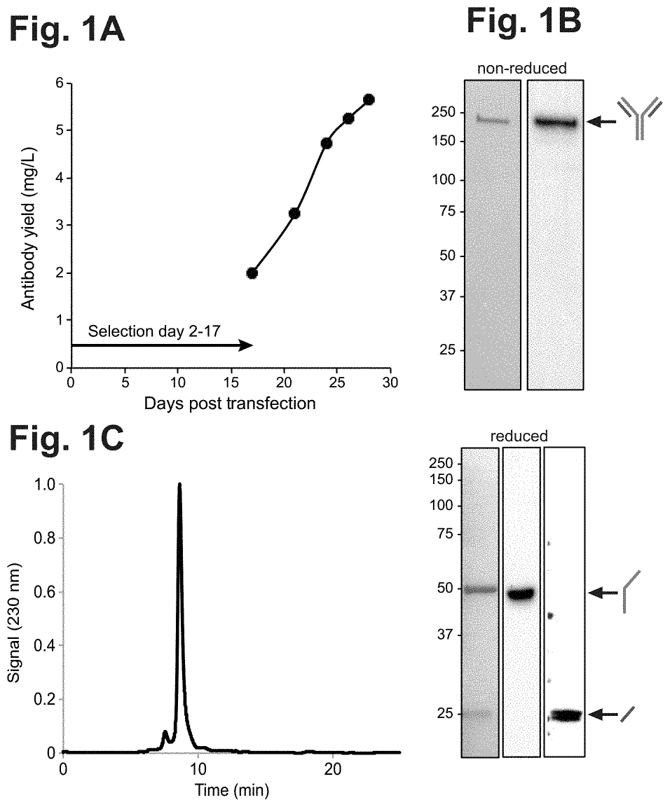

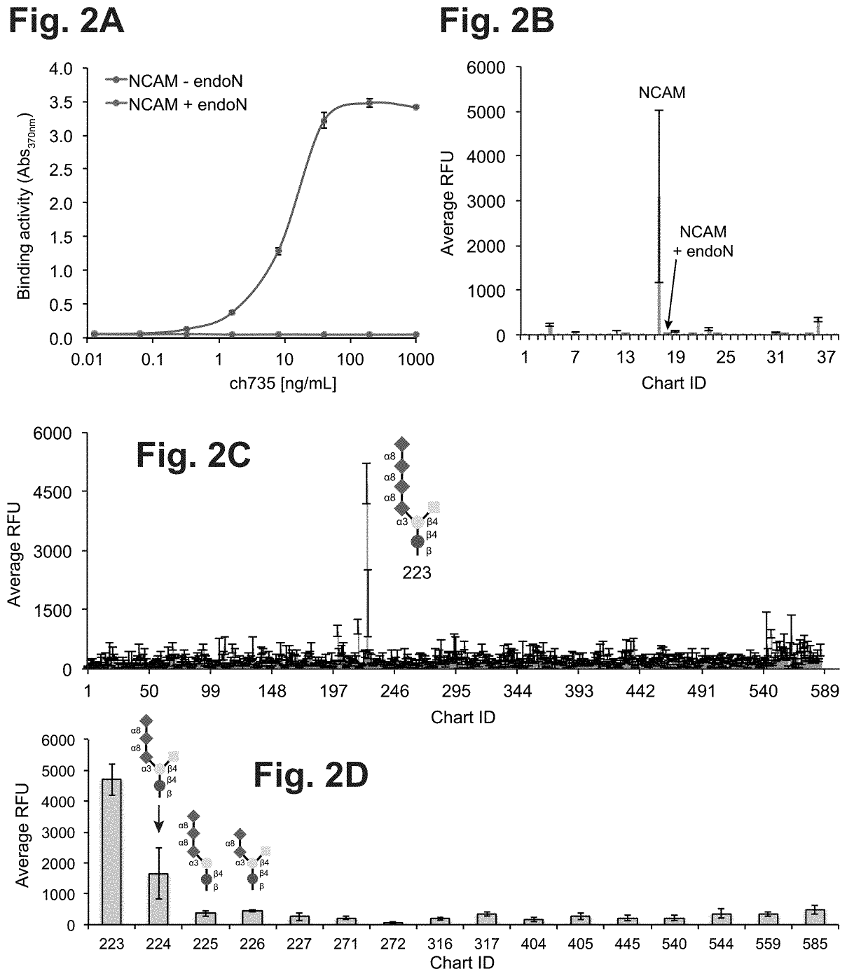

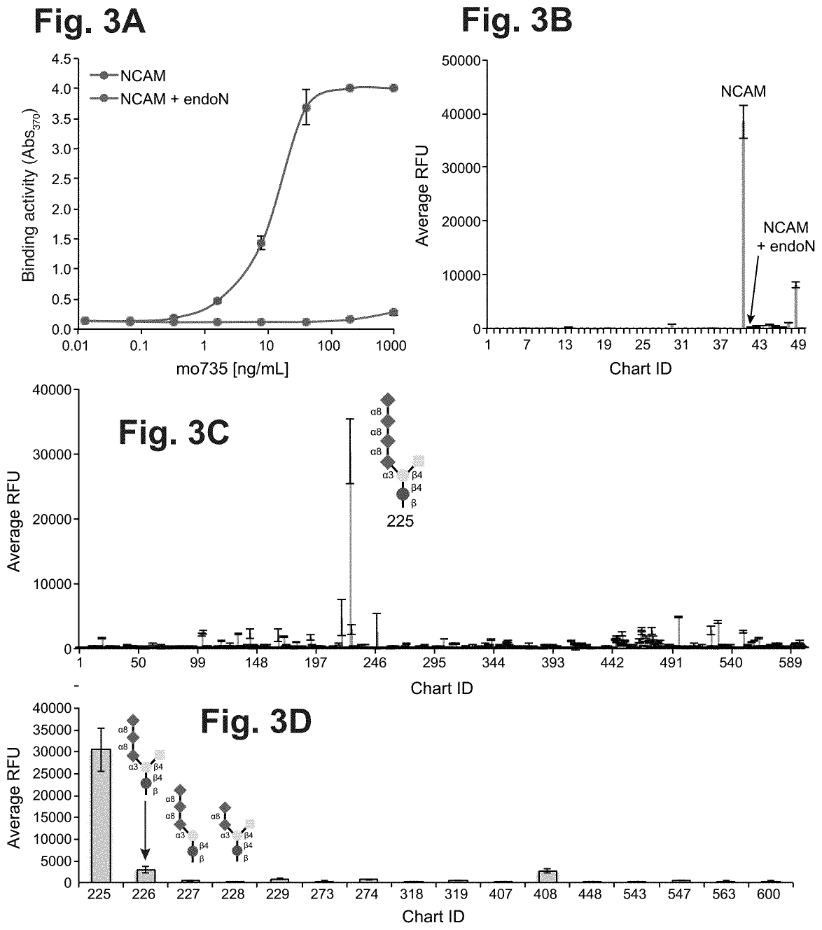

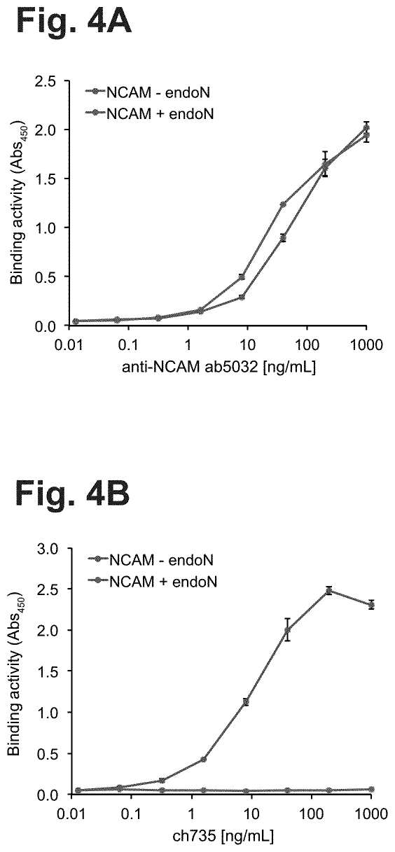

To generate a monoclonal antibody (mAb) that is more compatible with targeting human cancers, the fully mouse IgG2a mAb 735 (mo735) was converted into a chimeric human IgG1 (ch735) by swapping the variable regions according to an antibody cloning and expression method described by Beavil and coworkers (Dodev et al., “A Tool Kit for Rapid Cloning and Expression of Recombinant Antibodies,” Sci. Rep. 4:5885 (2014), which is hereby incorporated by reference in its entirety). Using this approach, a stable cell line was generated that was capable of producing fully assembled ch735, which could be purified to near homogeneity at yields up to 6 mg/L (FIGS. 1A-1C). Subsequent enzyme-linked immunosorbent assay (ELISA) analysis confirmed that both antibodies bound chicken brain-derived polysialylated neural cell adhesion molecule (NCAM) but not NCAM that was treated with endoneuraminidase N (endoN) that selectively removes polySia (FIGS. 2A-2D; FIG. 3A). Probing of similarly prepared ELISA plates with an NCAM-specific antibody confirmed that both NCAM and endoN-treated NCAM were equally coated on ELISA plates (FIGS. 4A-4B). Given the strict specificity of endoN for α2,8-linkages in sources as disparate as bacterial and neural membrane glycoconjugates, we conclude that both mo735 and ch735 specifically recognize polySia.

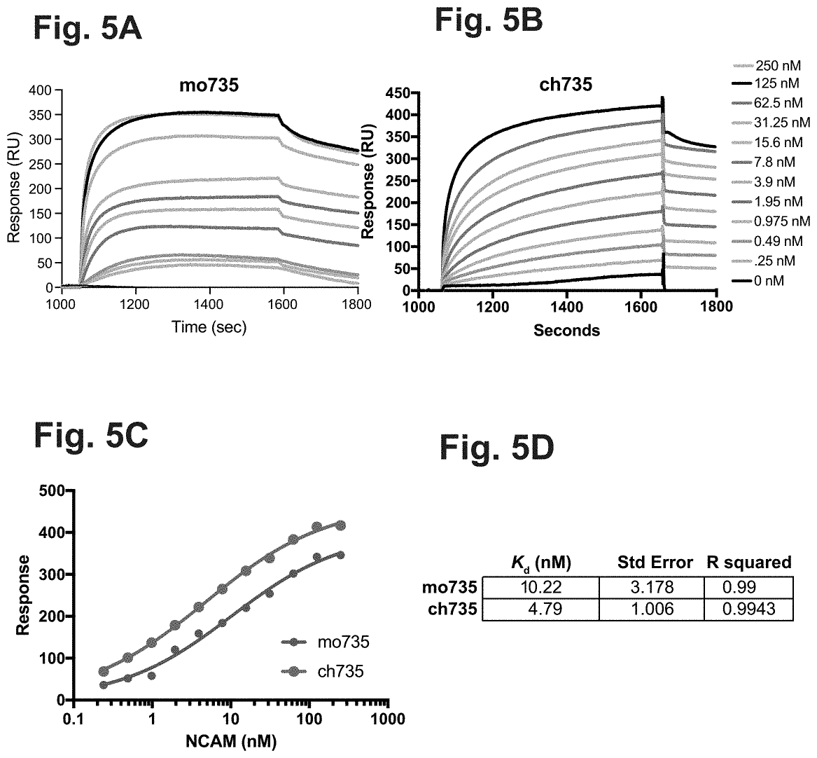

To investigate glycan specificity, both antibodies were also analyzed on a glycoprotein array that contained ˜50 glycoproteins and the current glycan array (version 5.3 for mo735, version 5.4 for ch735) of the Consortium for Functional Glycomics (CFG) that contained ˜600 natural and synthetic mammalian glycans (http://www.functionalglycomics.org). The chimeric human IgG1 ch735 showed a strong preference for polysialylated NCAM in the glycoprotein array (FIG. 2A, Chart ID #17) and the tetra-sialic acid containing glycan GalNAcβ1-4(Neu5Acα2-8Neu5Acα2-8Neu5Acα2-8Neu5Acα2-3)Galβ1-4Glcβ-Sp0 in the glycan array (FIGS. 2C-2D, Chart ID #223 in microarray version 5.4). A lesser but still significant level of binding above background was detected for GalNAcβ1-4(Neu5Acα2-8Neu5Acα2-8Neu5Acα2-3)Galβ1-4Glcβ-Sp0 (FIG. 2D, Chart ID #224), but not for the closely related glycans #225 and 226 that differed from 224 by a single branched GalNac and Neu5Ac, respectively (FIG. 2D). In total, the glycan microarray included 145 glycans containing some form of sialic acid, often as the terminal sugar, of which 16 were α2,8-linked (Table 1); hence, it was conclude unequivocally that the specificity of ch735 is for α2,8-linked sialic acid with a degree of polymerization (DP) of three or greater. Importantly, there was no significant signal towards any other glycomolecules including endoN-treated NCAM that was spotted on the glycoprotein array. Nearly identical results were observed when the microarrays were probed with mo735 (FIGS. 3B-3D). To determine affinity, the equilibrium binding of both antibodies to polysialylated NCAM was measured using surface plasmon resonance (SPR). Binding values were fit using the specific binding with Hill slope analysis in Prism software and the calculated K_{d} values for mo735 and ch735 were determined to be 10.22 and 4.79 nM, respectively (FIGS. 5A-5D). These values were in close agreement with the previously reported K_{d} of ˜5 nM for the mouse IgG against embryonic brain glycopeptides (Hayrinen et al., “High Affinity Binding of Long-Chain Polysialic Acid to Antibody, and Modulation by Divalent Cations and Polyamines,” Mol. Immunol. 39:399-411 (2002), which is hereby incorporated by reference in its entirety).

TABLE 1
CFG microarray glycans containing α2,8-linked sialic acid

| CFG ID | |
|---|---|
| (v5.3/v5.4)¹ | Glycan structure |
| 225/223² | GalNAcβ1-4(Neu5Acα2-8Neu5Acα2-8Neu5Acα2- |
| | 8Neu5Acα2-3)Galβ1-4Glcβ-Sp0 |
| 226/224 | GalNAcβ1-4(Neu5Acα2-8Neu5Acα2-8Neu5Acα2- |
| | 3)Galβ1-4Glcβ-Sp0 |
| 227/225 | Neu5Acα2-8Neu5Acα2-8Neu5Acα2-3Galβ1-4Glcβ-Sp0 |
| 228/226 | GalNAcβ1-4(Neu5Acα2-8Neu5Acα2-3)Galβ1-4Glcβ-Sp0 |
| 229/227 | Neu5Acα2-8Neu5Acα2-8Neu5Acα-Sp8 |
| 273/271 | Neu5Acα2-8Neu5Acα-Sp8 |
| 274/272 | Neu5Acα2-8Neu5Acα2-3Galβ1-4Glcβ-Sp0 |
| 318/316 | Neu5Acα2-8Neu5Acβ-Sp17 |
| 319/317 | Neu5Acα2-8Neu5Acα2-8Neu5Acβ-Sp8 |
| 407/404 | Galβ1-3GalNAcβ1-4(Neu5Acα2-8Neu5Acα2-3)Galβ1- |
| | 4Glcβ-Sp0 |
| 408/405 | Neu5Acα2-3Gaβ1-3GalNAcβ1-4(Neu5Acα2-8Neu5Acα2- |
| | 3)Galβ1-4Glcβ-Sp0 |
| 448/445 | Neu5Acα2-8Neu5Acα2-3Galβ1-3GalNAcβ1-4(Neu5Acα2- |
| | 8Neu5Acα2-3)Galβ1-4Glcβ-Sp0 |
| 543/540 | Neu5Acα2-8Neu5Gcα2-3Galβ1-4GlcNAc-Sp0 |
| 547/544 | Neu5Acα2-8Neu5Acα2-3Galβ1-4GlcNAc-Sp0 |
| 563/559 | Neu5Acα2-8Neu5Acα2-3Galβ1-3GalNAcβ1-4(Neu5Acα2- |
| | 3)Galβ1-4Glc-Sp21 |
| 600/585 | Galβ1-3GalNAcβ1-4(Neu5Acα2-8Neu5Acα2-8Neu5Acα2- |
| | 3)Galβ1-4Glcβ-Sp21 |

¹ID numbers correspond to CFG glycan microarray version 5.3 (used for mo735) and version 5.4 (used for ch735);
²Glycan 225/223 gave the strongest binding signal in FIGS. 2A-2D and 3A-3D

### Example 3—Antibody Ch735 Binds Surface polySia and is Internalized in Cancer Cells

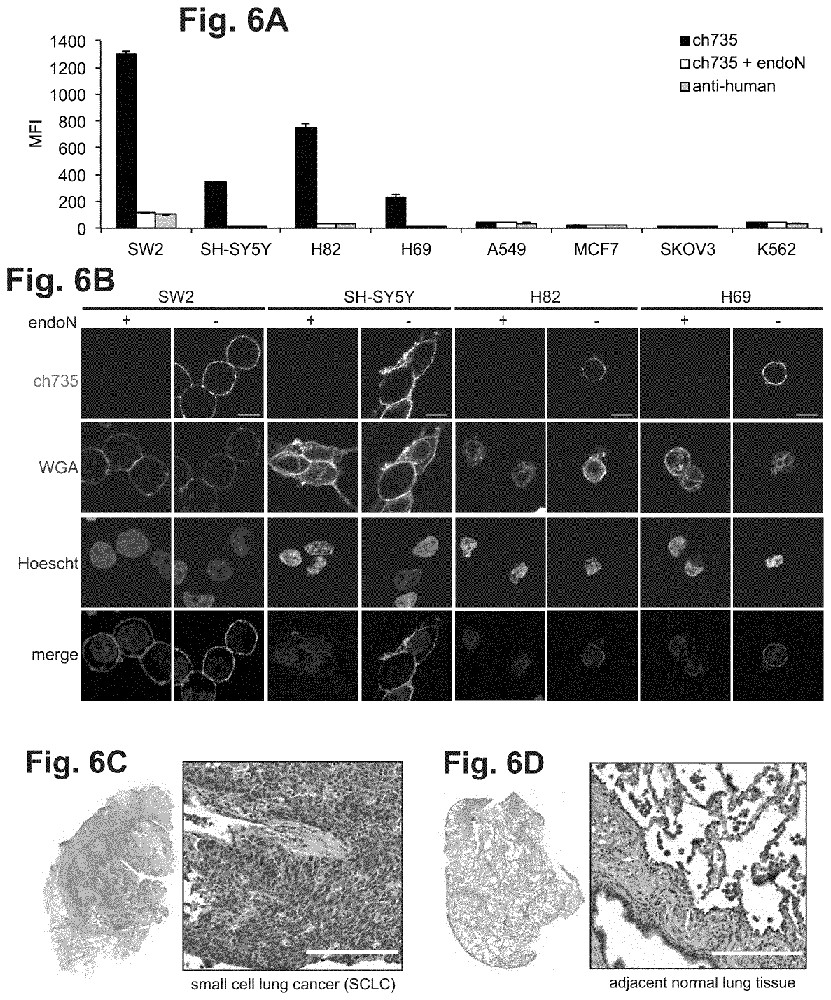

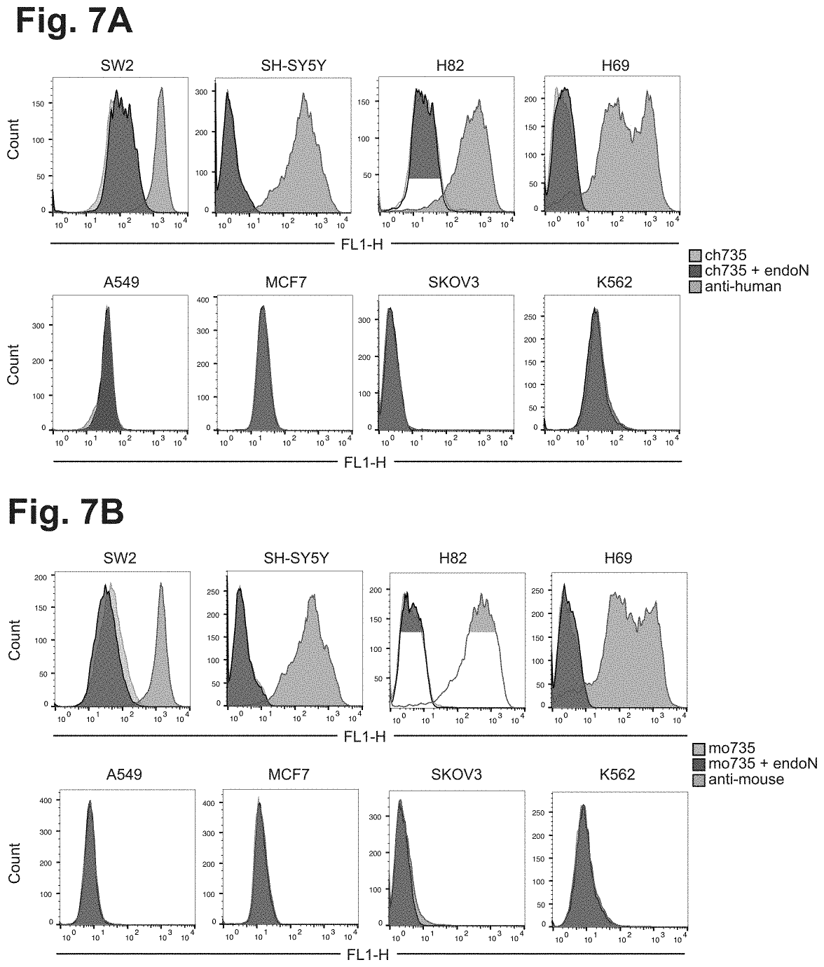

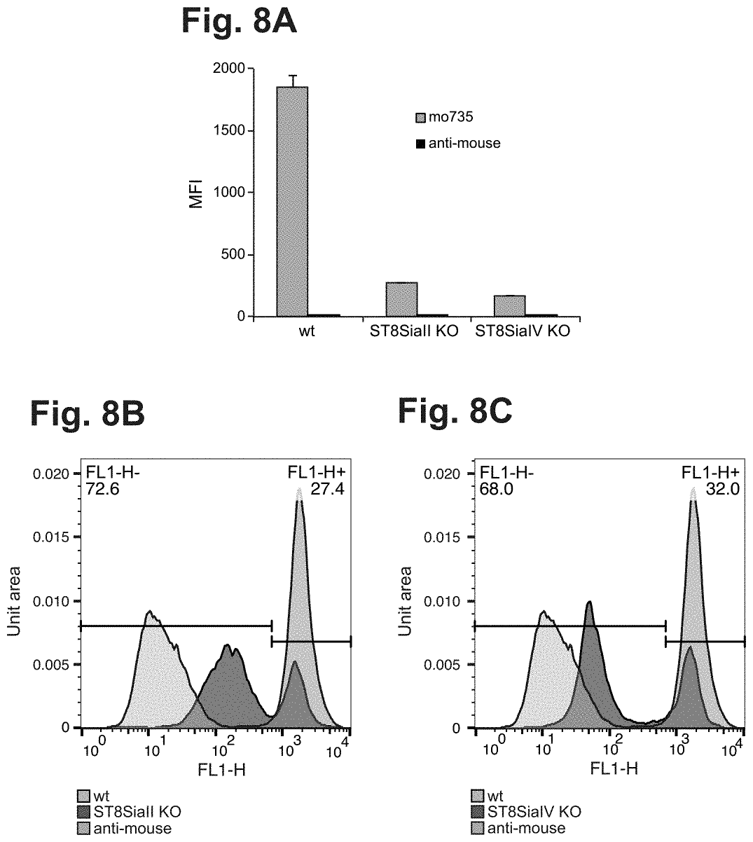

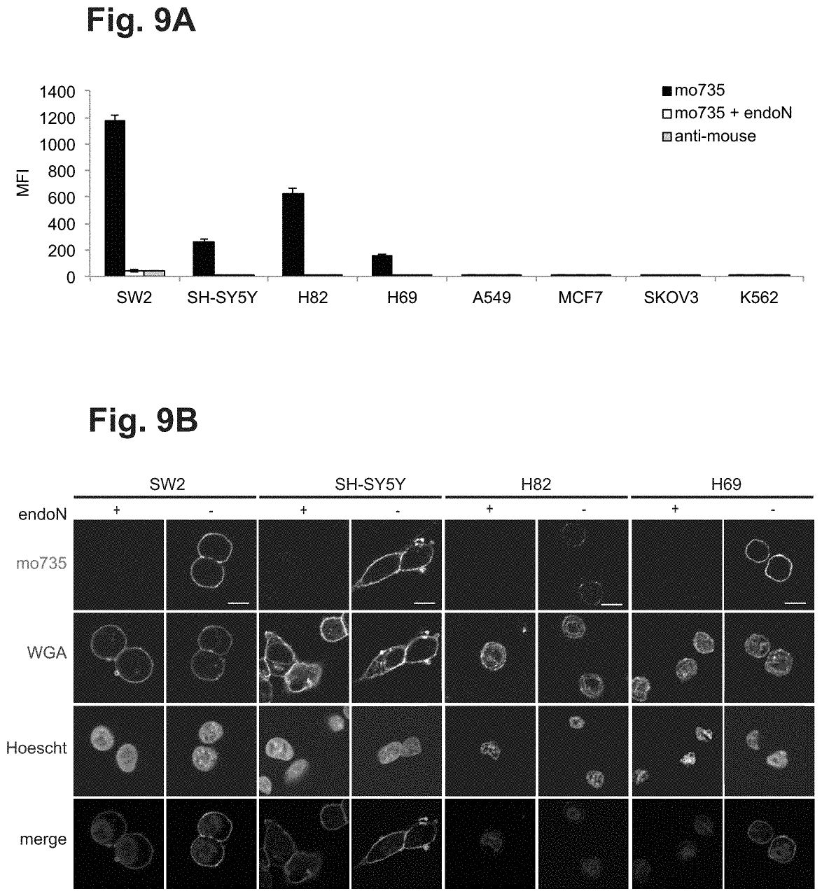

To demonstrate the relevance of this antibody in the context of human cancers, flow cytometric analysis was used to assess ch735 binding to polySia expressed on the surface of different cancer cell lines including the small cell lung cancer (SCLC) cell lines SW2, NCI-H69, and NCI-H82, as well as neuroblastoma cell line SH-SY5Y, non-small cell lung cancer (NSCLC) cell line A549, breast cancer cell line MCF7, ovarian cancer cell line SKOV3, and chronic myeloid leukemia (CML) cell line K562. Antibody ch735 bound most avidly to SW2 cells, and also recognized NCI-H69, NCI-H82, and SH-SY5Y cancer cells although with a lower intensity (FIG. 6A), in agreement with previous cell line characterization studies (Martersteck et al., “Unique Alpha 2,8-Polysialylated Glycoproteins in Breast Cancer and Leukemia Cells,” Glycobiology 6:289-301 (1996); Zapater et al., “Sequences Prior to Conserved Catalytic Motifs of Polysialyltransferase ST8Sia IV are Required for Substrate Recognition,” J. Biol. Chem. 287:6441-53 (2012); Valentiner et al., “Expression of the Neural Cell Adhesion Molecule and Polysialic Acid in Human Neuroblastoma Cell Lines,” Int. J. Oncol. 39:417-24 (2011); and Livingston et al., “Selection of GM2, Fucosyl GM1, Globo H and Polysialic Acid as Targets on Small Cell Lung Cancers for Antibody Mediated Immunotherapy,” Cancer Immuno.l Immunother. 54:1018-25 (2005), which are hereby incorporated by reference in their entirety). The representative histograms revealed not only differing levels of surface expression between the different polySia-positive cancer cells but also within each population especially for H69 cells (FIG. 7A). The lack of surface binding following endoN treatment confirmed that binding to these cell lines was specific to polySia. Further evidence of polySia-specific binding was demonstrated by a significant decrease in antibody labeling of SW2 cells in which the polysialyltransferases ST8SiaII and ST8SiaIV were knocked out by CRISPR/Cas9 gene editing (FIGS. 8A-8C). No significant binding above background was observed for A549 and MCF7 cancer cells, in agreement with previous studies (Hromatka et al., “Polysialic Acid Enhances the Migration and Invasion of Human Cytotrophoblasts,” Glycobiology 23:593-602 (2013) and Martersteck et al., “Unique Alpha 2, 8-Polysialylated Glycoproteins in Breast Cancer and Leukemia Cells,” Glycobiology 6:289-301 (1996), which are hereby incorporated by reference in their entirety). Likewise, SKOV3 and K562 cells were not recognized by ch735, confirming these as polySia-negative cell lines. The ability of ch735 to recognize polySia on the cell surface was corroborated by immunofluorescence microscopic images of the polySia-positive cell lines (FIG. 6B). Identical polySia binding results were obtained following staining of each of these cell lines with mo735 (FIGS. 7B and 9A-9B). In addition, immunohistochemistry (IHC) revealed strong staining of polySia in formalin-fixed, paraffin-embedded (FFPE) human tissue sections of SCLC (FIG. 6C), but little to no staining of the adjacent normal tissue except for the bronchial epithelial cells and alveolar macrophages (FIG. 6D), in close agreement with previous findings (Zhang et al., “Selection of Tumor Antigens as Targets for Immune Attack Using Immunohistochemistry: I. Focus on Gangliosides,” Int. J. Cancer 73:42-9 (1997), which is hereby incorporated by reference in its entirety).

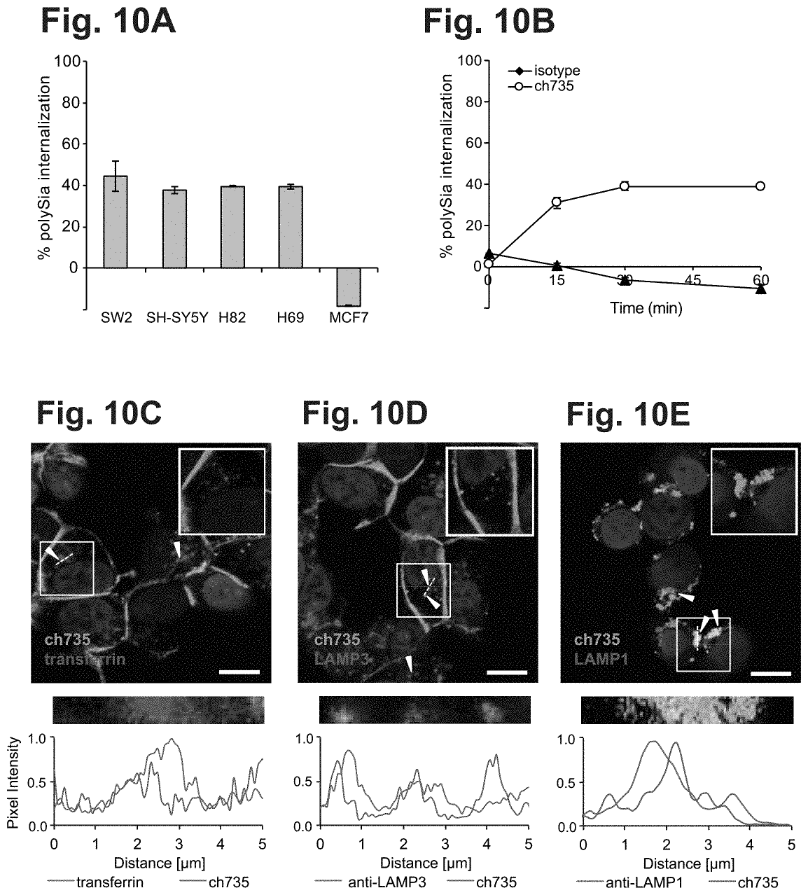

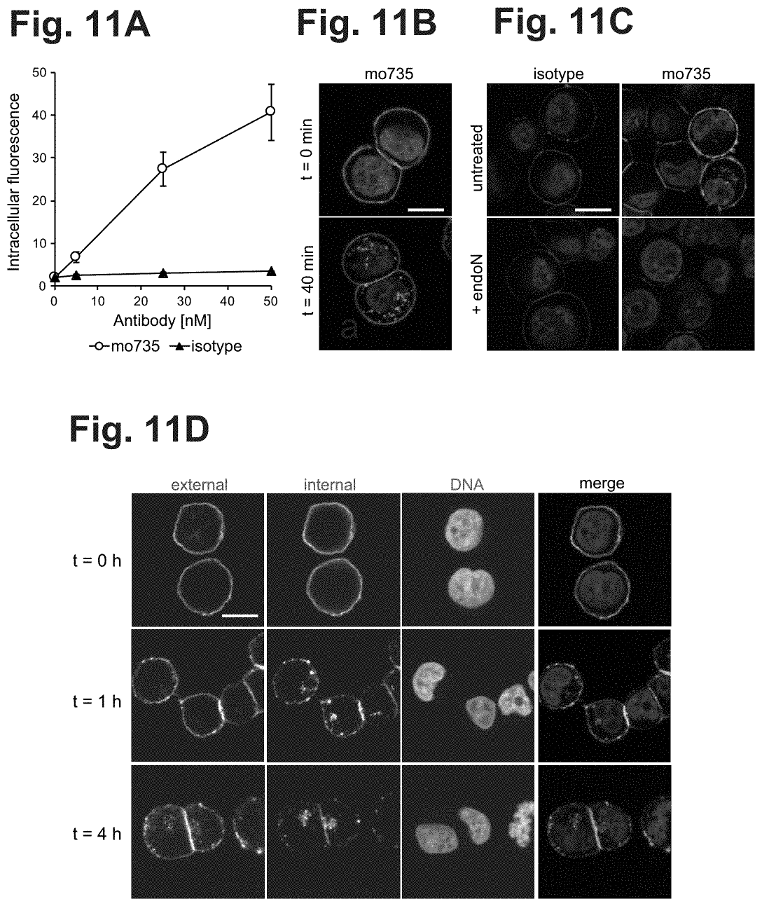

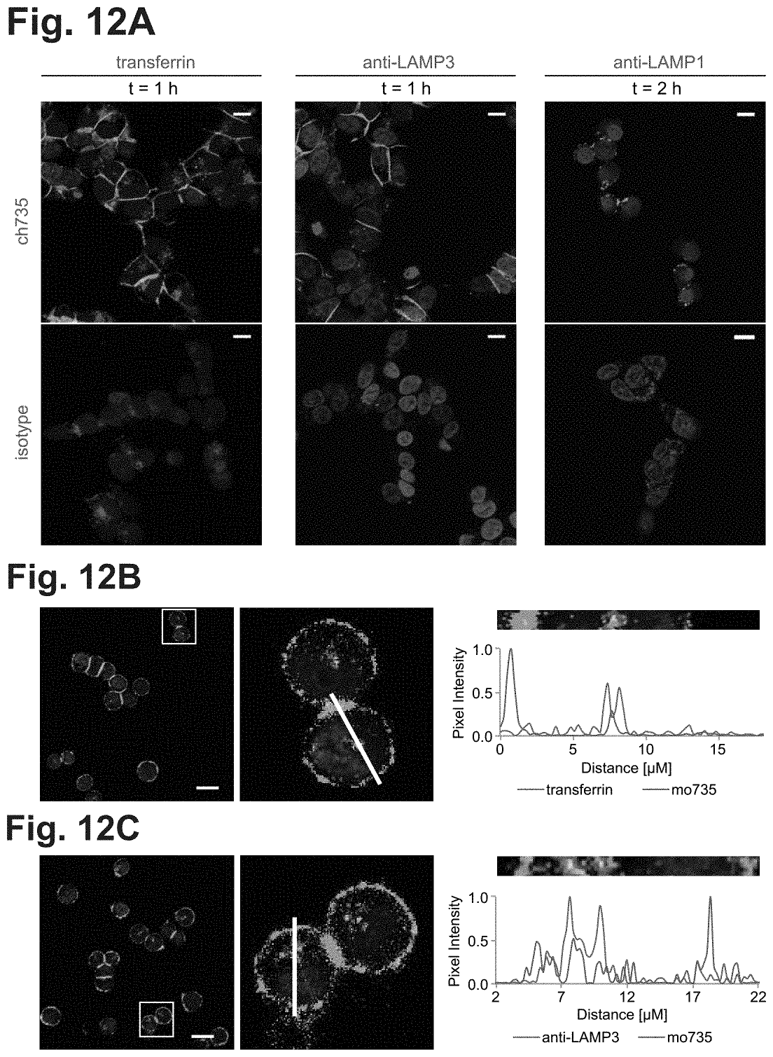

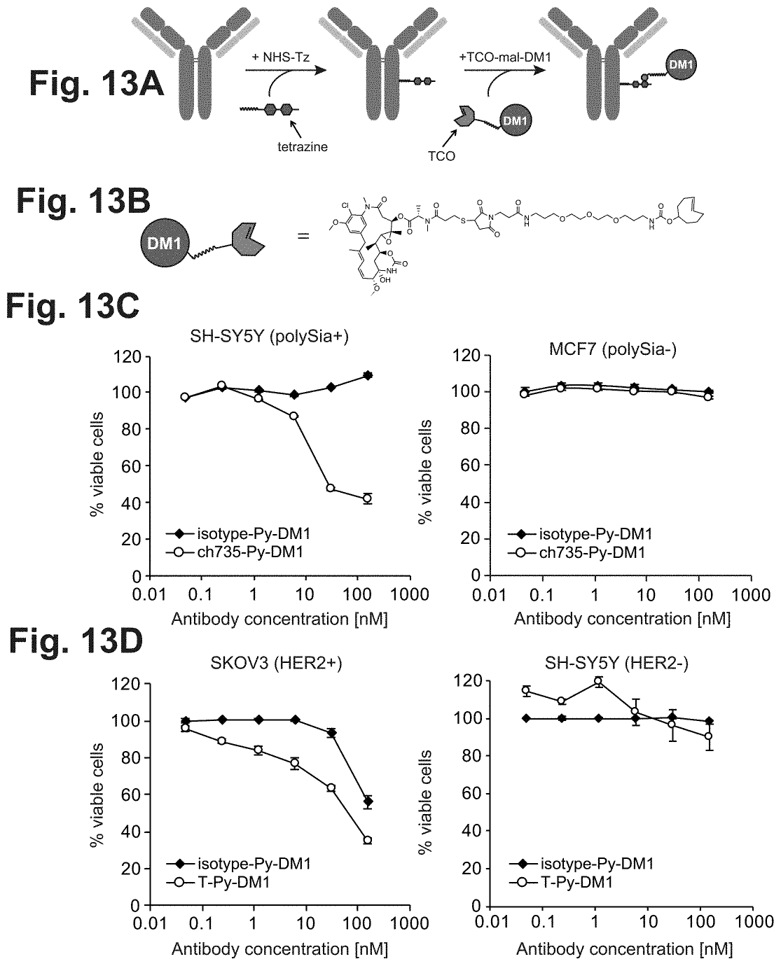

Given that clathrin-mediated endocytosis is an essential pathway by which many glycoproteins are recycled or down-regulated (Goldstein et al., “Receptor-Mediated Endocytosis: Concepts Emerging from the LDL Receptor System,” Annu. Rev. Cell Biol. 1:1-39 (1985), which is hereby incorporated by reference in its entirety), it was next investigated whether polySia undergoes a similar internalization process. Previous studies demonstrated that NCAM, one of the major carriers of polySia, was recycled by a clathrin-dependent endocytosis process (Diestel et al., “NCAM is Ubiquitylated, Endocytosed and Recycled in Neurons,” J. Cell Sci. 120:4035-49 (2007) and Minana et al., “Neural Cell Adhesion Molecule is Endocytosed Via a Clathrin-Dependent Pathway,” Eur. J. Neurosci. 13:749-56 (2001), which are hereby incorporated by reference in their entirety), whereas polySia was only detectable at the cell surface (Martersteck et al., “Unique Alpha 2, 8-Polysialylated Glycoproteins in Breast Cancer and Leukemia Cells,” Glycobiology 6:289-301 (1996) and Zuber et al., “The Relationship of Polysialic Acid and the Neural Cell Adhesion Molecule N-CAM in Wilms Tumor and their Subcellular Distributions,” Eur. J. Cell Biol. 51:313-21 (1990), which are hereby incorporated by reference in their entirety) unless internalization was activated by the extracellular matrix (ECM) (Monzo et al., “Insulin and IGF1 Modulate Turnover of Polysialylated Neural Cell Adhesion Molecule (PSA-NCAM) in a Process Involving Specific Extracellular Matrix Components,” J. Neurochem 126:758-70 (2013), which is hereby incorporated by reference in its entirety). To further investigate this issue here, each cell line that expressed cell surface polySia was evaluated for the ability to internalize polySia. This involved first binding ch735 to the surface of tumor cells at 4° C., after which an aliquot of cells remained at 4° C. while the rest were incubated at 37° C. and analyzed by flow cytometry at different time points. For each of the polySia-positive cell lines, we observed that ˜40% of the antibody was internalized after 1 h while no internalization was observed for the MCF7 cell line (FIG. 10A), which were previously found to lack polySia at the cell surface (Martersteck et al., “Unique Alpha 2, 8-Polysialylated Glycoproteins in Breast Cancer and Leukemia Cells,” Glycobiology 6:289-301 (1996), which is hereby incorporated by reference in its entirety). A time course of ch735 binding to SH-SY5Y cells revealed that antibody internalization occurred rapidly, with ˜30% of the antibody internalized as early as 15 min and maximum internalization of 40% reached by 30 min (FIG. 10B). In contrast, an isotype control antibody showed no measurable internalization over the same time period. It is noteworthy that the internalization percentage and rate observed here with ch735 was on par with that reported previously with trastuzumab against HER-2-positive cancer cells (Li et al., “A Biparatopic HER2-Targeting Antibody-Drug Conjugate Induces Tumor Regression in Primary Models Refractory to or Ineligible for HER2-Targeted Therapy,” Cancer Cell 29:117-29 (2016), which is hereby incorporated by reference in its entirety). It should also be noted that comparable internalization of mAb mo735 into SW2 cells was observed, with intracellular fluorescence increasing as a function of polySia-specific antibody concentration and as a function of time (FIGS. 11A-11D).

Confocal microscopy was used to investigate the compartments where the ch735 mAb accumulated after internalization using markers of early endosomes, recycling endosomes or late endosome/lysosomes. Consistent with flow cytometry, ch735 initially labeled the plasma membrane of SH-SY5Y cells and after 1 h at 37° C. was internalized, where it clearly colocalized with early endosomal and recycling endosomal marker transferrin (FIG. 10C) and late endosomal marker LAMP-3 (FIG. 10D). Accumulation of the ch735 mAb was also observed in late endosomal/lysosomal LAMP-1-positive compartments (FIG. 10E). As expected, no detectable binding, internalization or colocalization was observed for the isotype control (FIG. 12A). Similar to ch735, the mo735 mAb compartmentalized in early and recycling endosomes as confirmed by colocalization with transferrin and LAMP-3 (FIGS. 12B-12C). Based on these data, it was concluded that mAb ch735 binds to tumor cell membranes in a target-specific manner, thereby inducing a subpopulation of bound antibodies to become rapidly internalized in endosomal/lysosomal compartments.

### Example 4—Glycan-Directed ADC is Cytotoxic Against Tumor Cells Expressing polySia

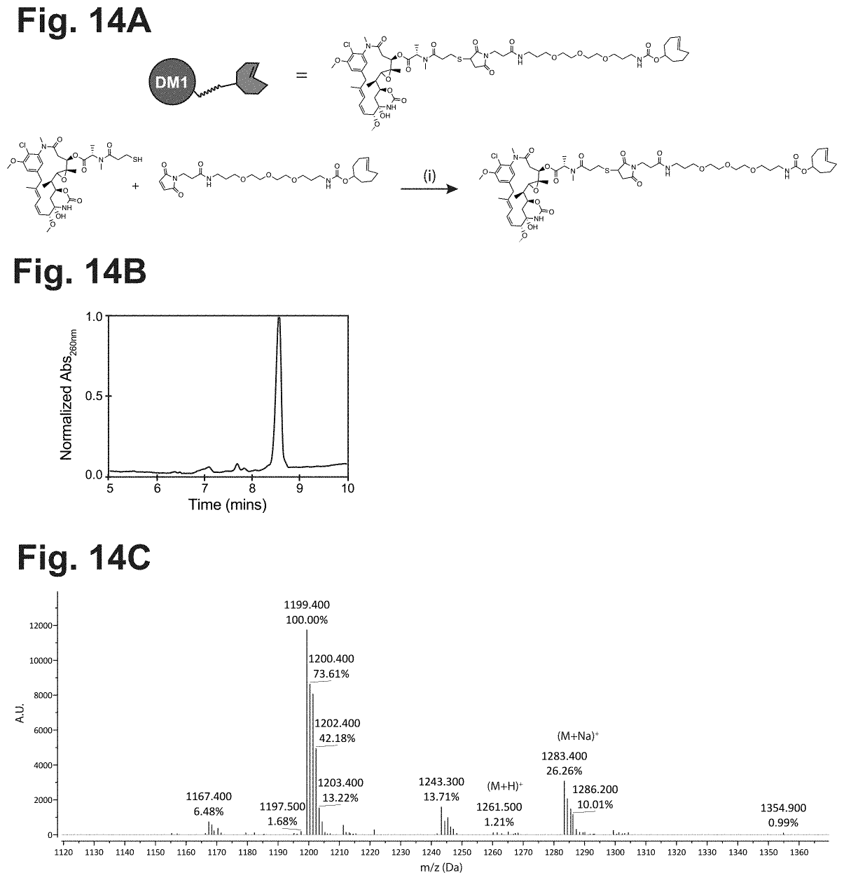

Given that ch735 induced internalization of polySia receptors in cancer cells, it was next evaluated whether drug conjugation could be used to confer target-specific in vitro cytotoxicity to mAb ch735. To this end, a covalent, bioorthogonal reaction scheme between a tetrazine (Tz) and a trans-cyclooctene (TCO) was proposed as a means of linking ch735 to the cytotoxic maytansinoid DM1 that inhibits the assembly of microtubules (FIG. 13A). DM1 was chosen because it has been used successfully in other ADCs including T-DM1, an FDA-approved ADC for HER2-positive breast cancer (Lewis et al., “Targeting HER2-Positive Breast Cancer with Trastuzumab-DM1, an Antibody-Cytotoxic Drug Conjugate,” Cancer Res. 68:9280-90 (2008), which is hereby incorporated by reference in its entirety). Here, a trans-cyclooctene (TCO)-maleimide-DM1 non-cleavable drug linker was chemically synthesized (FIGS. 14A-14C). Following synthesis, TCO-maleimide-DM1 was conjugated to Tz-modified ch735, forming a 1,4-dihydropyridazine (Py) linkage between the two and typically resulting in drug-to-antibody ratios (DARs) of ˜2-3 (Table 2). To evaluate in vitro cytotoxicity, SH-SY5Y cells were treated with ch735-Py-DM1 and then examined cell viability. The ch735-Py-DM1 conjugate, but not the isotype-Py-DM1 control, showed polySia-specific cell killing of SH-SY5Y cells, and neither showed any cytotoxicity towards MCF7 cells (FIG. 13B). For comparison, a conjugate between Tz-modified trastuzumab and DM1 (T-Py-DM1) was similarly prepared, and it was found that it killed HER2-positive SKOV3 cells to an extent that was similar to ch735-Py-DM1 against SH-SY5Y cells (FIG. 13C). Importantly, the comparable target-specific potency that was measured for ch735-Py-DM1 relative to T-Py-DM1 (IC₅₀ values of 17 and 23 nM, respectively, Table 2) reveals the therapeutic potential of this glycan-directed ADC against polySia-positive cancers including neuroblastoma, small cell and non-small cell lung carcinomas, multiple myeloma, and Wilms' tumor.

TABLE 2
Summary of relevant antibody-drug conjugates

| Target | Antibody | Drug | DAR | EC₅₀ [nM] |
|---|---|---|---|---|
| polySia | ch735 | DM1 | 2-3 | 17* |
| HER2 | Trastuzumab | DM1 | 2-3 | 23* |
| HER2 | Trastuzumab | DM1 | 3-4 | 0.1-40^{2,3} |
| NCAM | Lorvotuzumab | DM1 | 3.5 | 0.2-5⁴ |
| NCAM | Promiximab | DUBA | 2 | 0.07-0.29⁵ |
| NCAM | Promiximab | MMAE | 3 | 0.3-20⁶ |
| NCAM | m906 | PBD | 4 | 0.00005-0.0017⁷ |
| sTn | various | MMAE | 3-5 | 0.5-11⁸ |
| sTn | 2G12-2B2 L0H3 | MMAE | 3-5 | 5-50⁹ |
| Tn | Chi-Tn | MMAF | 5 | ND¹⁰ |
| T | JAA-F11 | DM1 | 2-3.5 | 0.067-20¹¹ |
| Lewis Y | BR96 | DOX | 4 | 100-7000¹²⁻¹⁴ |

*this study; ND = not determined;
²Phillips et al., “Targeting HER2-Positive Breast Cancer with Trastuzumab-DM1, an Antibody-Cytotoxic Drug Conjugate,” Cancer Res. 68: 9280-90 (2008), which is hereby incorporated by reference in its entirety;
³Li et al., “A Biparatopic HER2-Targeting Antibody-Drug Conjugate Induces Tumor Regression in Primary Models Refractory to or Ineligible for HER2-Targeted Therapy,” Cancer Cell 29: 117-29 (2016), which is hereby incorporated by reference in its entirety;
⁴Whiteman et al., “Lorvotuzumab Mertansine, a CD56-Targeting Antibody-Drug Conjugate with Potent Antitumor Activity Against Small Cell Lung Cancer in Human Xenograft Models,” MAbs 6: 556-66 (2014), which is hereby incorporated by reference in its entirety;
⁵Yu et al., “Promiximab-Duocarmycin, a new CD56 Antibody-Drug Conjugates, is Highly Efficacious in Small Cell Lung Cancer Xenograft Models,” Oncotarget. 9: 5197-207 (2018), which is hereby incorporated by reference in its entirety;
⁶Yu et al., “Preparation and Anti-Cancer Evaluation of Promiximab-MMAE, an Anti-CD56 Antibody Drug Conjugate, in Small Cell Lung Cancer Cell Line Xenograft Models,” J. Drug Target 1-8 (2018), which is hereby incorporated by reference in its entirety;
⁷Feng et al., “Differential Killing of CD56-Expressing Cells by Drug-Conjugated Human Antibodies Targeting Membrane-Distal and Membrane-Proximal Non-Overlapping Epitopes,” MAbs 8: 799-810 (2016), which is hereby incorporated by reference in its entirety;
⁸Prendergast et al., “Novel Anti-Sialyl-Tn Monoclonal Antibodies and Antibody-Drug Conjugates Demonstrate Tumor Specificity and Anti-Tumor Activity,” MAbs 9: 615-27 (2017), which is hereby incorporated by reference in its entirety;
⁹Eavarone et al., “Humanized Anti-Sialyl-Tn Antibodies for the Treatment of Ovarian Carcinoma,” PLoS ONE 13: e0201314 (2018), which is hereby incorporated by reference in its entirety;
¹⁰Sedlik et al., “Effective Antitumor Therapy Based on a Novel Antibody-Drug Conjugate Targeting the Tn Carbohydrate Antigen,” Oncoimmunology 5: e1171434 (2016), which is hereby incorporated by reference in its entirety;
¹¹Tati et al., “Humanization of JAA-F11, a Highly Specific Anti-Thomsen-Friedenreich Pancarcinoma Antibody and in vitro Efficacy Analysis,” Neoplasia 19: 716-33 (2017), which is hereby incorporated by reference in its entirety;
¹²Sjogren et al., “Antitumor Activity of Carcinoma-Reactive BR96-Doxorubicin Conjugate Against Human Carcinomas in Athymic Mice and Rats and Syngeneic Rat Carcinomas in Immunocompetent Rats,” Cancer Res. 57: 4530-6 (1997), which is hereby incorporated by reference in its entirety;
¹³Trail et al., “Cure of Xenografted Human Carcinomas by BR96-Doxorubicin Immunoconjugates,” Science 261: 212-5 (1993), which is hereby incorporated by reference in its entirety;
¹⁴Wahlet al., “Selective Tumor Sensitization to Taxanes with the mAb-Drug Conjugate cBR96-Doxorubicin,” Int. J. Cancer 93: 590-600 (2001), which is hereby incorporated by reference in its entirety.

### Example 5—Materials and Methods

Construction of chimeric human mAb ch735.

The DNA sequences for the V_{H} and V_{L} domains of mAb mo735 (Nagae et al., “Crystal Structure of Anti-Polysialic Acid Antibody Single Chain Fv Fragment Complexed with Octasialic Acid: Insight into the Binding Preference for Polysialic Acid,” J. Biol. Chem. 288:33784-96 (2013), which is hereby incorporated by reference in its entirety) were obtained from the GenBank™/EBI Data Bank (accession number AB821355) and ordered from GeneArt Gene Synthesis (Thermo Fisher Scientific). The variable regions of mAb 735 were then swapped with the existing variable regions in pVITRO1-Trastuzumab-IgG1/k (Addgene plasmid #61883) as previously described to generate the vector pVITRO-735-IgG1/k (Dodev et al., “A Tool Kit for Rapid Cloning and Expression of Recombinant Antibodies,” Sci. Rep. 4:5885 (2014), which is hereby incorporated by reference in its entirety). Briefly, polymerase incomplete primer extension (PIPE) PCR was performed using sets of primers (Table 3) to generate four linear fragments of the construct with 5′ PIPE overhangs. All cloned plasmids were confirmed by DNA sequencing.

TABLE 3
PIPE cloning primers used to generate mAb ch735

| | | SEQ |
|---|---|---|
| Primer | Sequence (5′-3′) | ID NO: |
| Linear_Kfwd | cgtacggtggcggcgccatc | 7 |
| | tgtcttcatcttcccgccat | |
| Linear_Hrev | ggagtgcgcgcctgtggcgg | 8 |
| | ccgccaccaagaagaggatc | |
| Linear_Hfwd | Gctagcacacagagcccatc | 9 |
| | cgtcttccccttgacccgct | |
| Linear_Krev | accgcggctagctggaaccc | 10 |
| | agagcagcagaaacccaatg | |
| 735_Kfwd | gggttccagctagccgcggt | 11 |
| | gatgtagtcatgacgcagac | |
| 735_Krev | gggactcgcctggaaatcaaa | 12 |
| | cgtacggtggcggcgccatc | |
| 735_Hfwd | ccgccacaggcgcgcactcc | 13 |
| | cagattcagctgcagcaatc | |
| 735_Hrev | ccagcgtaaccgtgtcatcc | 14 |
| | gctagcaccaagggcccatc | |

Cell Culture and Reagents.

Production of recombinant mAbs was performed using FreeStyle™ 293-F cells (ThermoFisher Scientific). FreeStyle™293-F cells were maintained in FreeStyle 293 expression medium (ThermoFisher Scientific). Cancer cell lines SH-SY5Y, H82, H69, K562, MCF7, and SKOV3 were obtained from American Type Culture Collection (ATCC) while cell lines SW2 and A549 were kindly provided by Dr. Karen Colley (University of Illinois at Chicago). SH-SY5Y cells were maintained in high glucose DMEM/F12 medium supplemented with 10% Hyclone FetalClone I serum (VWR), 1% MEM non-essential amino acids solution (ThermoFisher Scientific), penicillin (100 U/mL) and streptomycin (100 μg/mL) (ThermoFisher Scientific). H82, H69, K562, and A549 cells were maintained in RPMI 1640 with L-glutamine (ThermoFisher Scientific) supplemented with 10% Hyclone FetalClone I serum, penicillin (100 U/mL) and streptomycin (100 μg/mL). MCF7 cells were maintained in high glucose DMEM supplemented with 10% Hyclone FetalClone I serum, insulin (10 μg/mL, Sigma), penicillin (100 U/mL) and streptomycin (100 μg/mL). SKOV3 and SW2 cells were maintained in high glucose DMEM supplemented with 10% Hyclone FetalClone I serum, penicillin (100 U/mL) and streptomycin (100 μg/mL). All cell lines were maintained at low passage numbers and routinely checked for mycoplasma by PCR as previously described (Young et al., “Detection of Mycoplasma in Cell Cultures,” Nat. Protoc. 5:929-34 (2010), which is hereby incorporated by reference in its entirety).

Expression and Purification of Ch735 and Trastuzumab.

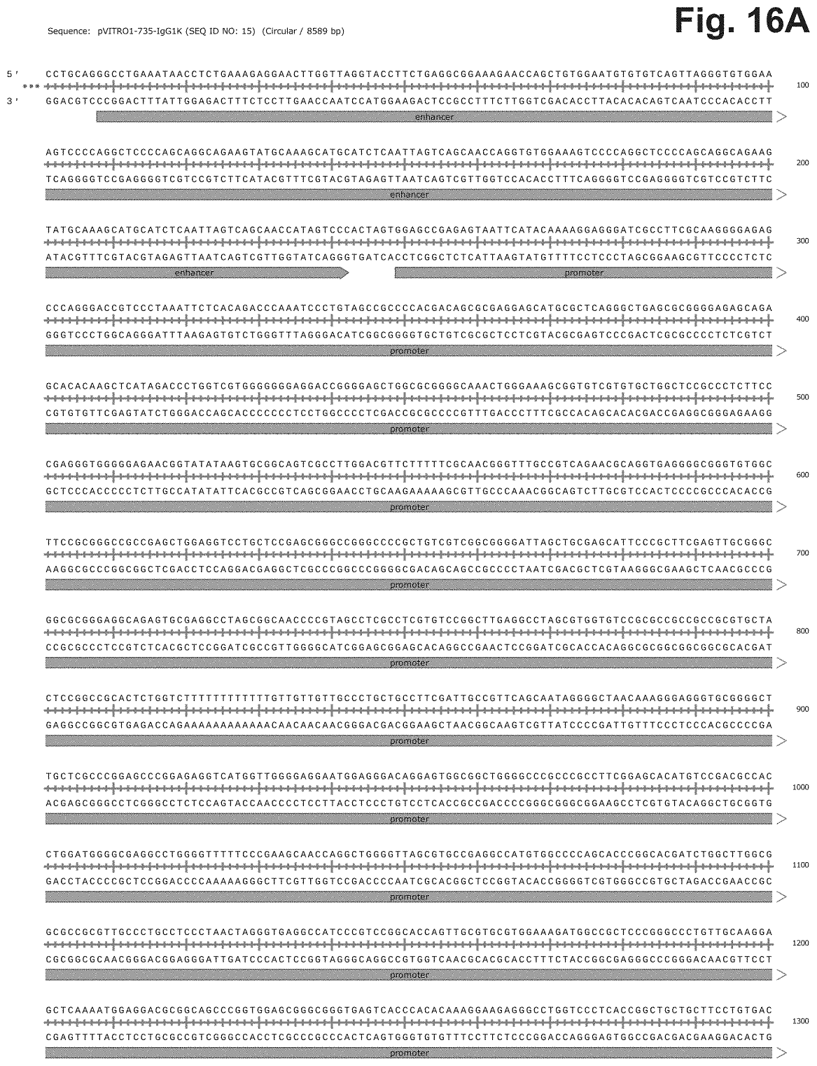

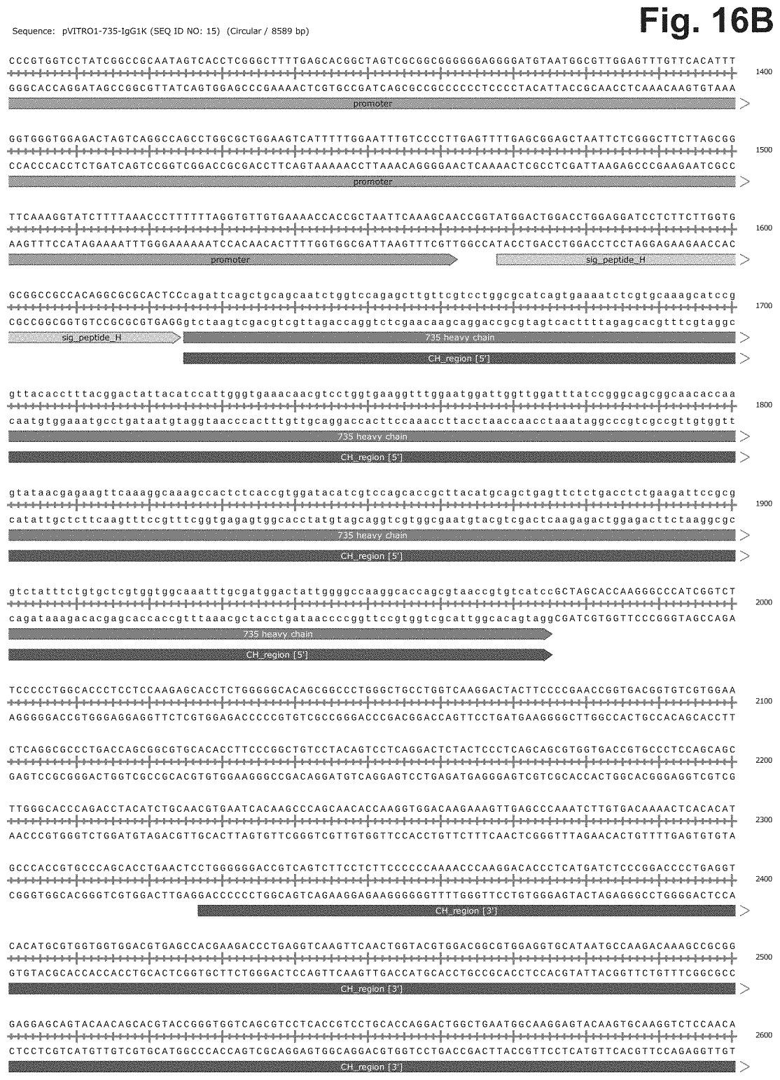

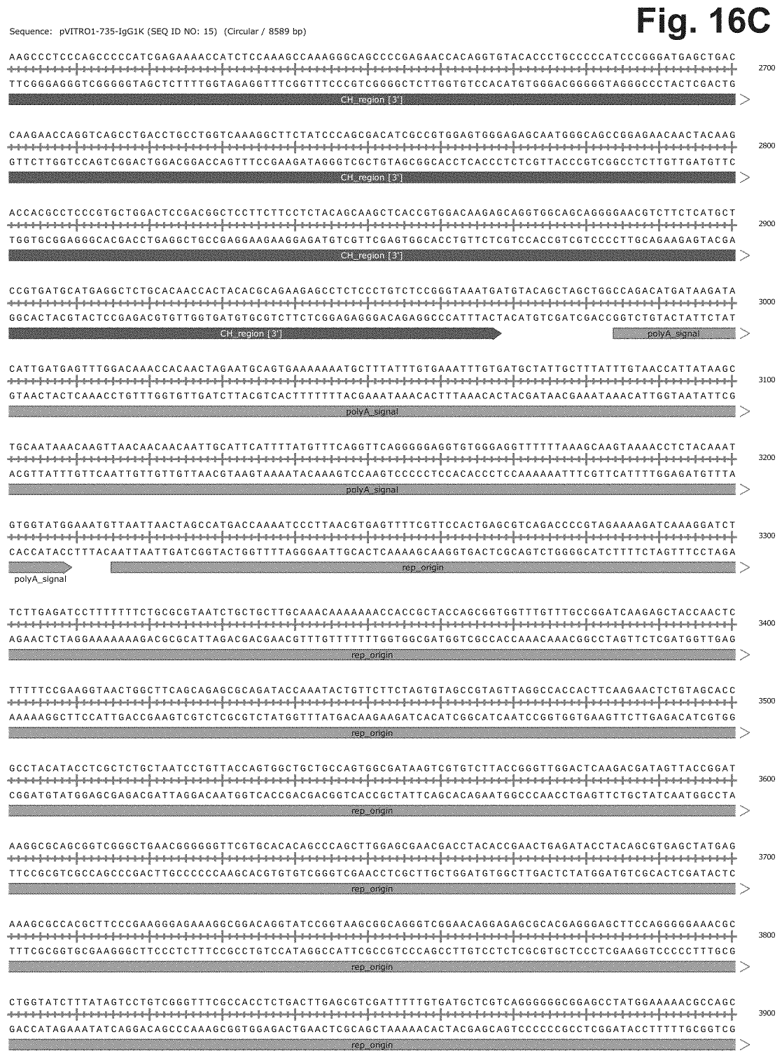

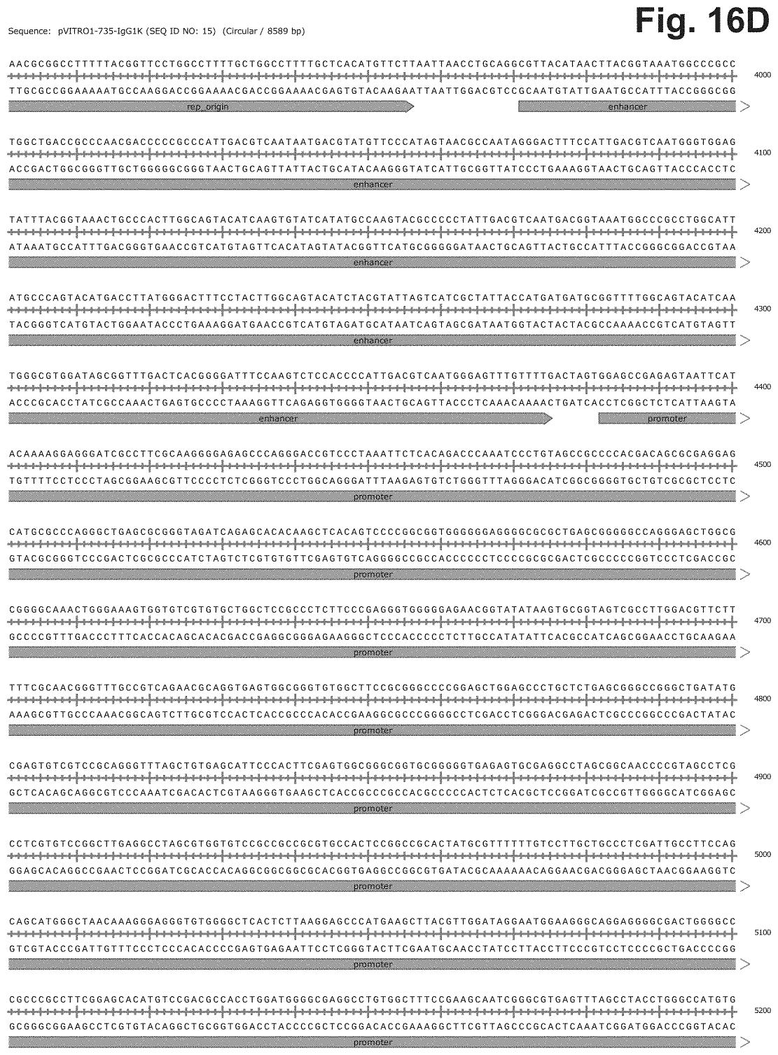

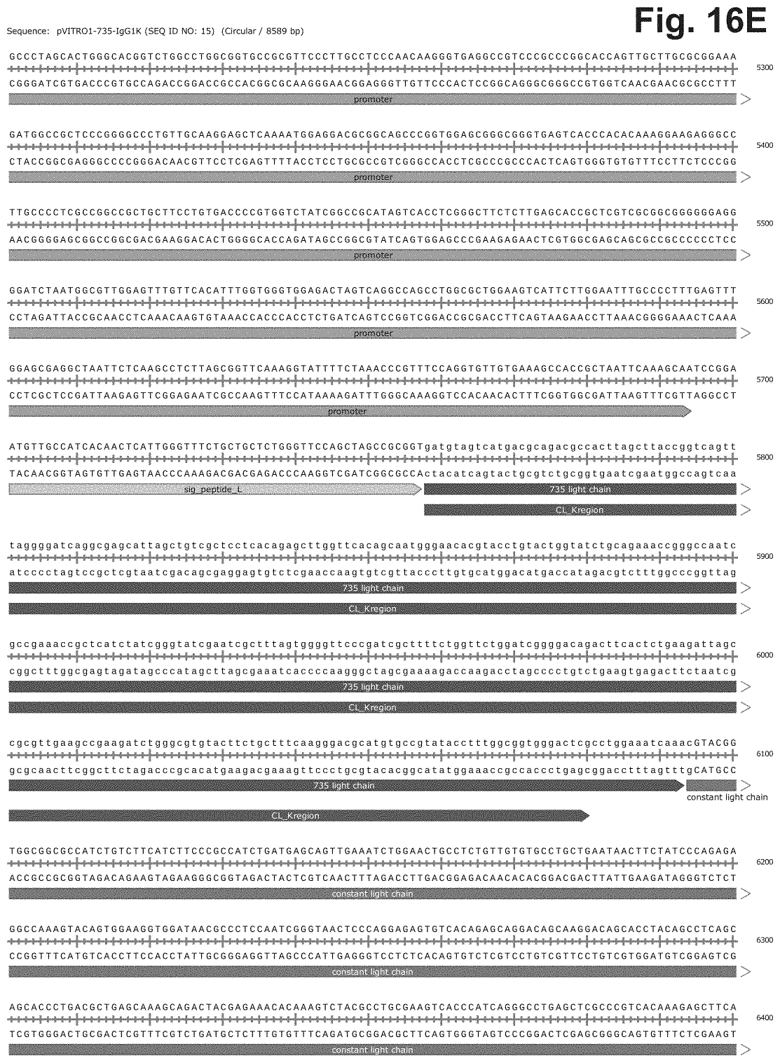

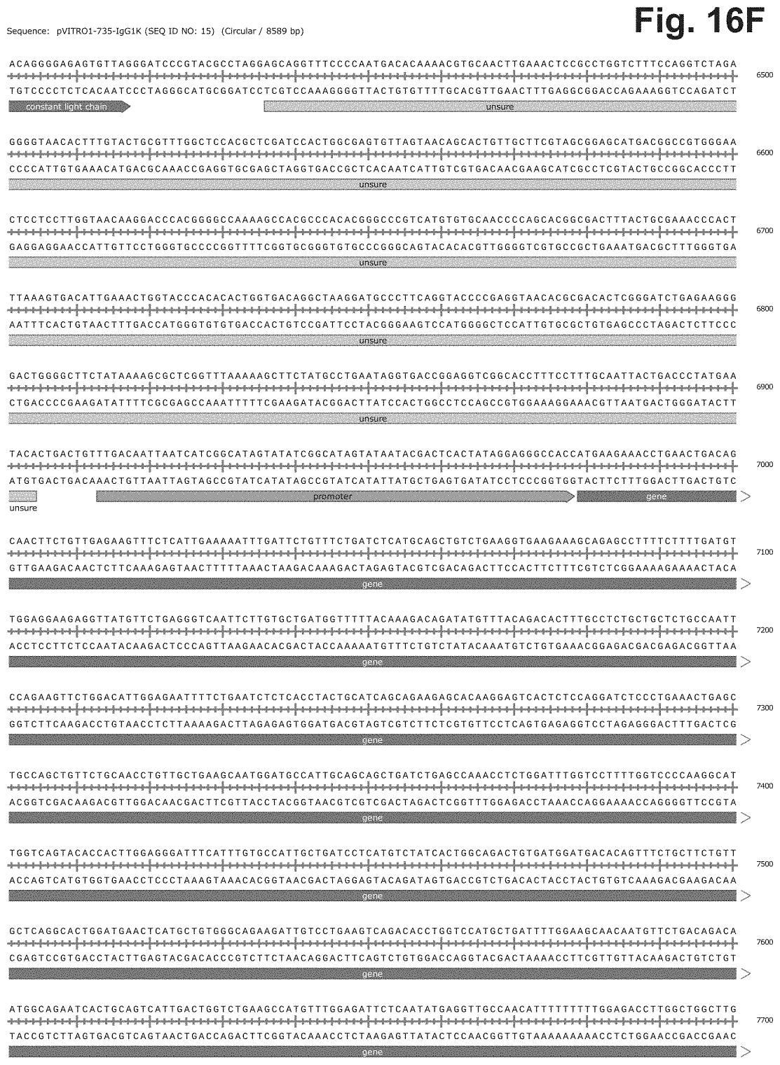

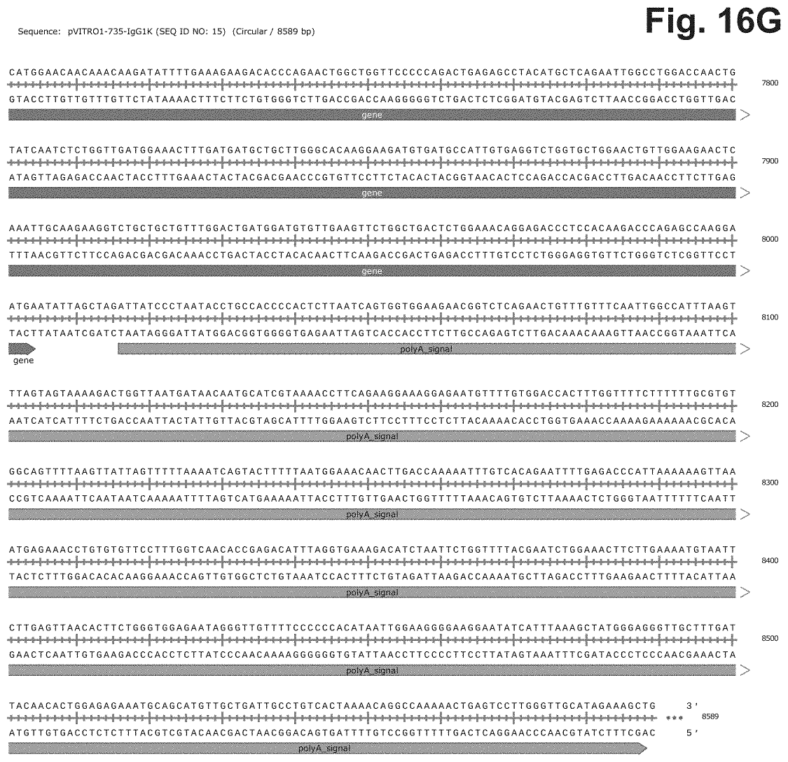

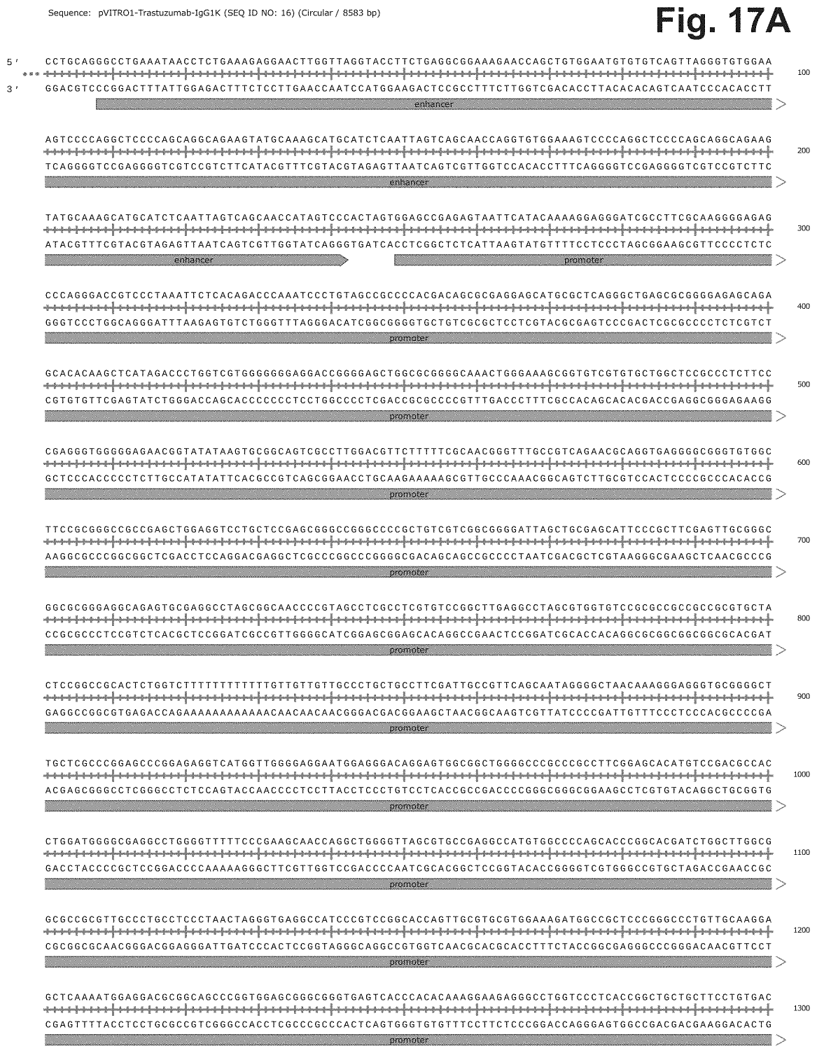

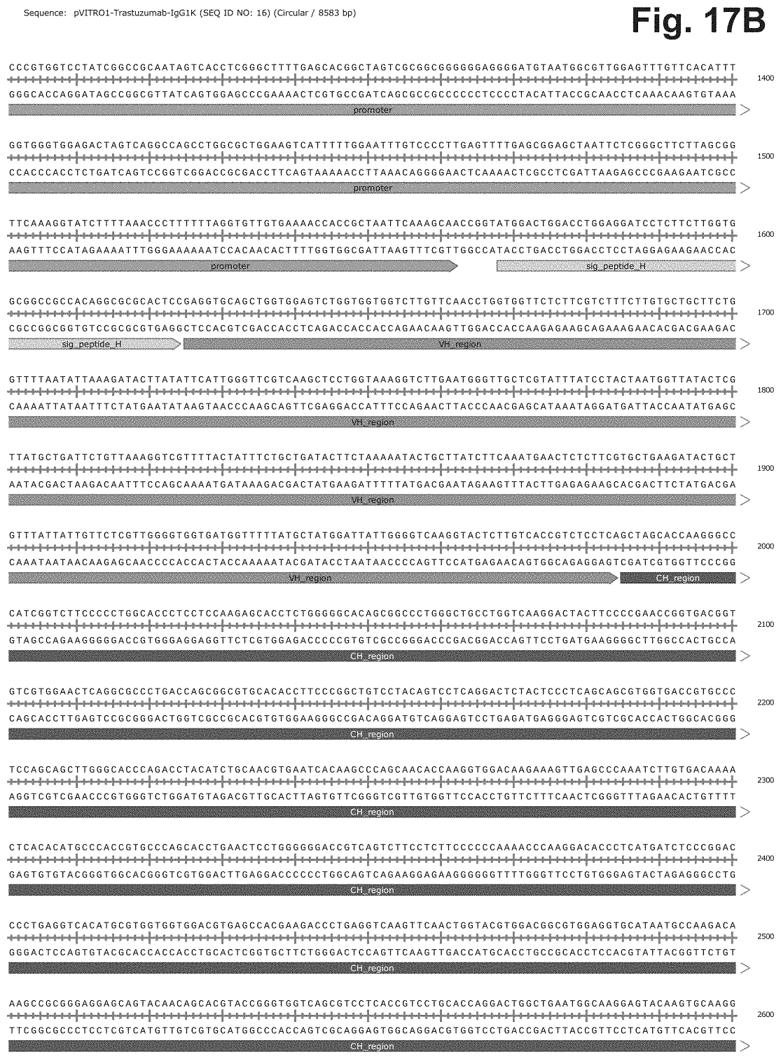

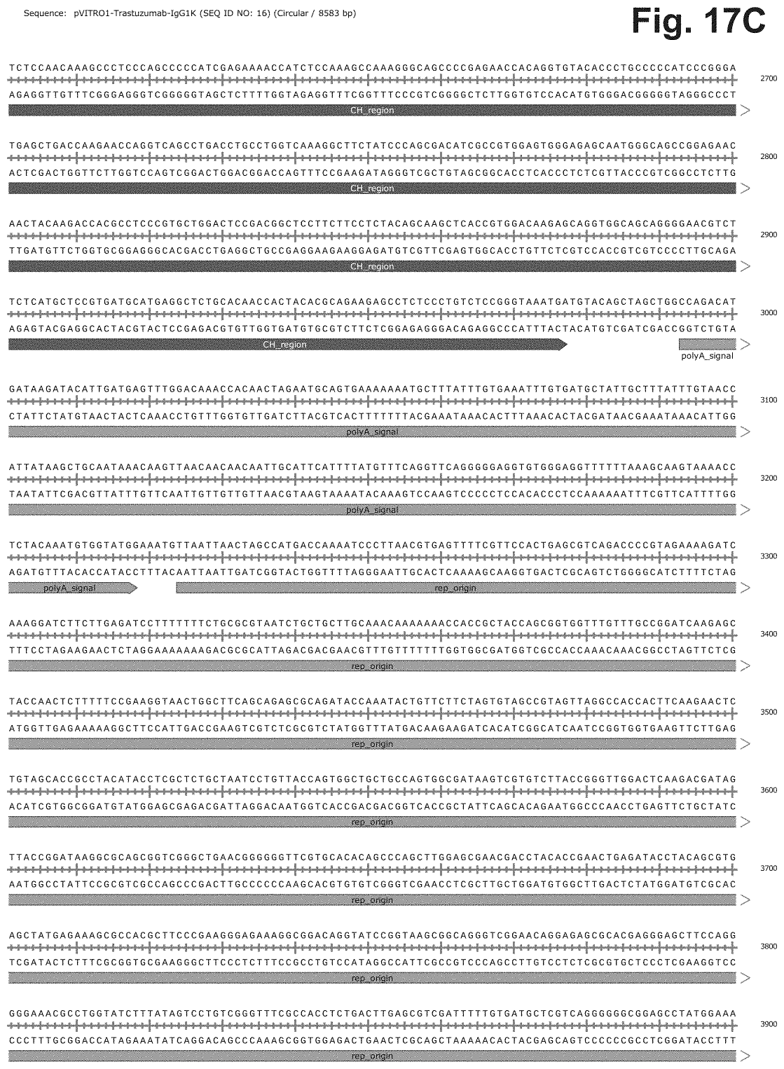

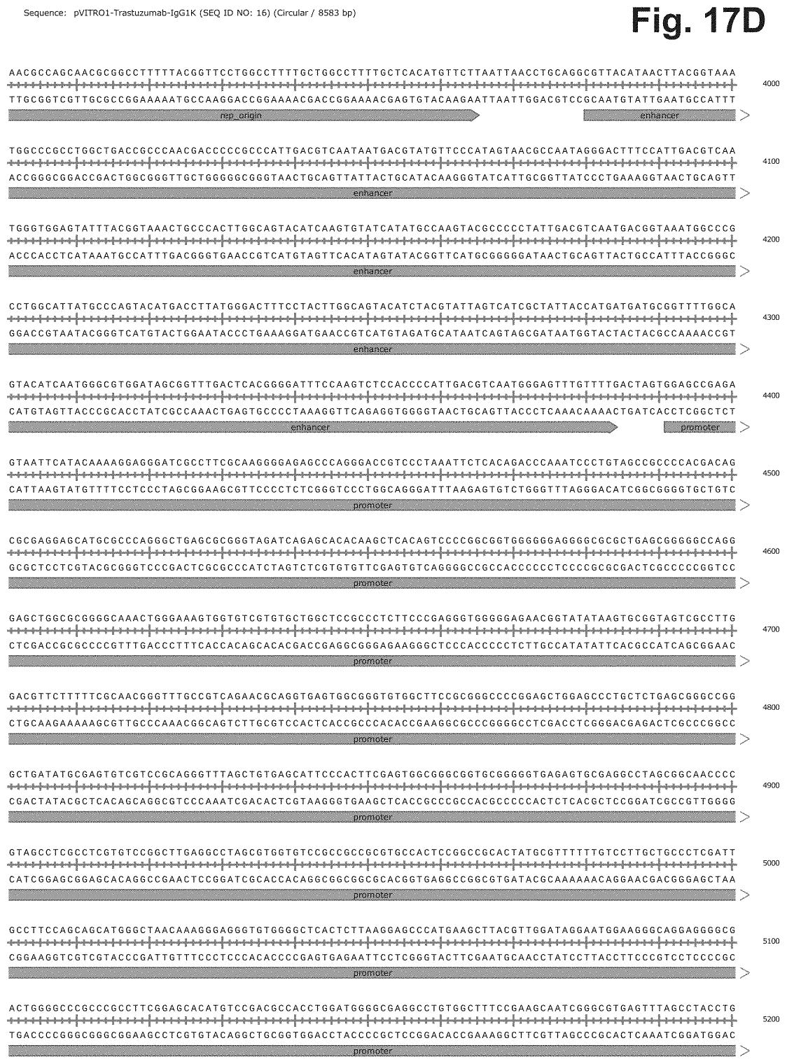

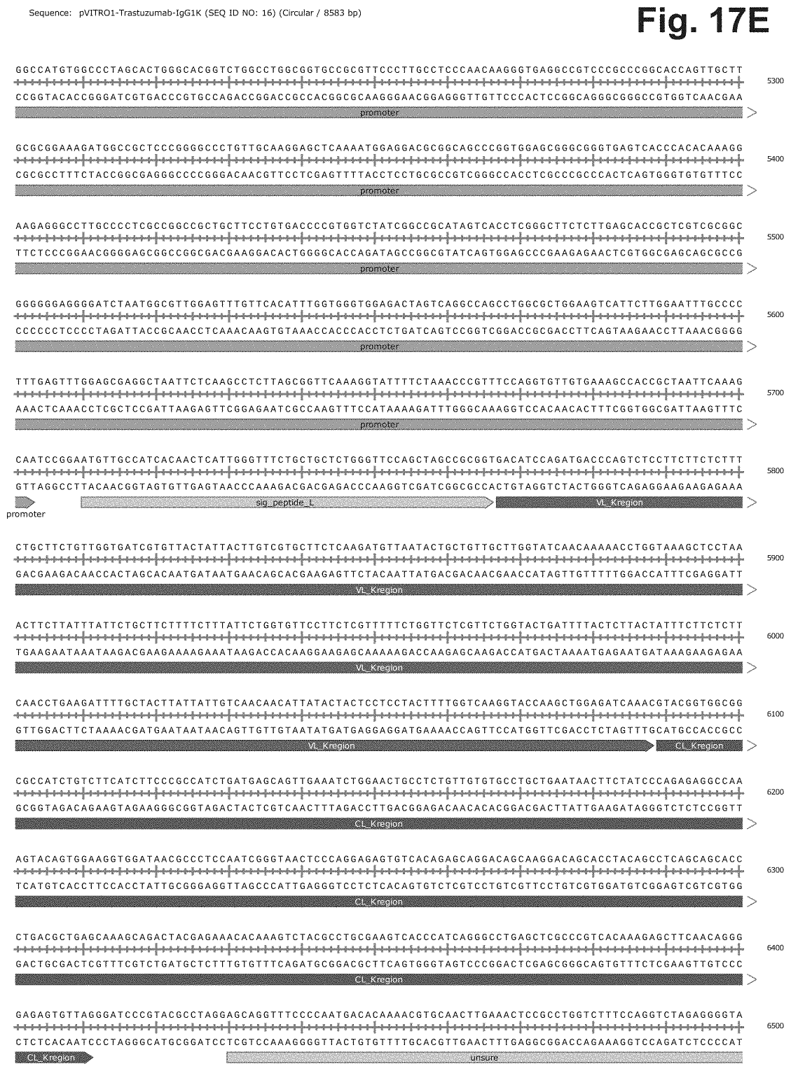

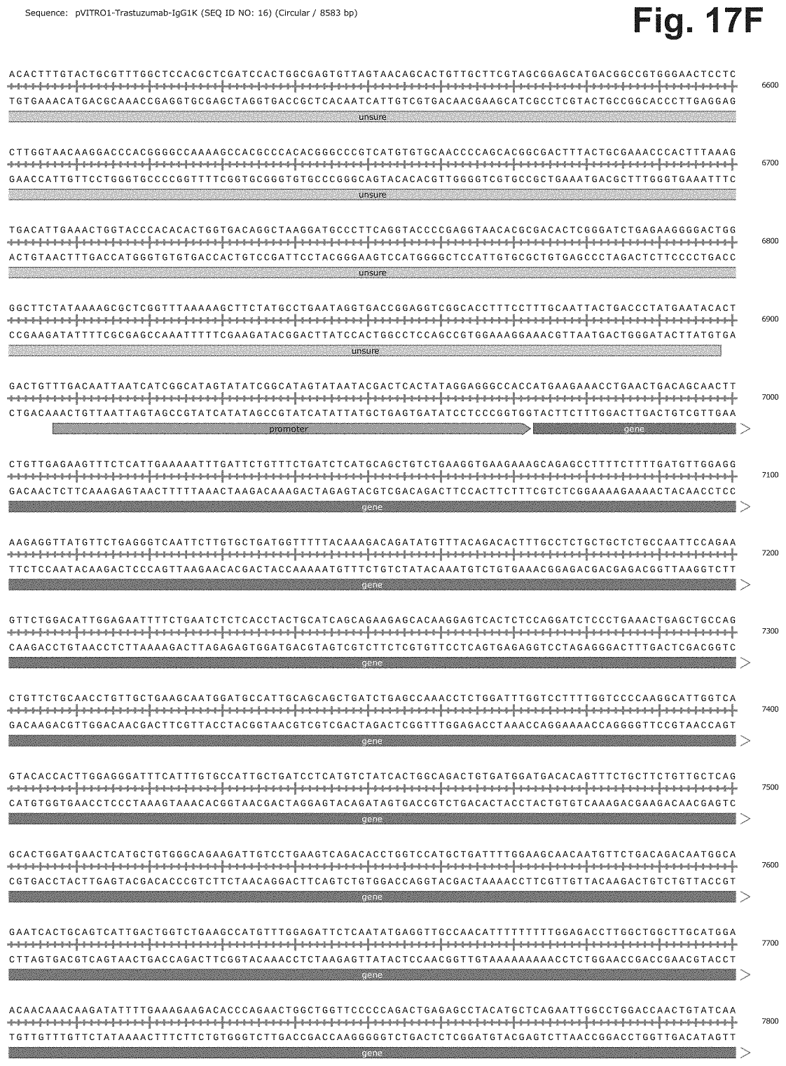

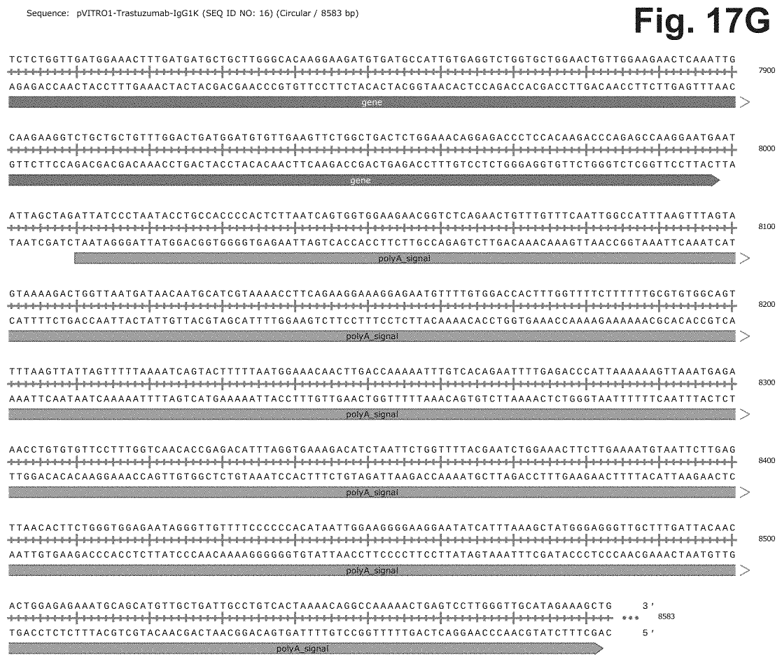

293-F cells cultured in FreeStyle™ 293 Expression Medium (ThermoFisher Scientific) were transfected with pVITRO-735-IgG1/k (SEQ ID NO: 15; FIGS. 16A-16G), or pVITRO1-Trastuzumab-IgG1/k (SEQ ID NO: 16; FIGS. 17A-17G), using FreeStyle™MAX transfection reagent (ThermoFisher Scientific) according to the manufacturer's instructions and selected under hygromycin B as previously described (Dodev et al., “A Tool Kit for Rapid Cloning and Expression of Recombinant Antibodies,” Sci. Rep. 4:5885 (2014), which is hereby incorporated by reference in its entirety). Purified plasmid DNA was precipitated by mixing 1/10 the volume of 3 M sodium acetate pH 5.2 and 2-3 volumes of 100% ethanol and freezing at −80° C. for 2 h. The DNA was collected by centrifugation at 13,000×g at 4° C. for 30 min and resuspended in 100 μL of sterile tissue culture grade water (Thermo Fisher). After selection, cultures were expanded to 1 L culture volume and maintained with 50% hygromycin B (25 μg/mL). Supernatants were harvested every 48 h, centrifuged at 1000×g for 15 min, passed over 0.2 μm filters (VWR) and stored at 4° C. until use.

Protein A/G agarose (Thermo Fisher) was used to purify antibodies from the supernatant according to the manufacturer's recommendations. The agarose equilibrated with 10 mL phosphate-buffered saline (PBS) in a polypropylene gravity column. The supernatant was then allowed to completely pass through the column. The column was then washed with PBS until there was no signal in the flow through at an absorbance of 280 nm (Abs₂₈₀). Antibodies were eluted from the column with 0.1 M glycine-HCl (pH 2.0) in 1-mL fractions and neutralized with 100 μL 1 M Tris (pH 8.0). Antibody purity was evaluated by SDS-polyacrylamide gel electrophoresis (SDS-PAGE) under reducing and non-reducing conditions and visualized by staining with Coomassie Blue G-250. Protein A/G-purified antibodies were analyzed by size exclusion chromatography (SEC) on a 4.6 mm ID×30 cm TSKgel SuperSW3000 SEC column with 4-μm particles. Pure antibodies were eluted from the column at 8.66 min in 0.1 M phosphate buffer containing 0.1 M Na₂SO₄, pH 6.7.

ELISA.

Costar 96-well ELISA plates (Corning) were coated overnight at 4° C. with 50 μl of 1 μg/mL chicken brain NCAM (Millipore) or endoN-treated NCAM in PBS. Chicken NCAM (Millipore, AG265) was digested with 1.5 μg of endoN per 50 μg of NCAM overnight at 37° C. After blocking with 5% (w/v) milk in PBS for 1-3 h at room temperature or overnight at 4° C., ELISA plates were washed three times with wash buffer (PBST with 0.3% BSA) and incubated with serially diluted purified ch735, mo735 (Absolute Antibody), or ab5032 (Millipore) for 1 h at room temperature. Antibody samples were quantified with a Nanodrop. After washing three times with wash buffer, 100 μl of 1:5,000-diluted rabbit anti-human IgG (Fc) antibody-HRP conjugate (Thermo Fisher) or goat anti-rabbit IgG-HRP (Abcam) in wash buffer was added to each well for 1 h. Plates were washed and developed using standard protocols.

Specificity Profiling Using Glycan and Glycoprotein Arrays.

Specificity of mo735 and ch735 was determined using printed glycan arrays 5.3 and 5.4 at the CFG. Both antibodies were analyzed at 10 μg/mL with 5 μg/mL of anti-mouse or anti-human Alexa-Fluor 647 (AF647)-conjugated secondary, respectively. Specificity of mo735 (1 μg/mL) and ch735 (10 μg/mL) was also assessed on a custom glycoprotein array that contained ˜40-50 glycoproteins including chicken brain NCAM and endoN-treated chicken brain NCAM.

SPR.

Equilibrium binding-affinity measurements were made by SPR analysis on a Biacore 3000 system. Antibodies mo735 and ch735 were bound to the surface of a Protein A sensor chip with a target level of 1700 response units (RUs). Serial dilutions of the antigen, chicken NCAM, prepared in 10 mM HEPES, pH 7.4, 150 mM NaCl, 3 mM EDTA, 0.005% polysorbate-20 (HBS-EP buffer, GE Healthcare) at concentrations ranging from 0.25 to 250 nM were injected over the chip using the same buffer at a flow rate of 20 μl/min (10 min injection time, 2 min stabilization time, 20 min dissociation). The surface of the chip was regenerated between the injections of each serial dilution with 10 mM glycine, pH 1.5 (30 sec injection time, 3 min stabilization time). Kinetic parameters were determined by fitting the maximum response values for each concentration using the Hill slope non-linear regression analysis in Prism software.

CRISPR/Cas9 Genome Editing.

CRISPR guide RNAs targeting ST8SiaII (crRNA1: ATGCAGTGCGCACGTTGACG; SEQ ID NO: 5) and ST8Sia4 (crRNA1: ACCCGATGAGTTGCGTCTCC; SEQ ID NO: 6) were purchased from Genscript in the pLentiCRISPR v2 vector. Knockout cell lines were generated in SW2 cells using protocols described by Zhang and colleagues (Shalem et al., “Genome-Scale CRISPR-Cas9 Knockout Screening in Human Cells,” Science 343:84-7 (2014), which is hereby incorporated by reference in its entirety). Briefly, after lentiviral transduction, cells were maintained under selection with 1 μg/mL puromycin for at least 14 days prior to analysis by flow cytometry.

Flow Cytometric Analysis.

Cancer cells were trypsinized and collected with media, followed by three washes in PBS. To remove polySia, cells were treated with endoN at 3 μg/mL in PBS for 1 h at room temperature. The cells are resuspended in 4% paraformaldehyde (PFA), fixed at room temperature with constant agitation for 10 min, and then washed two times with PBS, and two times with 0.5% BSA in PBS. The cells were collected and resuspended to 1 million cells/100 μL and pipetted into a round bottom 96-well plate. The cells were pelleted in the 96-well plate and resuspended in 0.5% BSA in PBS containing mo735 or ch735 (5 μg/mL) and incubated for 30 min at room temperature with constant agitation. Cells were washed three times with 0.5% BSA in PBS and resuspended in anti-mouse IgG-Alexa-Fluor 488 (AF488) secondary or anti-human IgG-AF488 secondary (ThermoFisher Scientific) at a 1:200 dilution for 30 min at room temperature in the dark with constant agitation. Cells were washed three times, resuspended in 500 μL of 0.5% BSA in PBS, and analyzed on a BD FACSCalibur flow cytometer using Cell Quest Pro software (BD Biosciences).

Confocal Microscopy.

Adherent cells were plated at 20,000 cells/cm² on poly-L-lysine coated 35-mm glass bottom dishes and adhered overnight. To remove polySia, cells were then treated with endoN at 3 μg/mL in cell culture media overnight. Suspension cells were collected on the day of the experiment and labeled in suspension with the same protocol as the adherent cells. Cells were fixed with 4% paraformaldehyde and subsequently blocked with 5% normal goat serum PBS (NPBS) for 1 h at room temperature. Antibodies mo735 and ch735 were diluted to 5 μg/mL in 5% NPBS and incubated overnight at 4° C. Anti-mouse and anti-human AF488-conjugated secondary antibodies A32723 and A11013 (ThermoFisher Scientific) were diluted 1:200 in NPBS and incubated for 2 h at room temperature. Wheat germ agglutinin-AF647 (WGA-647) was diluted to 1 μg/mL in NPBS and incubated for 10 min at room temperature. Hoescht dye was used at 1 μg/mL in PBS for 5 min at room temperature. Samples were imaged on a Zeiss LSM inverted 880 confocal microscope using a 40× water immersion objective.

IHC.

The avidin-biotin complex (ABC) immunoperoxidase method was performed essentially as previously described (Zhang et al., “Selection of Tumor Antigens as Targets for Immune Attack Using Immunohistochemistry: I. Focus on Gangliosides,” Int. J. Cancer 73:42-9 (1997), which is hereby incorporated by reference in its entirety). Briefly, the sections were quenched with 0.1% H₂O₂ in PBS for 15 min, blocked with avidin and biotin reagents (Vector, Burlingame, Calif.) for 10 min each, incubated in 10% serum from which the second antibody was raised and incubated with mAb 735 at 1 μg/ml for 1 h. This concentration was selected based on strong reactivity against known positive target cells and little or no background against stroma. The sections were subsequently incubated with biotinylated secondary antibodies for 30 min, and then incubated in ABC reagent per manufacturer's protocols (ABC Kit, Vector Laboratories, PK-6102) for 30 min. Reactions were developed with liquid DAB+Substrate Chromogen System (Dako, cat # K3468) for 3 min at room temperature. Slides were then counterstained with Mayer's Hematoxylin (Dako Cyomation, cat # S3309) for 1 min at room temperature. The immunoreactivities were graded based on the percentage of positive cells and staining intensity above that seen on the negative control. Known positive and negative control slides were used in each experiment.

Internalization Assays.

To calculate percent internalization, pre-chilled cells were incubated with 50 nM ch735 on ice for 1 h and then washed to remove unbound antibodies. For each time point, one aliquot of cells remained on ice and one was incubated at 37° C. for 15, 30, or 60 min. Cells were fixed in 2% paraformaldehyde for 20 min and then stained with AF488-labeled antibody against human IgG and analyzed by flow cytometry and FlowJo software. Receptor-antibody complex internalization was calculated using the geometric mean as percent fluorescent intensity loss at 37° C. relative to that on ice. For each sample, the geometric mean fluorescence intensity (MFI) of 10,000 cells was measured in triplicate.

Colocalization Microscopy.

SH—SY5Y or SW2 cells were plated at 20,000 cells/cm² on poly-L-lysine coated 35 mm glass bottom dishes and adhered overnight. To remove polySia, cells were treated as described above. To measure receptor-antibody internalization, cells were incubated with 150 nM AF488 labeled ch735, human IgG isotype control (ThermoFisher Scientific), mo735, or mouse IgG isotype control (anti-MBP mAb, NEB) and 100 nM AF647 transferrin or AF647 anti-LAMP-3 antibody (Santa Cruz) for 1 h. Cells were washed and then fixed as described above. To examine lysosomal trafficking, cells were incubated with 150 nM of ch735 or isotype at 37° C. for 2 h, washed, fixed, and then permeabilized using 0.1% Triton X-100 NPBS. Cells were stained with AF488-labeled antibody against human IgG to visualize antigen-antibody complex (ThermoFisher Scientific) and mouse anti-human LAMP-1 clone D2D11 (Cell Signaling) followed by AF647-labeled anti-rabbit IgG to visualize the lysosomes (ThermoFisher Scientific). Hoescht dye was used at 1 μg/mL in PBS for 5 min at room temperature. Samples were imaged on a Zeiss LSM inverted 880 confocal microscope using a 40× water immersion objective. For colocalization analysis, a 5-μM line was drawn across the apparent vesicles. The fluorescence intensity of the plot profile was analyzed using FIJI software. Fluorescence intensity was normalized to the maximum value for each channel.

Drug linker synthesis.

To synthesize the drug linker, 650 μg of DM1 was incubated at 1.1 mM with 3 molar equivalents of maleimide-PEG3-TCO (Click Chemistry Tools) in 3:1 DMSO:PBS overnight at room temperature. The reaction mixture was purified on a C18 analytical RP-HPLC column on a gradient of 5-95% acetonitrile in water over 30 min. The product was dried, resuspended in DMSO, and quantified via Abs₂₅₂ measurements (ext. coeff_{252 nm}=26,790 M cm⁻¹). Product mass was verified via LCMS (expected mass=1,260.56).

ADC conjugation. Purified ch735, purified trastuzumab, or human IgG1 isotype control (ThermoFisher Scientific) was reacted with 10 molar equivalents of methyltetrazine-PEG4-NHS ester overnight at 37° C. Excess reagent was removed by centrifugation dialysis. The Tz-conjugated antibody was then incubated with 3 molar equivalents of TCO-maleimide-DM1 drug linker for 5 h at 37° C. Excess reagent was removed by centrifugation dialysis. Average DAR was determined using absorbance spectroscopy to calculate the concentrations of antibody and drug (Chen Y., “Drug-to-Antibody Ratio (DAR) by UV/Vis Spectroscopy,” Methods Mol. Biol. 1045:267-73 (2013), which is hereby incorporated by reference in its entirety). The following previously established extinction coefficients were used for each component: ε280 DM1=5,700 M⁻¹ cm⁻¹, c252 DM1=26,790 M⁻¹ cm⁻¹ and ε280 Antibody=218,134 M⁻¹ cm⁻¹, c252 Antibody=76,565 M⁻¹ cm⁻¹ (Kim et al., “Statistical Modeling of the Drug Load Distribution on Trastuzumab Emtansine (Kadcyla), a Lysine-Linked Antibody Drug Conjugate,” Bioconjug. Chem. 25:1223-32 (2014), which is hereby incorporated by reference in its entirety).

Cell Viability Assay.

SKOV3, MCF7, and SH-SY5Y cells were plated at 5,000, 2,500, and 2,500 cells/well, respectively, and allowed to rest for 24 h. Five-fold serial dilutions of the antibodies were added starting at 150 nM and incubated for 72-144 h. The viability assays were then developed using Alamar blue according to manufacturer's protocol (Bio-Rad). Percent viability is calculated by first subtracting the value of media alone from all samples. Subsequently, the resulting values are divided by the values measured from an un-treated control representing maximum viability.

Western Blot Analysis.

For SDS-PAGE analysis, samples were prepared under reducing (with 5% β-mercaptoethanol) and non-reducing conditions with 4× Laemmli sample buffer (BioRad). In both cases, samples were heated at 100° C. for 10 min and then loaded on 4-20% tris-glycine gels (Bio-Rad). Following electrophoresis, resolved proteins were transferred to polyvinylidene fluoride (PVDF) membranes (Millipore).

Membranes were rinsed with PBS and then blocked with 5% milk (w/v) in PBS containing 0.05% Tween 20 (PBST) for 1 h. After three washes with PBST, membranes containing chimeric IgGs were probed with 1:5000-diluted rabbit anti-human Fc-horseradish peroxidase (HRP) conjugate (ThermoFisher Scientific) or anti-human kappa light chain-HRP conjugate (ThermoFisher Scientific). After washing three more times with PBST, membranes were incubated with Clarity ECL Western Blotting Substrate (Bio-Rad) and then visualized using a Bio-Rad Chemidoc XRS+.

Supplementary Internalization Assays.

For supplementary internalization assays, AF488-labeled isotype and mo735 antibodies were incubated with trypsinized SW2 cells at various concentrations for 2 h. Upon measurement in the flow cytometer, the extracellular fluorescence was quenched using trypan blue (final concentration 0.2%). For each sample, the mean fluorescence intensity (MFI) of 10,000 cells was measured in triplicate. Additionally, to assess antibody internalization, mo735 was labeled with pHrodo Green (ThermoFisher Scientific) and then incubated with SW2 cells for 40 min. After incubation, cells were washed and fixed with 4% PFA for 10 min at RT. Wheat germ agglutinin-AF647 (WGA-647) was diluted to 1 μg/mL in NPBS and incubated for 10 min at room temperature. Hoescht dye was diluted to 1 μg/mL in PBS and incubated for 5 min at room temperature. Samples were imaged on a Zeiss LSM inverted 880 confocal microscope using a 40× water immersion objective. Antibody internalization was also assessed according to the protocol described by Schmitz and coworkers (Diestel et al., “NCAM is Ubiquitylated, Endocytosed and Recycled in Neurons,” J. Cell. Sci. 120:4035-49 (2007), which is hereby incorporated by reference in its entirety), cells were incubated for 1 or 4 h at 37° C. with 150 nM mo735 in cell culture media. Cells were rinsed with 4° C. media, fixed for 10 min at 4° C. with 4% PFA in PBS, and incubated 30 min with AF488-conjugated anti-mouse antibody (1:200 in 5% NPBS) for the detection of cell surface-associated polySia. After washing, cells were incubated with unconjugated rabbit anti-mouse immunoglobulins (0.25 mg/mL in 5% NPBS) overnight at 4° C. to saturate all binding sites of the first antibody. Next, cells were post-fixed 5 min with 4% PFA at 4° C. and permeabilized with 0.5% Triton X-100 for 20 min. Internalized mo735 was visualized using AF647 anti-mouse antibodies (1:200). Hoescht dye was diluted to 1 μg/mL in PBS and incubated for 5 min at room temperature. Samples were imaged on a Zeiss LSM inverted 880 confocal microscope using a 40× water immersion objective.

Internalization of a polySia-Specific Single-Chain Fv (scFv735)

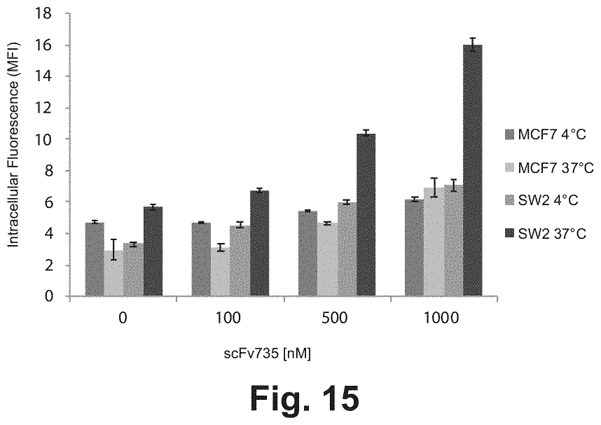

A polySia-specific single-chain Fc (scFV735) was constructed by genetically fusing together the DNA encoding the variable heavy (V_{H}) and variable light (V_{L}) genes derived from mo735 with a flexible GlySer linker. Alexa Fluor 488 labeled scFv735 was incubated with SW2 (polySia positive externally) and MCF7 (polySia negative externally) cells for 2 hours at different concentrations ranging from 0-1,000 nM at either 4° C. or 37° C. (internalization). After incubation, the cells were washed and extracellular fluorescence was quenched with 0.2% Trypan Blue. Intracellular Fluoresence (geometric mean fluorescence intensity, MFI) indicative of internalized scFv735 was measured by flow cytometery. Akin to the results with the mAb, scFv735 was observed to internalize in SW2 cells that express polySia on their surface but not MCF7 cells that do not display the polySia antigen on their surface (FIG. 15). Internalization was observed to increase with increasing concentration of scFv735 and was blocked when the temperature was reduced to 4° C., indicating an endocytic mechanism (FIG. 15).

The nucleic acid sequence of scFV735 (GenBank Accession No. AB821355.1; SEQ ID NO: 17) is as follows:

```
    1 GATGTAGTCA TGACGCAGAC GCCACTTAGC TTACCGGTCA GTTTAGGGGA TCAGGCGAGC
  61 ATTAGCTGTC GCTCCTCACA GAGCTTGGTT CACAGCAATG GGAACACGTA CCTGTACTGG
121 TATCTGCAGA AACCGGGCCA ATCGCCGAAA CCGCTCATCT ATCGGGTATC GAATCGCTTT
181 AGTGGGGTTC CCGATCGCTT TTCTGGTTCT GGATCGGGGA CAGACTTCAC TCTGAAGATT
241 AGCCGCGTTG AAGCCGAAGA TCTGGGCGTG TACTTCTGCT TTCAAGGGAC GCATGTGCCG
301 TATACCTTTG GCGGTGGGAC TCGCCTGGAA ATCAAAGGAG GAGGCGGCAG TGGAGGTGGC
361 GGTAGTGGTG GCGGTGGCTC ACAGATTCAG CTGCAGCAAT CTGGTCCAGA GCTTGTTCGT
421 CCTGGCGCAT CAGTGAAAAT CTCGTGCAAA GCATCCGGTT ACACCTTTAC GGACTATTAC
481 ATCCATTGGG TGAAACAACG TCCTGGTGAA GGTTTGGAAT GGATTGGTTG GATTTATCCG
541 GGCAGCGGCA ACACCAAGTA TAACGAGAAG TTCAAAGGCA AAGCCACTCT CACCGTGGAT
601 ACATCGTCCA GCACCGCTTA CATGCAGCTG AGTTCTCTGA CCTCTGAAGA TTCCGCGGTC
661 TATTTCTGTG CTCGTGGTGG CAAATTTGCG ATGGACTATT GGGGCCAAGG CACCAGCGTA
721 ACCGTGTCAT CCTAG
```

with the immunoglobulin light chain variable region shown in bold, the glysine-serine linker region shown in italics, and the immunoglobulin heavy chain variable region shown in underline.

The partial amino acid sequence of scFV735 (GenBank Accession No. BAN21718.1; SEQ ID NO: 18) is as follows:

```
    1 DVVMTQTPLS LPVSLGDQAS ISCRSSQSLV HSNGNTYLYW YLQKPGQSPK PLIYRVSNRF
  61 SGVPDRFSGS GSGTDFTLKI SRVEAEDLGV YFCFQGTHVP YTFGGGTRLE IKGGGGSGGG
121 GSGGGGSQIQ LQQSGPELVR PGASVKISCK ASGYTFTDYY IHWVKQRPGE GLEWIGWIYP
181 GSGNTKYNEK FKGKATLTVD TSSSTAYMQL SSLTSEDSAV YFCARGGKFA MDYWGQGTSV
241 TVSS
```

### Example 5—Discussion

Here, it was sought to expand the knowledge base surrounding cell surface polySia and affirm its potential as a target for antibody-based cancer therapy. PolySia is a rare posttranslational modification that is found on a select group of identified carrier proteins including NCAM, SynCAM-1, Neuropilin-2, and the voltage sensitive sodium channel a subunit (Colley et al., “Polysialic Acid: Biosynthesis, Novel Functions and Applications,” Critical Reviews in Biochemistry and Molecular Biology 49:498-532 (2014), which is hereby incorporated by reference in its entirety). To create a more therapeutically relevant polySia-directed antibody, chimerized human mAb, ch735 was engineered, that was based on mouse-derived mo735, and determined that it recognized polySia with low nanomolar affinity and exquisite selectivity, binding α2,8-linked polySia structures with a DP of three sugar units or greater. It should be pointed out that previous experiments using SPR and ITC showed that mo735 prefers polySia chains of at least 8-11 sialic acid residues with increasing affinity as length increases (Hayrinen et al., “High Affinity Binding of Long-Chain Polysialic Acid to Antibody, and Modulation by Divalent Cations and Polyamines,” Mol. Immunol. 39:399-411 (2002), which is hereby incorporated by reference in its entirety), in line with applicants' glycoprotein microarray results with polySia-NCAM that has a DP of ˜50 (Hayrinen et al., “High Affinity Binding of Long-Chain Polysialic Acid to Antibody, and Modulation by Divalent Cations and Polyamines,” Mol. Immunol. 39:399-411 (2002), which is hereby incorporated by reference in its entirety). The binding to much shorter polySia chains that was observed with the glycan microarray could be due to differences in sensitivity and/or in how the immobilized glycans were presented to the antibody for binding (i.e., clustered). It is worth noting that a single-chain Fv (scFv) antibody derived from mo735 was observed to bind shorter a2-8-linked sialic acids (DP ˜3) (Nagae et al., “Crystal Structure of Anti-Polysialic Acid Antibody Single Chain Fv Fragment Complexed with Octasialic Acid: Insight into the Binding Preference for Polysialic Acid,” J. Biol. Chem. 288:33784-96 (2013), which is hereby incorporated by reference in its entirety), which likely explains why ch735 and mo735 both bind to the shorter polySia structures on the glycan array. It was proposed that mo735 recognizes sialic acid trisaccharide units in a paired manner and that this lends itself to higher affinities for longer chains.

Using this chimeric human mAb, high levels of polySia expression on several different cancer cell lines was confirmed, in agreement with earlier findings that this aberrant glycan is abundantly expressed on human cancers. Importantly, polySia-positive tumor cells were observed to rapidly internalize ch735 in endosomal and lysosomal compartments. In this regard, it should be pointed out that NCAM, one of the major polySia carrier proteins, is well known to undergo internalization via the clathrin-dependent endocytic pathway in astrocytes, cortical neurons, and rat neuroblastoma cells (Diestel et al., “NCAM is Ubiquitylated, Endocytosed and Recycled in Neurons,” J. Cell Sci. 120:4035-49 (2007) and Minana et al., “Neural Cell Adhesion Molecule is Endocytosed Via a Clathrin-Dependent Pathway,” Eur. J. Neurosci. 13:749-56 (2001), which are hereby incorporated by reference in their entirety). However, studies of Wilms tumor revealed that while NCAM was similarly present in intracellular compartments from the nuclear membrane to the plasma membrane, polySia was only detectable at the cell surface (Zuber et al., “The Relationship of Polysialic Acid and the Neural Cell Adhesion Molecule N-CAM in Wilms Tumor and their Subcellular Distributions,” Eur. J. Cell Biol. 51:313-21 (1990), which is hereby incorporated by reference in its entirety). Likewise, polySia was found exclusively on the surface of SW2 cells (Martersteck et al., “Unique Alpha 2, 8-Polysialylated Glycoproteins in Breast Cancer and Leukemia Cells,” Glycobiology 6:289-301 (1996), which is hereby incorporated by reference in its entirety). A more recent investigation of polySia turnover in human rhabdomyosarcoma cells reported that small numbers of polySia-NCAM molecules were recurrently found co-localizing with Rab5 (early endocytic marker), but only upon activation by the extracellular matrix (ECM) (Monzo et al., “Insulin and IGF1 Modulate Turnover of Polysialylated Neural Cell Adhesion Molecule (PSA-NCAM) in a Process Involving Specific Extracellular Matrix Components,” J. Neurochem 126:758-70 (2013), which is hereby incorporated by reference in its entirety). The absence of detectable constitutive internalization of polySia in these studies led to the belief in this case that the rapid internalization following ch735 binding observed here is an instance of antibody-induced receptor internalization (Tarcic et al., “Antibody-Mediated Receptor Endocytosis: Harnessing the Cellular Machinery to Combat Cancer,” In: Y. Y, G. T, Editors. Vesicle Trafficking in Cancer. New York, N.Y.: Springer; 2013, which is hereby incorporated by reference in its entirety). Interestingly, polySia-binding Escherichia coli bacteriophages were similarly reported to induce endocytosis of polySia in human neuroblastoma cells, whereas polySia remained at the cell surface if no phage was added (Lehti et al., “Internalization of a Polysialic Acid-Binding Escherichia Coli Bacteriophage into Eukaryotic Neuroblastoma Cells,” Nat. Commun. 8:1915 (2017), which is hereby incorporated by reference in its entirety).

The ability of experimental and therapeutic antibodies to induce endocytosis of their antigens is a commonly observed phenomenon that has been leveraged as a strategy to internalize oncogenic (or survival-mediating) antigens for eliciting anti-tumor effects or to deliver cytotoxic payloads directly into cancer cells (Tarcic et al., “Antibody-Mediated Receptor Endocytosis: Harnessing the Cellular Machinery to Combat Cancer,” In: Y. Y, G. T, Editors. Vesicle Trafficking in Cancer. New York, N.Y.: Springer; 2013, which is hereby incorporated by reference in its entirety). In the case of the latter, polySia possesses a number of attributes that make it an ideal target for an ADC including: (1) it is abundantly and selectively expressed on cancer cells as discussed above; (2) it is not detected in extracellular supernatants (Monzo et al., “Insulin and IGF1 Modulate Turnover of Polysialylated Neural Cell Adhesion Molecule (PSA-NCAM) in a Process Involving Specific Extracellular Matrix Components,” J. Neurochem 126:758-70 (2013), which is hereby incorporated by reference in its entirety), and the NCAM ectodomains that are shed from the cell surface are devoid of polySia (Hinkle et al., “Metalloprotease-Induced Ectodomain Shedding of Neural Cell Adhesion Molecule (NCAM),” J. Neurobiol. 66:1378-95 (2006), which is hereby incorporated by reference in its entirety); (3) it possesses an appropriate rate of endocytosis, comparable to that measured previously for trastuzumab (Li et al., “A Biparatopic HER2-Targeting Antibody-Drug Conjugate Induces Tumor Regression in Primary Models Refractory to or Ineligible for HER2-Targeted Therapy,” Cancer Cell 29:117-29 (2016), which is hereby incorporated by reference in its entirety); and (4) it is trafficked to the endolysosomal degradation pathway and retained in a maturing endosome (rather than being recycled back to the plasma membrane) (Monzo et al., “Insulin and IGF1 Modulate Turnover of Polysialylated Neural Cell Adhesion Molecule (PSA-NCAM) in a Process Involving Specific Extracellular Matrix Components,” J. Neurochem 126:758-70 (2013), which is hereby incorporated by reference in its entirety) until finally being delivered to the lysosome, an appropriate intracellular trafficking route when using a non-cleavable linker (Ritchie et al., “Implications of Receptor-Mediated Endocytosis and Intracellular Trafficking Dynamics in the Development of Antibody Drug Conjugates,” mAbs 5:13-21 (2013), which is hereby incorporated by reference in its entirety). To harness these traits, an ADC was synthesized in which Tz-modified ch735 was bioorthogonally conjugated to the TCO-maleimide-DM1 drug linker. The resulting ch735-Py-DM1 conjugate exhibited potent polySia-specific cytotoxicity in vitro, rivaling the potency of a similarly synthesized T-Py-DM1 conjugate. To the inventors' knowledge, this is the first ADC that targets an N-linked glycan epitope on the surface of cancer cells and one of few to leverage the Tz/TCO bioorthogonal click chemistry described here. The relative ease of component synthesis and fast reaction kinetics of this two-step method allows for rapid generation of ADCs against new targets. Additionally, the aromatic stability of the pyridazine product formed could aid in stability (Selvaraj et al., “Trans-Cyclooctene—a Stable, Voracious Dienophile for Bioorthogonal Labeling,” Curr. Opin. Chem. Biol. 17:753-60 (2013), which is hereby incorporated by reference in its entirety). This is even more significant when one considers that unconjugated (‘naked’) mo735 exhibited only limited complement-dependent cytotoxicity (CDC) against cultured neurons (Pon et al., “Polysialic Acid Bioengineering of Neuronal Cells by N-Acyl Sialic Acid Precursor Treatment,” Glycobiology 17:249-60 (2007), which is hereby incorporated by reference in its entirety), while mAb 5A5, a polySia-specific IgM, exhibited no measurable CDC against several different SCLC cell lines (Livingston et al., “Selection of GM2, Fucosyl GM1, Globo H and Polysialic Acid as Targets on Small Cell Lung Cancers for Antibody Mediated Immunotherapy,” Cancer Immuno.l Immunother. 54:1018-25 (2005), which is hereby incorporated by reference in its entirety).

It is worth mentioning that the IC₅₀ value measured for ch735-Py-DM1 compared favorably to a number of previously reported ADCs against protein antigens including HER2 and NCAM, as well as a small handful of ADCs that target cell surface O-glycans including STn, Tn, and T, the blood group-related Lewis Y antigen (Table 2). This latter group, together with the ch735-Py-DM1 conjugate, represents a new class of glycan-directed ADCs that hold promise for anti-tumor therapy. It is anticipated that the availability of antibodies such as ch735 that recognize aberrantly expressed tumor glycans should aid the development of novel glycan-directed synthetic immunotherapies for specifically focusing immune or immune-like responses on the tumor glycocalyx. While the focus here is on engineering a glycan-specific ADC, it is envisioned that molecular reformatting of antibodies or antibody domains could be used to create next-generation glycan-directed immunotherapies including BsAbs or CAR-T cells (Steentoft et al., “Glycan-Directed CAR-T Cells,” Glycobiology 28:656-69 (2018) and Xu et al., “Retargeting T Cells to GD2 Pentasaccharide on Human Tumors using Bispecific Humanized Antibody,” Cancer Immunol. Res. 3:266-77 (2015), which are hereby incorporated by reference in their entirety).

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. An immunoconjugate therapeutic comprising:
a polysialic acid targeting portion and
an anti-cancer therapeutic coupled to the polysialic acid targeting portion.

2. The immunoconjugate therapeutic of claim 1, wherein the polysialic acid targeting portion is a mammalian polysialic acid targeting portion.

3. The immunoconjugate therapeutic of claim 2, wherein the polysialic acid targeting portion is a human polysialic acid targeting portion.

4. The immunoconjugate therapeutic of claim 1, wherein the polysialic acid targeting portion is selected from the group consisting of a full-length immunoglobulin and a binding portion thereof which binds to polysialic acid.

5. The immunoconjugate therapeutic of claim 4, wherein the full-length immunoglobulin is selected from the group consisting of immunoglobulin G1 (IgG1), immunoglobulin G2 (IgG2), immunoglobulin G3 (IgG3), immunoglobulin G4 (IgG4), immunoglobulin M (IgM), immunoglobulin E (IgE), immunoglobulin D (IgD), and immunoglobulin A (IgA), and wherein the full-length immunoglobulin binds to polysialic acid.

6. The immunoconjugate therapeutic of claim 4, wherein the polysialic acid targeting portion is selected from the group consisting of a single chain variable fragment (scFv), a single chain antibody fragment (scab), a single domain antibody (dAb), a fragment antigen binding (Fab) fragment, a Fab′ fragment, F(ab′)₂ fragment, a single-chain Fv fused to Fc domain (scFv-Fc), a single domain antibody fused to Fc domain (dAb-Fc), a free light chain (free LC), a half antibody, and derivatives thereof, and wherein the targeting portion binds to polysialic acid.

7. The immunoconjugate therapeutic of claim 1, wherein the polysialic acid targeting portion is a monoclonal antibody or a polyclonal antibody.

8. The immunoconjugate therapeutic of claim 1, wherein the polysialic acid targeting portion is a mouse, human, chimeric, or humanized monoclonal antibody.

9. The immunoconjugate therapeutic of claim 8, wherein the polysialic acid targeting portion is monoclonal antibody mo735.

10. The immunoconjugate therapeutic of claim 8, wherein the polysialic acid targeting portion is a derivative of monoclonal antibody mo735 which binds to polysialic acid.

11. The immunoconjugate therapeutic of claim 10, wherein the polysialic acid targeting portion is monoclonal antibody ch735.

12. The immunoconjugate therapeutic of claim 1, wherein the polysialic acid targeting portion comprises a light chain variable region and a heavy chain variable region, wherein said light chain variable region has an amino acid sequence comprising SEQ ID NO: 1 and said heavy chain variable region has an amino acid sequence comprising SEQ ID NO: 2.

13. The immunoconjugate therapeutic of claim 1, wherein the polysialic acid targeting portion comprises a light chain variable region and a heavy chain variable region, wherein said light chain variable region is encoded by a nucleic acid sequence comprising SEQ ID NO: 3 and said heavy chain variable region is encoded by a nucleic acid sequence comprising SEQ ID NO: 4.

14. The immunoconjugate therapeutic of claim 1, wherein the anti-cancer therapeutic is selected from the group consisting of microtubule disrupting agents, DNA modifying agents, RNA modifying agents, DNA damaging agents, and RNA damaging agents.

15. The immunoconjugate therapeutic of claim 14, wherein the anti-cancer therapeutic is selected from the group consisting of maytansinoids, auristatins, tubulysins, duocarymycins, calicheamicins, pyrrolobenzodiazepines, radionuclides, amatoxins, camptothecins, doxorubicin, 5-fluorouracil, and methotrexate.

16. The immunoconjugate therapeutic of claim 15, wherein the anti-cancer therapeutic is a maytansinoid.

17. The immunoconjugate therapeutic of claim 16, wherein the maytansinoid is selected from the group consisting of emtansine (DM1) and ravtansine (DM4).

18. The immunoconjugate therapeutic of claim 1, wherein the therapeutic is monoclonal antibody ch735 coupled to maytansinoid DM1.

19. The immunoconjugate therapeutic of claim 1, wherein the polysialic acid targeting portion is coupled to the anti-cancer therapeutic through a linker element.

20. The immunoconjugate therapeutic of claim 19 wherein the linker element is selected from the group consisting of cleavable and non-cleavable linkers.

21. The immunoconjugate therapeutic of claim 20, wherein the linker element is capable of being synthesized via a bioorthogonal conjugation reaction.

22. The immunoconjugate therapeutic of claim 21, wherein linker element comprises 1,4-dihydropyridazine (Py) conjugation moiety element capable of synthesis via a biorthogonal conjugation reaction.

23. A method of treating subjects with cancer, said method comprising:
selecting a subject with cancer characterized by polysialic acid (polySia)-positive tumor cells and administering the immunoconjugate therapeutic of claim 1 to the selected subject.

24.-34. (canceled)

35. A method of targeted intracellular delivery of an anti-cancer therapeutic to a target cell population, said method comprising:
selecting a population of target cells, wherein the population of target cells is positive for polysialic acid (polySia) and
administering the immunoconjugate therapeutic of claim 1 to the selected target cell population.

36.-49. (canceled)

50. A pharmaceutical composition comprising:
the immunoconjugate therapeutic of claim 1; and
a carrier.


SEQUENCE LISTING (18 sequences)

SEQ ID NO: 1 - protein, 109 aa, Artificial
  Synthetic Sequence
MTQTPLSLPVSLGDQASISCRSSQSLVHSNGNTYLYWYLQKPGQSPKPLIYRVSNRFSGVPDRFSGSGSGTDFTLKISRVEAEDLGVYFCFQGTHVPYTFGGGTRLEIK

SEQ ID NO: 2 - protein, 117 aa, Artificial
  Synthetic Sequence
QIQLQQSGPELVRPGASVKISCKASGYTFTDYYIHWVKQRPGEGLEWIGWIYPGSGNTKYNEKFKGKATLTVDTSSSTAYMQLSSLTSEDSAVYFCARGGKFAMDYWGQGTSVTVSS

SEQ ID NO: 3 - DNA, 336 bp, Artificial
  Synthetic Sequence
gatgtagtcatgacgcagacgccacttagcttaccggtcagtttaggggatcaggcgagcattagctgtcgctcctcacagagcttggttcacagcaatgggaacacgtacctgtactggtatctgcagaaaccgggccaatcgccgaaaccgctcatctatcgggtatcgaatcgctttagtggggttcccgatcgcttttctggttctggatcggggacagacttcactctgaagattagccgcgttgaagccgaagatctgggcgtgtacttctgctttcaagggacgcatgtgccgtatacctttggcggtgggactcgcctggaaatcaaa

SEQ ID NO: 4 - DNA, 351 bp, Artificial
  Synthetic Sequence
cagattcagctgcagcaatctggtccagagcttgttcgtcctggcgcatcagtgaaaatctcgtgcaaagcatccggttacacctttacggactattacatccattgggtgaaacaacgtcctggtgaaggtttggaatggattggttggatttatccgggcagcggcaacaccaagtataacgagaagttcaaaggcaaagccactctcaccgtggatacatcgtccagcaccgcttacatgcagctgagttctctgacctctgaagattccgcggtctatttctgtgctcgtggtggcaaatttgcgatggactattggggccaaggcaccagcgtaaccgtgtcatcc

SEQ ID NO: 5 - DNA, 20 bp, Artificial
  Synthetic Sequence
atgcagtgcgcacgttgacg

SEQ ID NO: 6 - DNA, 20 bp, Artificial
  Synthetic Sequence
acccgatgagttgcgtctcc

SEQ ID NO: 7 - DNA, 40 bp, Artificial
  Synthetic Sequence
cgtacggtggcggcgccatctgtcttcatcttcccgccat

SEQ ID NO: 8 - DNA, 40 bp, Artificial
  Synthetic Sequence
ggagtgcgcgcctgtggcggccgccaccaagaagaggatc

SEQ ID NO: 9 - DNA, 40 bp, Artificial
  Synthetic Sequence
gctagcacacagagcccatccgtcttccccttgacccgct

SEQ ID NO: 10 - DNA, 40 bp, Artificial
  Synthetic Sequence
accgcggctagctggaacccagagcagcagaaacccaatg

SEQ ID NO: 11 - DNA, 40 bp, Artificial
  Synthetic Sequence
gggttccagctagccgcggtgatgtagtcatgacgcagac

SEQ ID NO: 12 - DNA, 41 bp, Artificial
  Synthetic Sequence
gggactcgcctggaaatcaaacgtacggtggcggcgccatc

SEQ ID NO: 13 - DNA, 40 bp, Artificial
  Synthetic Sequence
ccgccacaggcgcgcactcccagattcagctgcagcaatc

SEQ ID NO: 14 - DNA, 40 bp, Artificial
  Synthetic Sequence
ccagcgtaaccgtgtcatccgctagcaccaagggcccatc

SEQ ID NO: 15 - DNA, 8589 bp, Artificial
  Synthetic Sequence
cctgcagggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccactagtggagccgagagtaattcatacaaaaggagggatcgccttcgcaaggggagagcccagggaccgtccctaaattctcacagacccaaatccctgtagccgccccacgacagcgcgaggagcatgcgctcagggctgagcgcggggagagcagagcacacaagctcatagaccctggtcgtgggggggaggaccggggagctggcgcggggcaaactgggaaagcggtgtcgtgtgctggctccgccctcttcccgagggtgggggagaacggtatataagtgcggcagtcgccttggacgttctttttcgcaacgggtttgccgtcagaacgcaggtgaggggcgggtgtggcttccgcgggccgccgagctggaggtcctgctccgagcgggccgggccccgctgtcgtcggcggggattagctgcgagcattcccgcttcgagttgcgggcggcgcgggaggcagagtgcgaggcctagcggcaaccccgtagcctcgcctcgtgtccggcttgaggcctagcgtggtgtccgcgccgccgccgcgtgctactccggccgcactctggtcttttttttttttgttgttgttgccctgctgccttcgattgccgttcagcaataggggctaacaaagggagggtgcggggcttgctcgcccggagcccggagaggtcatggttggggaggaatggagggacaggagtggcggctggggcccgcccgccttcggagcacatgtccgacgccacctggatggggcgaggcctggggtttttcccgaagcaaccaggctggggttagcgtgccgaggccatgtggccccagcacccggcacgatctggcttggcggcgccgcgttgccctgcctccctaactagggtgaggccatcccgtccggcaccagttgcgtgcgtggaaagatggccgctcccgggccctgttgcaaggagctcaaaatggaggacgcggcagcccggtggagcgggcgggtgagtcacccacacaaaggaagagggcctggtccctcaccggctgctgcttcctgtgaccccgtggtcctatcggccgcaatagtcacctcgggcttttgagcacggctagtcgcggcggggggaggggatgtaatggcgttggagtttgttcacatttggtgggtggagactagtcaggccagcctggcgctggaagtcatttttggaatttgtccccttgagttttgagcggagctaattctcgggcttcttagcggttcaaaggtatcttttaaacccttttttaggtgttgtgaaaaccaccgctaattcaaagcaaccggtatggactggacctggaggatcctcttcttggtggcggccgccacaggcgcgcactcccagattcagctgcagcaatctggtccagagcttgttcgtcctggcgcatcagtgaaaatctcgtgcaaagcatccggttacacctttacggactattacatccattgggtgaaacaacgtcctggtgaaggtttggaatggattggttggatttatccgggcagcggcaacaccaagtataacgagaagttcaaaggcaaagccactctcaccgtggatacatcgtccagcaccgcttacatgcagctgagttctctgacctctgaagattccgcggtctatttctgtgctcgtggtggcaaatttgcgatggactattggggccaaggcaccagcgtaaccgtgtcatccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgatgtacagctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaaatgttaattaactagccatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttcttaattaacctgcaggcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgatgatgcggttttggcagtacatcaatgggcgtggatagcggtttgactcacggggatttccaagtctccaccccattgacgtcaatgggagtttgttttgactagtggagccgagagtaattcatacaaaaggagggatcgccttcgcaaggggagagcccagggaccgtccctaaattctcacagacccaaatccctgtagccgccccacgacagcgcgaggagcatgcgcccagggctgagcgcgggtagatcagagcacacaagctcacagtccccggcggtggggggaggggcgcgctgagcgggggccagggagctggcgcggggcaaactgggaaagtggtgtcgtgtgctggctccgccctcttcccgagggtgggggagaacggtatataagtgcggtagtcgccttggacgttctttttcgcaacgggtttgccgtcagaacgcaggtgagtggcgggtgtggcttccgcgggccccggagctggagccctgctctgagcgggccgggctgatatgcgagtgtcgtccgcagggtttagctgtgagcattcccacttcgagtggcgggcggtgcgggggtgagagtgcgaggcctagcggcaaccccgtagcctcgcctcgtgtccggcttgaggcctagcgtggtgtccgccgccgcgtgccactccggccgcactatgcgttttttgtccttgctgccctcgattgccttccagcagcatgggctaacaaagggagggtgtggggctcactcttaaggagcccatgaagcttacgttggataggaatggaagggcaggaggggcgactggggcccgcccgccttcggagcacatgtccgacgccacctggatggggcgaggcctgtggctttccgaagcaatcgggcgtgagtttagcctacctgggccatgtggccctagcactgggcacggtctggcctggcggtgccgcgttcccttgcctcccaacaagggtgaggccgtcccgcccggcaccagttgcttgcgcggaaagatggccgctcccggggccctgttgcaaggagctcaaaatggaggacgcggcagcccggtggagcgggcgggtgagtcacccacacaaaggaagagggccttgcccctcgccggccgctgcttcctgtgaccccgtggtctatcggccgcatagtcacctcgggcttctcttgagcaccgctcgtcgcggcggggggaggggatctaatggcgttggagtttgttcacatttggtgggtggagactagtcaggccagcctggcgctggaagtcattcttggaatttgcccctttgagtttggagcgaggctaattctcaagcctcttagcggttcaaaggtattttctaaacccgtttccaggtgttgtgaaagccaccgctaattcaaagcaatccggaatgttgccatcacaactcattgggtttctgctgctctgggttccagctagccgcggtgatgtagtcatgacgcagacgccacttagcttaccggtcagtttaggggatcaggcgagcattagctgtcgctcctcacagagcttggttcacagcaatgggaacacgtacctgtactggtatctgcagaaaccgggccaatcgccgaaaccgctcatctatcgggtatcgaatcgctttagtggggttcccgatcgcttttctggttctggatcggggacagacttcactctgaagattagccgcgttgaagccgaagatctgggcgtgtacttctgctttcaagggacgcatgtgccgtatacctttggcggtgggactcgcctggaaatcaaacgtacggtggcggcgccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagggatcccgtacgcctaggagcaggtttccccaatgacacaaaacgtgcaacttgaaactccgcctggtctttccaggtctagaggggtaacactttgtactgcgtttggctccacgctcgatccactggcgagtgttagtaacagcactgttgcttcgtagcggagcatgacggccgtgggaactcctccttggtaacaaggacccacggggccaaaagccacgcccacacgggcccgtcatgtgtgcaaccccagcacggcgactttactgcgaaacccactttaaagtgacattgaaactggtacccacacactggtgacaggctaaggatgcccttcaggtaccccgaggtaacacgcgacactcgggatctgagaaggggactggggcttctataaaagcgctcggtttaaaaagcttctatgcctgaataggtgaccggaggtcggcacctttcctttgcaattactgaccctatgaatacactgactgtttgacaattaatcatcggcatagtatatcggcatagtataatacgactcactataggagggccaccatgaagaaacctgaactgacagcaacttctgttgagaagtttctcattgaaaaatttgattctgtttctgatctcatgcagctgtctgaaggtgaagaaagcagagccttttcttttgatgttggaggaagaggttatgttctgagggtcaattcttgtgctgatggtttttacaaagacagatatgtttacagacactttgcctctgctgctctgccaattccagaagttctggacattggagaattttctgaatctctcacctactgcatcagcagaagagcacaaggagtcactctccaggatctccctgaaactgagctgccagctgttctgcaacctgttgctgaagcaatggatgccattgcagcagctgatctgagccaaacctctggatttggtccttttggtccccaaggcattggtcagtacaccacttggagggatttcatttgtgccattgctgatcctcatgtctatcactggcagactgtgatggatgacacagtttctgcttctgttgctcaggcactggatgaactcatgctgtgggcagaagattgtcctgaagtcagacacctggtccatgctgattttggaagcaacaatgttctgacagacaatggcagaatcactgcagtcattgactggtctgaagccatgtttggagattctcaatatgaggttgccaacatttttttttggagaccttggctggcttgcatggaacaacaaacaagatattttgaaagaagacacccagaactggctggttcccccagactgagagcctacatgctcagaattggcctggaccaactgtatcaatctctggttgatggaaactttgatgatgctgcttgggcacaaggaagatgtgatgccattgtgaggtctggtgctggaactgttggaagaactcaaattgcaagaaggtctgctgctgtttggactgatggatgtgttgaagttctggctgactctggaaacaggagaccctccacaagacccagagccaaggaatgaatattagctagattatccctaatacctgccaccccactcttaatcagtggtggaagaacggtctcagaactgtttgtttcaattggccatttaagtttagtagtaaaagactggttaatgataacaatgcatcgtaaaaccttcagaaggaaaggagaatgttttgtggaccactttggttttcttttttgcgtgtggcagttttaagttattagtttttaaaatcagtactttttaatggaaacaacttgaccaaaaatttgtcacagaattttgagacccattaaaaaagttaaatgagaaacctgtgtgttcctttggtcaacaccgagacatttaggtgaaagacatctaattctggttttacgaatctggaaacttcttgaaaatgtaattcttgagttaacacttctgggtggagaatagggttgttttccccccacataattggaaggggaaggaatatcatttaaagctatgggagggttgctttgattacaacactggagagaaatgcagcatgttgctgattgcctgtcactaaaacaggccaaaaactgagtccttgggttgcatagaaagctg

SEQ ID NO: 16 - DNA, 8583 bp, Artificial
  Synthetic Sequence
cctgcagggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccactagtggagccgagagtaattcatacaaaaggagggatcgccttcgcaaggggagagcccagggaccgtccctaaattctcacagacccaaatccctgtagccgccccacgacagcgcgaggagcatgcgctcagggctgagcgcggggagagcagagcacacaagctcatagaccctggtcgtgggggggaggaccggggagctggcgcggggcaaactgggaaagcggtgtcgtgtgctggctccgccctcttcccgagggtgggggagaacggtatataagtgcggcagtcgccttggacgttctttttcgcaacgggtttgccgtcagaacgcaggtgaggggcgggtgtggcttccgcgggccgccgagctggaggtcctgctccgagcgggccgggccccgctgtcgtcggcggggattagctgcgagcattcccgcttcgagttgcgggcggcgcgggaggcagagtgcgaggcctagcggcaaccccgtagcctcgcctcgtgtccggcttgaggcctagcgtggtgtccgcgccgccgccgcgtgctactccggccgcactctggtcttttttttttttgttgttgttgccctgctgccttcgattgccgttcagcaataggggctaacaaagggagggtgcggggcttgctcgcccggagcccggagaggtcatggttggggaggaatggagggacaggagtggcggctggggcccgcccgccttcggagcacatgtccgacgccacctggatggggcgaggcctggggtttttcccgaagcaaccaggctggggttagcgtgccgaggccatgtggccccagcacccggcacgatctggcttggcggcgccgcgttgccctgcctccctaactagggtgaggccatcccgtccggcaccagttgcgtgcgtggaaagatggccgctcccgggccctgttgcaaggagctcaaaatggaggacgcggcagcccggtggagcgggcgggtgagtcacccacacaaaggaagagggcctggtccctcaccggctgctgcttcctgtgaccccgtggtcctatcggccgcaatagtcacctcgggcttttgagcacggctagtcgcggcggggggaggggatgtaatggcgttggagtttgttcacatttggtgggtggagactagtcaggccagcctggcgctggaagtcatttttggaatttgtccccttgagttttgagcggagctaattctcgggcttcttagcggttcaaaggtatcttttaaacccttttttaggtgttgtgaaaaccaccgctaattcaaagcaaccggtatggactggacctggaggatcctcttcttggtggcggccgccacaggcgcgcactccgaggtgcagctggtggagtctggtggtggtcttgttcaacctggtggttctcttcgtctttcttgtgctgcttctggttttaatattaaagatacttatattcattgggttcgtcaagctcctggtaaaggtcttgaatgggttgctcgtatttatcctactaatggttatactcgttatgctgattctgttaaaggtcgttttactatttctgctgatacttctaaaaatactgcttatcttcaaatgaactctcttcgtgctgaagatactgctgtttattattgttctcgttggggtggtgatggtttttatgctatggattattggggtcaaggtactcttgtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgatgtacagctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaaatgttaattaactagccatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttcttaattaacctgcaggcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgatgatgcggttttggcagtacatcaatgggcgtggatagcggtttgactcacggggatttccaagtctccaccccattgacgtcaatgggagtttgttttgactagtggagccgagagtaattcatacaaaaggagggatcgccttcgcaaggggagagcccagggaccgtccctaaattctcacagacccaaatccctgtagccgccccacgacagcgcgaggagcatgcgcccagggctgagcgcgggtagatcagagcacacaagctcacagtccccggcggtggggggaggggcgcgctgagcgggggccagggagctggcgcggggcaaactgggaaagtggtgtcgtgtgctggctccgccctcttcccgagggtgggggagaacggtatataagtgcggtagtcgccttggacgttctttttcgcaacgggtttgccgtcagaacgcaggtgagtggcgggtgtggcttccgcgggccccggagctggagccctgctctgagcgggccgggctgatatgcgagtgtcgtccgcagggtttagctgtgagcattcccacttcgagtggcgggcggtgcgggggtgagagtgcgaggcctagcggcaaccccgtagcctcgcctcgtgtccggcttgaggcctagcgtggtgtccgccgccgcgtgccactccggccgcactatgcgttttttgtccttgctgccctcgattgccttccagcagcatgggctaacaaagggagggtgtggggctcactcttaaggagcccatgaagcttacgttggataggaatggaagggcaggaggggcgactggggcccgcccgccttcggagcacatgtccgacgccacctggatggggcgaggcctgtggctttccgaagcaatcgggcgtgagtttagcctacctgggccatgtggccctagcactgggcacggtctggcctggcggtgccgcgttcccttgcctcccaacaagggtgaggccgtcccgcccggcaccagttgcttgcgcggaaagatggccgctcccggggccctgttgcaaggagctcaaaatggaggacgcggcagcccggtggagcgggcgggtgagtcacccacacaaaggaagagggccttgcccctcgccggccgctgcttcctgtgaccccgtggtctatcggccgcatagtcacctcgggcttctcttgagcaccgctcgtcgcggcggggggaggggatctaatggcgttggagtttgttcacatttggtgggtggagactagtcaggccagcctggcgctggaagtcattcttggaatttgcccctttgagtttggagcgaggctaattctcaagcctcttagcggttcaaaggtattttctaaacccgtttccaggtgttgtgaaagccaccgctaattcaaagcaatccggaatgttgccatcacaactcattgggtttctgctgctctgggttccagctagccgcggtgacatccagatgacccagtctccttcttctctttctgcttctgttggtgatcgtgttactattacttgtcgtgcttctcaagatgttaatactgctgttgcttggtatcaacaaaaacctggtaaagctcctaaacttcttatttattctgcttcttttctttattctggtgttccttctcgtttttctggttctcgttctggtactgattttactcttactatttcttctcttcaacctgaagattttgctacttattattgtcaacaacattatactactcctcctacttttggtcaaggtaccaagctggagatcaaacgtacggtggcggcgccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagggatcccgtacgcctaggagcaggtttccccaatgacacaaaacgtgcaacttgaaactccgcctggtctttccaggtctagaggggtaacactttgtactgcgtttggctccacgctcgatccactggcgagtgttagtaacagcactgttgcttcgtagcggagcatgacggccgtgggaactcctccttggtaacaaggacccacggggccaaaagccacgcccacacgggcccgtcatgtgtgcaaccccagcacggcgactttactgcgaaacccactttaaagtgacattgaaactggtacccacacactggtgacaggctaaggatgcccttcaggtaccccgaggtaacacgcgacactcgggatctgagaaggggactggggcttctataaaagcgctcggtttaaaaagcttctatgcctgaataggtgaccggaggtcggcacctttcctttgcaattactgaccctatgaatacactgactgtttgacaattaatcatcggcatagtatatcggcatagtataatacgactcactataggagggccaccatgaagaaacctgaactgacagcaacttctgttgagaagtttctcattgaaaaatttgattctgtttctgatctcatgcagctgtctgaaggtgaagaaagcagagccttttcttttgatgttggaggaagaggttatgttctgagggtcaattcttgtgctgatggtttttacaaagacagatatgtttacagacactttgcctctgctgctctgccaattccagaagttctggacattggagaattttctgaatctctcacctactgcatcagcagaagagcacaaggagtcactctccaggatctccctgaaactgagctgccagctgttctgcaacctgttgctgaagcaatggatgccattgcagcagctgatctgagccaaacctctggatttggtccttttggtccccaaggcattggtcagtacaccacttggagggatttcatttgtgccattgctgatcctcatgtctatcactggcagactgtgatggatgacacagtttctgcttctgttgctcaggcactggatgaactcatgctgtgggcagaagattgtcctgaagtcagacacctggtccatgctgattttggaagcaacaatgttctgacagacaatggcagaatcactgcagtcattgactggtctgaagccatgtttggagattctcaatatgaggttgccaacatttttttttggagaccttggctggcttgcatggaacaacaaacaagatattttgaaagaagacacccagaactggctggttcccccagactgagagcctacatgctcagaattggcctggaccaactgtatcaatctctggttgatggaaactttgatgatgctgcttgggcacaaggaagatgtgatgccattgtgaggtctggtgctggaactgttggaagaactcaaattgcaagaaggtctgctgctgtttggactgatggatgtgttgaagttctggctgactctggaaacaggagaccctccacaagacccagagccaaggaatgaatattagctagattatccctaatacctgccaccccactcttaatcagtggtggaagaacggtctcagaactgtttgtttcaattggccatttaagtttagtagtaaaagactggttaatgataacaatgcatcgtaaaaccttcagaaggaaaggagaatgttttgtggaccactttggttttcttttttgcgtgtggcagttttaagttattagtttttaaaatcagtactttttaatggaaacaacttgaccaaaaatttgtcacagaattttgagacccattaaaaaagttaaatgagaaacctgtgtgttcctttggtcaacaccgagacatttaggtgaaagacatctaattctggttttacgaatctggaaacttcttgaaaatgtaattcttgagttaacacttctgggtggagaatagggttgttttccccccacataattggaaggggaaggaatatcatttaaagctatgggagggttgctttgattacaacactggagagaaatgcagcatgttgctgattgcctgtcactaaaacaggccaaaaactgagtccttgggttgcatagaaagctg

SEQ ID NO: 17 - DNA, 735 bp, Artificial
  Synthetic Sequence
gatgtagtcatgacgcagacgccacttagcttaccggtcagtttaggggatcaggcgagcattagctgtcgctcctcacagagcttggttcacagcaatgggaacacgtacctgtactggtatctgcagaaaccgggccaatcgccgaaaccgctcatctatcgggtatcgaatcgctttagtggggttcccgatcgcttttctggttctggatcggggacagacttcactctgaagattagccgcgttgaagccgaagatctgggcgtgtacttctgctttcaagggacgcatgtgccgtatacctttggcggtgggactcgcctggaaatcaaaggaggaggcggcagtggaggtggcggtagtggtggcggtggctcacagattcagctgcagcaatctggtccagagcttgttcgtcctggcgcatcagtgaaaatctcgtgcaaagcatccggttacacctttacggactattacatccattgggtgaaacaacgtcctggtgaaggtttggaatggattggttggatttatccgggcagcggcaacaccaagtataacgagaagttcaaaggcaaagccactctcaccgtggatacatcgtccagcaccgcttacatgcagctgagttctctgacctctgaagattccgcggtctatttctgtgctcgtggtggcaaatttgcgatggactattggggccaaggcaccagcgtaaccgtgtcatcctag

SEQ ID NO: 18 - protein, 244 aa, Artificial
  Synthetic Sequence
DVVMTQTPLSLPVSLGDQASISCRSSQSLVHSNGNTYLYWYLQKPGQSPKPLIYRVSNRFSGVPDRFSGSGSGTDFTLKISRVEAEDLGVYFCFQGTHVPYTFGGGTRLEIKGGGGSGGGGSGGGGSQIQLQQSGPELVRPGASVKISCKASGYTFTDYYIHWVKQRPGEGLEWIGWIYPGSGNTKYNEKFKGKATLTVDTSSSTAYMQLSSLTSEDSAVYFCARGGKFAMDYWGQGTSVTVSS